(19) United States
(12) Patent Application Publication
(10) Pub. No.: US 2025/0032608 A1
(43) Pub. Date: Jan. 30, 2025

(54) TREATMENT OF ASTHMA WITH ANTI-TSL P ANTIBODY

(71) Applicants: AMGEN INC., Thousand Oaks, CA (US); MEDIMMUNE, LLC, Gaithersburg, MD (US)

(72) Inventors: Jane R. Parnes, Agoura Hills, CA (US); Janet Griffiths, Gaithersburg, MD (US)

(21) Appl. No.: 18/909,660

(22) Filed: Oct. 8, 2024

Related U.S. Application Data
(63) Continuation of application No. 17/093,387, filed on Nov. 9, 2020, now abandoned.
Application No. 17/093,387 is a division of application No. 15/951,602, filed on Apr. 12, 2018, now Pat. No. 10,828,365.
(60) Provisional application No. 62/553,477, filed on Sep. 1, 2017.
(60) Provisional application No. 62/553,575, filed on Sep. 1, 2017.
(60) Provisional application No. 62/484,864, filed on Apr. 12, 2017.

(51) Int. Cl. A61K 39/395; A61K 39/00; A61P 11/06; C07K 16/24

(52) U.S. Cl. CPC A61K 39/395; A61P 11/06; C07K 16/24; C07K 16/244; A61K 2039/505; A61K 2039/545; A61K 2039/55; C07K 2317/21; C07K 2317/565

(57) ABSTRACT

The present disclosure, relates, in general, to methods of treating asthma, including severe asthma and eosinophilic asthma, using an antibody specific for thymic stromal lymphopoietin (TSLP).

### CROSS REFERENCE TO RELATED APPLICATIONS

This application is a continuation of U.S. application Ser. No. 17/093,387 which is a Divisional of U.S. application Ser. No. 15/951,602 filed on Apr. 12, 2018, which claims the benefit of U.S. Provisional Application 62/553,477 filed on Sep. 1, 2017, U.S. Provisional Application 62/553,575 filed on Sep. 1, 2017, and U.S. Provisional Application 62/484,864 filed on Apr. 12, 2017, all of which are hereby incorporated by reference in their entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “51920D2_SeqListing.xml”, which was created on Oct. 8, 2024, and is 121,288 bytes in size. The subject matter of the Sequence Listing is incorporated herein in its entirety by reference.

### FIELD OF THE INVENTION

The present disclosure relates, in general, to methods of treating asthma, including severe asthma, eosinophilic asthma and non/low eosoniphilic asthma, using an antibody specific for thymic stromal lymphopoietin (TSLP).

### BACKGROUND

Asthma affects an estimated 315 million people worldwide.¹ Of these, approximately 10 to 15% have severe asthma² and as many as 60% have inadequately controlled disease.³ These patients are at risk for significantly impaired quality of life and recurrent severe exacerbations. Asthma therapies, including inhaled corticosteroids (ICS) combined with long-acting beta-2 agonists (LABA), may not provide adequate disease control, particularly in patients with severe disease.^{2,4,5} The heterogeneous response to asthma treatment, in part, may be related to differences in patterns of airway inflammation and resistance to corticosteroids.^{2,5,6} Alternative treatments that inhibit specific molecular targets, including immunoglobulin E (IgE), interleukin-4, interleukin-5, interleukin-13, and their respective receptors, have been shown to benefit some patients with asthma who are not fully controlled on optimal ICS/LABA therapy.⁷⁻¹⁸

Thymic stromal lymphopoietin (TSLP), an epithelial cell-derived cytokine produced in response to environmental and pro-inflammatory stimuli, leads to the activation of multiple inflammatory cells and downstream pathways.^{19,20} TSLP is increased in the airways of patients with asthma and correlates with Th2 cytokine and chemokine expression²¹ and disease severity.^{22,23} While TSLP is central to the regulation of Th2 immunity, it may also play a key role in other pathways of inflammation and therefore be relevant to multiple asthma phenotypes.

Tezepelumab is an human immunoglobulin G2 (IgG2) monoclonal antibody (mAb) that binds to TSLP, preventing its interaction with the TSLP receptor complex. A proof-of-concept study in patients with mild, atopic asthma, demonstrated that tezepelumab inhibited the early and late asthmatic responses and suppressed biomarkers of Th2 inflammation following inhaled allergen challenge.²⁴

The present disclosure describes a randomized, placebo-controlled, dose-ranging trial of tezepelumab in patients whose disease was inadequately controlled with medium to high doses of ICS/LABA.

### SUMMARY

The anti-TSLP antibody described herein addresses an unmet need in asthma patients in which other medications may not control moderate to severe asthma. For example, the antibody therapy may improve asthma in eosinophil (EOS)-low patients and may provide a more powerful exacerbation reduction in EOS-high patients.

The disclosure provides a method for treating asthma in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

Also contemplated is a method for treating asthma in a subject comprising administering a therapeutically effective amount of an anti-TSLP antibody or antibody variant in a dose of 70 mg to 280 mg at an interval of every two weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

In various embodiments, the antibody or antibody variant is administered every 4 weeks.

In various embodiments, the antibody or antibody variant is administered at a dose of 70 mg, at a dose of 210 mg or at a dose of 280 mg every 2 weeks or every 4 weeks.

The disclosure also provides a method for treating asthma in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

The disclosure further provides a method for treating asthma in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

In various embodiments, the anti-TSLP antibody variant has substantially similar pK characteristics as tezepelumab in humans.

In various embodiments, the antibody or antibody variant is administered for a period of at least 4 months, 6 months, 9 months, 1 year or more.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

In one embodiment, the anti-TSLP antibody variant is selected from the group consisting of a diabody, a triabody, a tetrabody, a Fab fragment, a single domain antibody, an scFv, wherein the dose is adjusted such that the binding sites are equimolar to those dosed by bivalent antibodies.

In various embodiments, the antibody is an IgG2 antibody.

In one embodiment, the antibody or antibody variant is a human antibody.

In various embodiments, the antibody is tezepelumab. In various embodiments, the tezepelumab is an IgG2 antibody having the full length heavy and light chain amino acid sequences set out in SEQ ID NOs: 105 and 106, respectively.

In various embodiments, the antibody or antibody variant further comprises a pharmaceutically acceptable carrier or excipient.

In various embodiments, the asthma is severe asthma. It is further contemplated that the asthma is eosinophilic or non-eosinophilic asthma, optionally the asthma is low eosinophil asthma.

Data presented herein demonstrates an anti-TSLP antibody that substantially affects two important markers of inflammation of asthma: blood eosinophil counts and the fraction of exhaled nitric oxide. The data show that an anti-TSLP antibody reduces the level of both inflammatory markers, reduces the asthma exacerbation rate, improves lung function irrespective of asthma phenotype (eosinophilic (allergic and nonallergic) and noneosinophilic/low eosinophilic asthma), and blocks at least two important inflammatory pathways in asthma. The anti-TSLP antibody, therefore, is able to treat a patient having either asthma phenotype: eosinophilic (allergic and nonallergic) or noneosinophilic/low eosinophilic asthma. Accordingly, provided herein is a method of treating a patient having low eosinophil asthma comprising administering an anti-TSLP antibody as described herein. Also contemplated is a method for treating a subject having asthma characterized by a low Th2 profile comprising administering an anti-TSLP antibody. In various embodiments, the antibody is tezepelumab or another anti-TSLP antibody described in the art. Exemplary antibodies are described further in the Detailed Description.

In various embodiments, the subject is an adult. In various embodiments, the subject is a child or adolescent.

It is contemplated that administration of the anti-TSLP antibody or antibody variant decreases eosinophils in blood, sputum, broncheoalveolar fluid, or lungs of the subject.

It is further contemplated that administration of the anti-TSLP antibody or antibody variant shifts cell counts in the subject from a Th2 high population to a Th2 low population.

In various embodiments, administration of the anti-TSLP antibody or antibody variant improves one or more measures of asthma in a subject selected from the group consisting of forced expiratory volume (FEV), FEV₁ reversibility, forced vital capacity (FVC), FeNO, Asthma Control Questionnaire-6 score and AQLQ(S)+12 score.

In one embodiment, the administration improves one or more symptoms of asthma as measured by an asthma symptom diary.

Further provided is a method for treating asthma in a subject comprising administering a therapeutically effective amount of an anti-TSLP antibody or antibody variant in a dose of 70 to 280 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2, wherein the antibody is an IgG2 antibody.

In various embodiments, the IgG2 the antibody is administered every 2 weeks or every 4 weeks.

In various embodiments, the IgG2 antibody is administered at a dose of 70 mg, 210 mg or 280 mg every 2 weeks or every 4 weeks.

Also provided is a method of reducing the frequency of asthma exacerbation in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

Further contemplated is a method of reducing the frequency of asthma exacerbation in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

It is contemplated that the dosing and antibody and antibody variant types referenced above apply to each method contemplated herein.

In various embodiments, the antibody or antibody variant further comprises a pharmaceutically acceptable carrier or excipient.

In various embodiments, the administration delays the time to an asthma exacerbation compared to a subject not receiving the anti-TSLP antibody.

In various embodiments, the administration reduces frequency of or levels of co-administered therapy in the subject. Optionally, the co-administered therapy is inhaled corticosteroids (ICS), long-acting β2 agonist (LABA), leukotriene receptor antagonists (LTRA), long-acting anti-muscarinics (LAMA), cromones, short-acting β2 agonist (SABA), and theophylline or oral corticosteroids.

In various embodiments, the administration eliminates the need for corticosteroid therapy.

In various embodiments, the administration is subcutaneous or intravenous.

Also provided herein is a method of treating chronic obstructive pulmonary disease (COPD) comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

Also provided is a method of treating chronic obstructive pulmonary disease (COPD) in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

Also provided herein is a method for reducing ACQ-6 score in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

Further provided is a method for reducing ACQ-6 score in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

Provided herein is a method for reducing ACQ-6 score in a subject having a low eosinophil profile comprising administering a therapeutically effective amount of an anti-TSLP antibody or antibody variant, wherein the antibody or antibody variant binding to TSLP inhibits TSLP activity. Also provided is a method for reducing ACQ-6 score in a subject having a Th2 low profile comprising administering a therapeutically effective amount of an anti-TSLP antibody or antibody variant, wherein the antibody or antibody variant binding to TSLP inhibits TSLP activity.

Also contemplated is a method for treating asthma in a subject, including severe asthma, eosinophilic or non-eosinophilic asthma and low eosinophil asthma comprising administering a therapeutically effective amount of an anti-TSLP antibody or antibody variant, wherein the antibody or antibody variant binding to TSLP inhibits TSLP activity.

In various embodiments, the subject has an eosinophil count less than 250 cells/μL at start of treatment.

Also provided is a method for treating asthma in a subject having a Th2 low profile comprising administering a therapeutically effective amount of an anti-TSLP antibody or antibody variant, wherein the antibody or antibody variant binding to TSLP inhibits TSLP activity.

In various embodiments, the subject has a Th2 profile of IgE less than or equal to 100 IU/ml or eosinophil count of less than 140 cells/μL at the time of diagnosis.

In various embodiments, the antibody is tezepelumab or another anti-TSLP antibody described in the art, e.g., in Table A. Exemplary antibodies are described further in the Detailed Description.

### BRIEF DESCRIPTION OF THE DRAWINGS

FIGS. 1A-1D show the effects of antibody treatment at the different doses in various measures of asthma symptoms. FIG. 1A, asthma exacerbation rate; FIG. 1B, changes from baseline in the postbronchodilator FEV1; FIG. 1C, change from baseline in ACQ-6; FIG. 1D, change from baseline in AQLQ score.

FIGS. 2A-2B show the effects of antibody treatment in patients receiving glucocorticoids. FIG. 2A: Lines within the squares represent the median, the diamond symbol represents the mean, the boxes represent the 25^{th} to 75^{th} percentile and the whiskers represent the range (highest and lowest value). FIG. 2B: Histogram of baseline inhaled glucocorticoid dose (fluticasone equivalents).

FIG. 3 shows a Kaplan-Meier Curve for Time to First Asthma Exacerbation through Week 52 in the Intention-to-Treat population. *P-values are nominal and without multiplicity adjustment

FIGS. 4A-4B show the change from baseline in peripheral blood eosinophils (cell/μl) (FIG. 4A), and total IgE (IU/ml) (FIG. 4B), over time in the Intention-to-Treat population.

FIG. 5 shows the change from baseline in the fraction of exhaled nitric oxide (FENO) in treated subjects.

FIGS. 6A-6B show annualized rate of asthma exacerbations, according to Baseline Biomarker Status at Week 52 (FIG. 6A), and change from baseline in the fraction of exhaled nitric oxide (FENO) (FIG. 6B). In FIG. 6A, nominal two-sided P values of less than 0.05 for the comparison with the placebo group are shown. A clinically meaningful cutoff of 24 ppb was used for the FeNO subpopulation analysis. A high status with respect to type 2 helper T (Th2) cells was defined as an IgE level of more than 100 IU per milliliter and a blood eosinophil count of 140 cells or more per microliter; a low Th2 status was defined as an IgE level of 100 IU or less per milliliter or a blood eosinophil count of less than 140 cells per microliter.

FIG. 7 (Table 1A) describes subject inclusion and exclusion criteria.

FIG. 8 (Table 1B) describes baseline demographics and clinical characteristics in the Intention-To-Treat population.

FIG. 9 (Table 2) shows annualized asthma exacerbation rate reduction, and change from baseline in FEV1, ACQ and AQLQ in the eosinophil sub-populations <250 cells/μl and ≥250 cells/μl.

FIG. 10 (Table 3) shows change from baseline in ACQ-6 (week 50) and AQLQ(S)+12 (week 48) in the Intention-to-Treat population.

FIG. 11 (Table 4) shows the annualized asthma exacerbation rate reduction and change from baseline in FEV1 (week 52), ACQ-6 (week 50), and AQLQ(S)+12 (week 48) in patient sub-populations: Th2 status, serum periostin.

FIG. 12 (Table 5) shows the annualized asthma exacerbation rate reduction and change from baseline in FEV₁ (week 52), ACQ-6 (week 50), and AQLQ(S)+12 (week 48) in patient sub-populations: FENO, allergic status, current post-BD reversibility.

FIG. 13 (Table 6) shows the change from baseline in post-BD FEV₁ and pre- and post-BD forced vital capacity at week 52 in the Intention-To-Treat population.

FIG. 14 (Table 7) shows the annualized rate of severe asthma exacerbations, time to first asthma exacerbation/severe asthma exacerbation, and proportion of patients with one or more asthma exacerbation at week 52 in the Intention-To-Treat population.

FIG. 15 (Table 8) is a post-hoc analysis of annualized asthma exacerbation rate reduction stratified by blood eosinophil count <400 cells/μl vs≥400 cells/μl through week 52.

FIG. 16 (Table 9) shows the annualized asthma exacerbation rate reduction stratified by Patients on a medium- or high-dose of inhaled glucocorticoid and by patients on maintenance oral glucocorticoids through week 52.

FIG. 17 (Table 10) shows annualized asthma exacerbation rate reduction stratified by number of prior asthma exacerbations and by smoking history* through week 52.

FIG. 18 (Table 11) shows the change from baseline in Medimmune ASMA score at week 52.

FIG. 19 (Table 12) shows all treatment-emergent serious adverse events in the as-treated population.

### DETAILED DESCRIPTION

Use of an anti-TSLP antibody addresses an unmet need in asthma patients in which other medications may not control moderate to severe asthma. For example anti-TSLP antibody tezepelumab might reduce exacerbations in both in eosinophil (EOS)-low and high in EOS-high patients. It is further contemplated that treatment with tezepelumab could eliminate daily disease activity and make more patients steroid-free or reduce the need for use of steroids in the treatment of asthma.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs. The following references provide one of skill with a general definition of many of the terms used in this disclosure include, but are not limited to: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “asthma” as used herein refers to allergic, non-allergic, eosinophilic, and non-eosinophillic asthma.

The term “allergic asthma” as used herein refers to asthma that is triggered by one or more inhaled allergens. Such patients have a positive IgE fluorescence enzyme immunoassay (FEIA) level to one or more allergens that trigger an asthmatic response.

Typically, most allergic asthma is associated with Th2-type inflammation.

The term “non-allergic asthma” refers to patients that have low eosinophil, low Th2, or low IgE at the time of diagnosis. A patient who has “non-allergic asthma” is typically negative in the IgE fluorescence enzyme immunoassay (FEIA) in response to a panel of allergens, including region-specific allergens. In addition to low IgE, those patients often have low or no eosinophil counts and low Th2 counts at the time of diagnosis.

The term “severe asthma” as used herein refers to asthma that requires high intensity treatment (e.g., GINA Step 4 and Step 5) to maintain good control, or where good control is not achieved despite high intensity treatment (GINA, Global Strategy for Asthma Management and Prevention. Global Initiative for Asthma (GINA) December 2012).

The term “eosinophilic asthma” as used herein refers to an asthma patient having a screening blood eosinophil count of ≥250 cells/μL. “Low eosinophilic” asthma refers to asthma patients having less than 250 cells/uL blood or serum.

The term “Th2-type inflammation” as used herein refers to a subject having a screening blood eosinophil count 140 cells/μL and a screening total serum IgE level of >100 IU/mL (Corren et al, N Engl J Med. 22; 365(12):1088-98, 2011). A “Th2 high” asthma population or profile refers to a subject having IgE>100 IU/mL and Blood Eosinophil Count ≥140 cells/μL. A “Th2 low” asthma population refers to a subject having IgE<100 IU/mL and Blood Eosinophil Count ≥140 cells/μL

An “elevated FeNO” (Fractional exhaled nitric oxide) as used herein refers to a baseline FeNO measurement greater than or equal to the median from all randomized subjects in the study. Elevated FeNO refers to FeNO levels of 24 or above.

The term “elevated serum periostin level” as used herein refers to a patient having a baseline serum periostin level greater than or equal to the median from all randomized subjects in the study. Periostin has been shown to be involved in certain aspects of allergic inflammation, including eosinophil recruitment, airway remodeling, and development of a Th2 phenotype (Li et al., Respir Res. 16(1):57, 2015).

The term “current post-bronchodilator (BD) forced expiratory volume in 1 second (FEV₁) reversibility” as used herein refers to a post-BD change in FEV₁ of ≥12% and 200 mL

The term “asthma exacerbation” as used herein refers to a worsening of asthma that leads to any of the following: Use of systemic corticosteroids for at least 3 days; a single depo-injectable dose of corticosteroids is considered equivalent to a 3-day course of systemic corticosteroids; for subjects receiving maintenance OCS, a temporary doubling of the maintenance dose for at least 3 days qualifies; an ED visit due to asthma that required systemic corticosteroids (as per above); an inpatient hospitalization due to asthma. Additional measures associated with asthma exacerbations are also being examined to determine effect. These include hospitalizations related to asthma exacerbations (i.e., severe asthma exacerbations), time to first asthma exacerbation, and the proportion of subjects with one or more asthma exacerbation/severe asthma exacerbation.

The term “worsening of asthma” refers to new or increased symptoms and/or signs (examination or lung function) that can be either concerning to the subject (subject-driven) or related to an Asthma Daily Diary alert (diary-driven) via the ePRO device. Asthma-worsening thresholds include: decrease in morning peak flow ≥30% on at least 2 of 3 successive days compared with baseline (last 7 days of run-in), and/or a ≥50% increase in rescue medication (minimum increase of 2 or more puffs, or one new or additional nebulized β2 agonist) on at least 2 of 3 successive days compared with the average use for the previous week, and/or nocturnal awakening due to asthma requiring rescue medication use for at least 2 of 3 successive nights, and/or an increase in total asthma symptom score (the sum of daytime [evening assessment] and nighttime [morning assessment]) of at least 2 units above the screening/run-in period average (last 10 days of screening/run-in), or the highest possible score (daily score of 6), on at least 2 of 3 successive days.

The term “cytokine” as used herein refers to one or more small (5-20 kD) proteins released by cells that have a specific effect on interactions and communications between cells or on the behavior of cells, such as immune cell proliferation and differentiation. Functions of cytokines in the immune system include, promoting influx of circulating leukocytes and lymphocytes into the site of immunological encounter; stimulating the development and proliferation of B cells, T cells, peripheral blood mononuclear cells (PBMCs) and other immune cells; and providing antimicrobial activity. Exemplary immune cytokines, include but are not limited to, IL-1, IL-2, IL-3, IL-4, IL-5, IL-6, IL-7, IL-9, IL-10, IL-12, IL-13, IL-15, IL17A, IL-17F, IL-18, IL-21, IL-22, interferon (including IFN alpha, beta, and gamma), tumor necrosis factor (including TNF alpha, beta), transforming growth factor (including TGF alpha, beta), granulocyte colony stimulating factor (GCSF), granulocyte macrophage colony stimulating factor (GMCSF) and thymic stromal lymphopoietin (TSLP).

A “T helper (Th) 1 cytokine” or “Th1-specific cytokine” refers to cytokines that are expressed (intracellularly and/or secreted) by Th1 T cells, and include IFN-g, TNF-α, and IL-12. A “Th2 cytokine” or “Th2-specific cytokine” refers to cytokines that are expressed (intracellularly and/or secreted) by Th2 T cells, including IL-4, IL-5, IL-13, and IL-10. A “Th17 cytokine” or “Th17-specific cytokine” refers to cytokines that are expressed (intracellularly and/or secreted) by Th17 T cells, including IL-17A, IL-17F, IL-22 and IL-21. Certain populations of Th17 cells express IFN-g and/or IL-2 in addition to the Th17 cytokines listed herein. A polyfunctional CTL cytokine includes IFN-g, TNF-α, IL-2 and IL-17.

The term “specifically binds” is “antigen specific”, is “specific for”, “selective binding agent”, “specific binding agent”, “antigen target” or is “immunoreactive” with an antigen refers to an antibody or polypeptide that binds an target antigen with greater affinity than other antigens of similar sequence. It is contemplated herein that the agent specifically binds target proteins useful in identifying immune cell types, for example, a surface antigen (e.g., T cell receptor, CD3), a cytokine (e.g., TSLP, IL-4, IL-5, IL-13, IL-17, IFN-g, TNF-α) and the like. In various embodiments, the antibody specifically binds the target antigen, but can cross-react with an ortholog of a closely related species, e.g. an antibody may being human protein and also bind a closely related primate protein.

The term “antibody” or “immunoglobulin” refers to a tetrameric glycoprotein that consists of two heavy chains and two light chains, each comprising a variable region and a constant region. “Heavy Chains” and “Light Chains” refer to substantially full length canonical immunoglobulin light and heavy chains (see e.g., Immunobiology, 5th Edition (Janeway and Travers et al., Eds., 2001). Antigen-binding portions may be produced by recombinant DNA techniques or by enzymatic or chemical cleavage of intact antibodies. The term “antibody” includes monoclonal antibodies, polyclonal antibodies, chimeric antibodies, human antibodies, and humanized antibodies.

Antibody variants include antibody fragments and anti-body like proteins with changes to structure of canonical tetrameric antibodies. Typically antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

Antibody fragments include antigen-binding portions of the antibody including, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Valency” refers to the number of antigen binding sites on each antibody or antibody fragment that targets an epitope. A typical full length IgG molecule, or F(ab)₂ is “bivalent” in that it has two identical target binding sites. A “monovalent’ antibody fragment such as a F(ab)′ or scFc with a single antigen binding site. Trivalent or tetravalent antigen binding proteins can also be engineered to be multivalent.

“Monoclonal antibody” refers to an antibody obtained from a population of substantially homogeneous antibodies, i.e., the individual antibodies comprising the population are identical except for possible naturally occurring mutations that may be present in minor amounts.

The term “inhibits TSLP activity” includes inhibiting any one or more of the following: binding of TSLP to its receptor; proliferation, activation, or differentiation of cells expressing TSLPR in the presence of TSLP; inhibition of Th2 cytokine production in a polarization assay in the presence of TSLP; dendritic cell activation or maturation in the presence of TSLP; and mast cell cytokine release in the presence of TSLP. See, e.g., U.S. Pat. No. 7,982,016 B2, column 6 and example 8 and US 2012/0020988 A1, examples 7-10.

The term “sample” or “biological sample” refers to a specimen obtained from a subject for use in the present methods, and includes urine, whole blood, plasma, serum, saliva, sputum, tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic agent in an amount and for a time sufficient to induce a sustained improvement over baseline of an indicator that reflects the severity of the particular disorder.

The term “therapeutically effective amount” refers to an amount of therapeutic agent that is effective to ameliorate or lessen symptoms or signs of disease associated with a disease or disorder.

### Asthma

Asthma is a chronic inflammatory disorder of the airways. Each year, asthma accounts for an estimated 1.1 million outpatient visits, 1.6 million emergency room visits, 444,000 hospitalizations (Defrances et al, 2008) Available at: http://www.cdc.gov/nchs/data/nhsr/nhsr005.pdf, and 3,500 deaths in the U.S. In susceptible individuals, asthmatic inflammation causes recurrent episodes of wheezing, breathlessness, chest tightness, and cough. The etiology of asthma is thought to be multi-factorial, influenced by both genetic environmental mechanisms,^{1,2} with environmental allergens an important cause.^{2,3} The majority of cases arise when a person becomes hypersensitive to allergens (atopy). Atopy is characterized by an increase in Th2 cells and Th2 cytokine expression and IgE production. Approximately 10 million patients in the United States are thought to have allergy-induced asthma. Despite the available therapeutic options, asthma continues to be a major health problem. Worldwide, asthma currently affects approximately 300 million people; by 2020, asthma is expected to affect 400 million people (Partridge, Eur Resp Rev. 16:67-72, 2007).

Allergen inhalation by atopic asthmatics induces some of the manifestations of asthma, including reversible airflow obstruction, airway hyperresponsiveness, and eosinophilic and basophilic airway inflammation. Allergen inhalation challenge has become the predominant model of asthma in many species (Bates et al., Am J Physiol Lung Cell Mol Physiol. 297(3):L401-10, 2009; Diamant et al., J Allergy Clin Immunol. 132(5):1045-1055, 2013.)

Different asthma subtypes that are refractory to steroid treatment have been identified. Eosinophils are important inflammatory cells in allergic asthma that is characteristically mediated by Th2-type CD4+ T cells. Neutrophilic airway inflammation is associated with corticosteroid treatment in severe asthma and can be mediated by Th1- or Th17-type T cells (Mishra et al., Dis. Model. Mech. 6:877-888, 2013).

Measures of diagnosis and assessment of asthma include the following:

Airway inflammation evaluated using a standardized single-breath Fraction of Exhaled Nitric Oxide (FeNO)(American Thoracic Society; ATS, Am J Respir Crit Care Med. 171(8):912-30, 2005) test. For example, subjects inhale to total lung capacity through the NIOX MINO® Airway Inflammation Monitor and then exhale for 10 seconds at 50 mL/sec (assisted by visual and auditory cues).

Spirometry is performed according to ATS/European Respiratory Society (ERS) guidelines (Miller et al, Eur Respir J. 26(1):153-61, 2005). For example, multiple forced expiratory efforts (at least 3 but no more than 8) is performed at each spirometry session and the 2 best efforts that meet ATS/ERS acceptability and reproducibility criteria are recorded. The best efforts will be based on the highest FEV₁. The maximum FEV₁ of the 2 best efforts will be used for the analysis. Both the absolute measurement (for FEV₁ and FVC) and the percentage of predicted normal value will be recorded using appropriate reference values. The highest FVC will also be reported regardless of the effort in which it occurred (even if the effort did not result in the highest FEV₁).

Post-bronchodilator (Post-BD) spirometry testing is assessed after the subject has performed pre-BD spirometry. Maximal bronchodilation is induced using a SABA such as albuterol (90 μg metered dose) or salbutamol (100 μg metered dose) or equivalent with a spacer device for a maximum of 8 total puffs (Sorkness et al, J Appl Physiol. 104(2):394-403, 2008). The highest pre- and post-BD FEV₁ obtained after 4, 6, or 8 puffs is used to determine reversibility and for analysis. Reversibility algorithm is as follows:

% Reversibility=(post-BD FEV1−pre-BD FEV1)×100/pre-BD FEV1

Home peak flow testing for peak expiratory flow rate (PEFR) is performed twice daily, in the morning upon awakening and in the evening prior to bedtime using a peak flow meter from the morning of Visit 2 (Week—4) through Week 64. When possible, ambulatory lung function measurements should be taken at least 6 hours after the last dose of SABA rescue medication.

The Asthma Daily Diary includes the following daily assessments: asthma symptoms; inhalations of rescue medication; nighttime awakening due to asthma requiring rescue medication use, asthma-related activity limitations, asthma-related stress, and background medication compliance. The Asthma Daily Diary is completed each morning and evening. There will be triggers in the ePRO device to alert the subjects to signs of worsening of asthma.

The Asthma Control Questionnaire (ACQ) 6 is a patient-reported questionnaire assessing asthma symptoms (i.e., night-time waking, symptoms on waking, activity limitation, shortness of breath, wheezing) and daily rescue bronchodilator use and FEV₁ (Juniper et al, October 1999). The ACQ-6 is a shortened version of the ACQ that omits the FEV₁ measurement from the original ACQ score. Questions are weighted equally and scored from 0 (totally controlled) to 6 (severely uncontrolled). The mean ACQ score is the mean of the responses. Mean scores of s 0.75 indicate well-controlled asthma, scores between 0.75 and ≤1.5 indicate partly-controlled asthma, and a score >1.5 indicates uncontrolled asthma (Juniper et al, Respir Med. 100(4):616-21, 2006). Individual changes of at least 0.5 are considered to be clinically meaningful (Juniper et al, Respir Med. 99(5):553-8, 2005).

The Asthma Quality of Life Questionnaire, Standardized (AQLQ[S])+12 (AQLQ(S)+12) is a 32-item questionnaire that measures the HRQoL experienced by asthma patients (Juniper et al, Chest. 115(5):1265-70, May 1999). The questionnaire comprises 4 separate domains (symptoms, activity limitations, emotional function, and environmental stimuli). Subjects are asked to recall their experiences during the previous 2 weeks and to score each of the 32 questions on a 7-point scale ranging from 7 (no impairment) to 1 (severe impairment). The overall score is calculated as the mean response to all questions. The 4 individual domain scores (symptoms, activity limitations, emotional function, and environmental stimuli) are the means of the responses to the questions in each of the domains. Individual improvement in both the overall score and individual domain scores of 0.5 has been identified as a minimally important change, with score changes of ≥1.5 identified as large meaningful changes (Juniper et al, J Clin Epidemiol. 47(1):81-7, 1994).

### TSLP

Thymic stromal lymphopoietin (TSLP) is an epithelial cell-derived cytokine that is produced in response to pro-inflammatory stimuli and drives allergic inflammatory responses primarily through its activity on dendritic cells (Gilliet, J Exp Med. 197:1059-1067, 2003; Soumelis, Nat Immunol. 3:673-680, 2002; Reche, J Immunol. 167:336-343, 2001), mast cells (Allakhverdi, J Exp Med. 204:253-258, 2007) and CD34+ progenitor cells.⁹ TSLP signals through a heterodimeric receptor consisting of the interleukin (IL)-7 receptor alpha (IL-7Rα) chain and a common γ chain-like receptor (TSLPR) (Pandey, Nat Immunol. 1:59-64, 2000; Park, J Exp Med. 192:659-669, 2000).

Human TSLP mRNA^{10,11} and protein levels¹¹ are increased in the airways of asthmatic individuals compared to controls, and the magnitude of this expression correlates with disease severity.¹⁰ Recent studies have demonstrated association of a single nucleotide polymorphism in the human TSLP locus with protection from asthma, atopic asthma and airway hyperresponsiveness, suggesting that differential regulation of TSLP gene expression might influence disease susceptibility.^{1,12,13} These data suggest that targeting TSLP may inhibit multiple biological pathways involved in asthma.

Earlier non-clinical studies of TSLP suggested that after TSLP is released from airway epithelial cells or stromal cells, it activates mast cells, dendritic cells, and T cells to release Th2 cytokines (e.g., IL-4/13/5). Recently published human data demonstrated a good correlation between tissue TSLP gene and protein expression, a Th2 gene signature score, and tissue eosinophils in severe asthma. Therefore, an anti-TSLP target therapy may be effective in asthmatic patients with Th2-type inflammation (Shikotra et al, J Allergy Clin Immunol. 129(1):104-11, 2012).

Data from other studies suggest that TSLP may promote airway inflammation through Th2 independent pathways such as the crosstalk between airway smooth muscle and mast cells (Allakhverdi et al, J Allergy Clin Immunol. 123(4):958-60, 2009; Shikotra et al, supra). TSLP can also promote induction of T cells to differentiate into Th-17-cytokine producing cells with a resultant increase in neutrophilic inflammation commonly seen in more severe asthma (Tanaka et al, Clin Exp Allergy. 39(1):89-100, 2009). These data and other emerging evidence suggest that blocking TSLP may serve to suppress multiple biologic pathways including but not limited to those involving Th2 cytokines (IL-4/13/5).

### Antibodies

It is contemplated that antibodies or antibody variants specific for TSLP are useful in the treatment of asthma, including severe asthma, eosinophlic asthma, no-eosinophilic/low-eosinophilic and other forms of asthma described herein.

Specific binding agents such as antibodies and antibody variants or fragments that bind to their target antigen, e.g., TSLP, are useful in the methods of the invention. In one embodiment, the specific binding agent is an antibody. The antibodies may be monoclonal (MAbs); recombinant; chimeric; humanized, such as complementarity-determining region (CDR)-grafted; human; antibody variants, including single chain; and/or bispecific; as well as fragments; variants; or derivatives thereof. Antibody fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Also encompassed by the invention are human antibodies and antibody variants (including antibody fragments) that bind TSLP. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein.

Chimeric, CDR grafted, and humanized antibodies and/or antibody variants are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

Antibodies and antibody variants (including antibody fragments) useful in the present methods comprise an anti-TSLP antibody comprising a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and

b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody or antibody variant specifically binds to a TSLP polypeptide as set forth in amino acids 29-159 of SEQ ID NO:2.

Also contemplated is an antibody or antibody variant comprising a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and

b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody or antibody variant specifically binds to a TSLP polypeptide as set forth in amino acids 29-159 of SEQ ID NO:2.

Tezepelumab is an exemplary anti-TSLP antibody having: a. i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8;

Tezepelumab also comprises a light chain variable domain having the amino acid sequence set out in SEQ ID NO:12; encoded by a polynucleotide sequence set out in SEQ ID NO:11; and a heavy chain variable domain having the amino acid sequence set out in SEQ ID NO:10, encoded by a polynucleotide sequence set out in SEQ ID NO:9.

Tezepelumab is an IgG2 antibody. The sequence of the full length heavy chain and light chain of tezepelumab, including the IgG2 chain, is set out in SEQ ID NOs: 105 and 106, respectively.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

In various embodiments, the anti-TSLP antibody variant is selected from the group consisting of a diabody, a triabody, a tetrabody, a Fab fragment, single domain antibody, scFv, wherein the dose is adjusted such that the binding sites to be equimolar to the those dosed by bivalent antibodies.

It is contemplated that the antibody or antibody variant is an IgG2 antibody. Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production.

In certain embodiments, derivatives of antibodies include tetrameric glycosylated antibodies wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human TSLP, or to increase or decrease the affinity of the antibodies to human TSLP described herein.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

### Methods of Administration

In one aspect, methods of the present disclosure include a step of administering a therapeutic anti-TSLP antibody or antibody variant described herein, optionally in a pharmaceutically acceptable carrier or excipient. In certain embodiments, the pharmaceutical composition is a sterile composition.

Contemplated herein are methods method for treating asthma in a subject, including severe asthma, eosinophilic or non-eosinophilic asthma and low eosinophil asthma. Surprisingly, it was found herein that treatment with an anti-TSLP antibody is effective at reducing asthma symptoms in a no eosinophil/low eosinophil population as it is in a high eosinophil population. Also contemplated is a method of reducing the frequency of asthma exacerbation in a subject.

Also contemplated herein are methods of treating asthma in a subject having a Th2 high asthma profile or a Th2 low asthma profile. It is contemplated that a TSLP antagonist that inhibits binding of the TSLP protein to its receptor complex will effectively treat a low eosinophil asthma population as the antibody described herein. Similarly, it is contemplated that a TSLP antagonist that inhibits binding of TSLP to its receptor complex will be effective in treating Th2 low asthma populations.

Provided herein is a method of treating a patient having low eosinophil asthma comprising administering an anti-TSLP antibody. Also contemplated is a method for treating a subject having asthma characterized by a low Th2 profile comprising administering an anti-TSLP antibody. In various embodiments, the antibody is tezepelumab or another anti-TSLP antibody described in the art. Exemplary anti-TSLP antibodies include antibodies described in WO 2017/042701, WO 2016/142426, WO 2010/017468, US20170066823, US20120020988 and U.S. Pat. No. 8,637,019, incorporated herein by reference, some of which are described below in Table A. In exemplary aspects, the anti-TSLP antibody is selected from an antibody of Table A.

TABLE A

| | |
|---|---|
| WO2017/042701 | An anti-TSLP antibody comprising a heavy chain (HC) CDR1 comprising |
| | the sequence of SEQ ID NO: 13, a HC CDR2 comprising the sequence of |
| | SEQ ID NO: 14, and a HC CDR3 comprising the sequence of SEQ ID NO: 15; |
| | An anti-TSLP antibody comprising a light chain (LC) CDR1 comprising the |
| | sequence of SEQ ID NO: 16, a LC CDR2 comprising the sequence of |
| | SEQ ID NO: 17, a LC CDR3 comprising the sequence of SEQ ID NO: 18; |
| | An anti-TSLP antibody comprising a heavy chain (HC) CDR1 comprising |
| | the sequence of SEQ ID NO: 19, a HC CDR2 comprising the sequence of |
| | SEQ ID NO: 20, a HC CDR3 comprising the sequence of SEQ ID NO: 15; |
| | An anti-TSLP antibody comprising a light chain (LC) CDR1 comprising the |
| | sequence of SEQ ID NO: 21, a LC CDR2 comprising the sequence of |
| | SEQ ID NO: 22, a LC CDR3 comprising the sequence of SEQ ID NO: 23; |
| | An anti-TSLP antibody comprising a HC variable region comprising the |
| | sequence of SEQ ID NO: 26 and/or a LC variable region comprising the |
| | sequence of SEQ ID NO: 27; |
| | An anti-TSLP antibody comprising a HC variable region comprising the |
| | sequence of SEQ ID NO: 28 and/or a LC variable region comprising the |
| | sequence of SEQ ID NO: 29; |
| | An anti-TSLP antibody that comprises a paratope comprising at least one |
| | of the following residues: Thr28, Asp31, Tyr32, Trp33, Asp56, Glu101, |
| | Ile102, Tyr103, Tyr104, Tyr105 of a heavy chain sequence of SEQ ID NO: |
| | 26 or Gly28, Ser29, Lys30, Tyr31, Tyr48, Asp50, Asn51, Glu52, Asn65, |
| | and Trp92 of a light chain sequence of SEQ ID NO: 27; |
| | An anti-TSLP antibody that specifically binds an epitope in human TSLP, |
| | wherein the epitope comprises at least one of the following residues: |
| | Lys38, Ala41, Leu44, Ser45, Thr46, Ser48, Lys49, Ile52, Thr53, Ser56, |
| | Gly57, Thr58, Lys59, Lys101, Gln145, and Arg149 of SEQ ID NO: 30; |
| WO2016/142426 | An anti-TSLP antibody comprising the amino acid sequence of SEQ ID NO: 31; |
| | An anti-TSLP antibody comprising a CDR1 comprising the sequence of |
| | SEQ ID NO: 32; a CDR2 comprising the sequence of SEQ ID NO: 33, and |
| | a CDR3 comprising the sequence of SEQ ID NO: 34; |
| | An anti-TSLP antibody comprising a CDR1 comprising the sequence of |
| | SEQ ID NO: 32; a CDR2 comprising the sequence of SEQ ID NO: 35, and |
| | a CDR3 comprising the sequence of SEQ ID NO: 34; |
| | An anti-TSLP antibody comprising a variant of the CDR1 of SEQ ID NO: |
| | 31 wherein the residue corresponding to residue 28 in SEQ ID NO: 31 is |
| | Pro, the residue corresponding to residue 30 in SEQ ID NO: 31 is Arg, the |
| | residue corresponding to residue 31 in SEQ ID NO: 31 is Asn, the residue |
| | corresponding to residue 32 in SEQ ID NO: 31 is Trp and the residue |
| | corresponding to residue 34 in SEQ ID NO: 31 is Asp; |
| | An anti-TSLP antibody comprising a variant of the CDR2 of SEQ ID NO: |
| | 31 wherein the residue corresponding to residue 50 in SEQ ID NO: 31 is |
| | Gly, the residue corresponding to residue 53 in SEQ ID NO: 31 is His and |
| | the residue corresponding to residue 55 in SEQ ID NO: 31 is Gln; |
| | An anti-TSLP antibody comprising a variant of the CDR3 of SEQ ID NO: |
| | 31 wherein the residue corresponding to residue 91 in SEQ ID NO: 31 is |
| | He, Leu, Val or Phe, the residue corresponding to residue 92 in SEQ ID |
| | NO: 31 is Gly or Ala, the residue corresponding to residue 93 in SEQ ID |
| | NO: 31 is Glu, Phe, Asp or Ser and the residue corresponding to residue |
| | 94 in SEQ ID NO: 31 is Asp. |
| WO2010/017468 | An anti-TSLP antibody (9B7) comprising a HC CDR3 comprising the |
| | sequence of SEQ ID NO: 38, wherein the other CDRs of the HC and LC |
| | comprise the sequences of SEQ ID NOs: 36, 37, and 39-41; |
| | An anti-TSLP antibody (6C5) comprising a HC CDR3 comprising the |
| | sequence of SEQ ID NO: 44, wherein the other CDRs of the HC and LC |
| | comprise the sequences of SEQ ID NOs: 42, 43, and 45-47; |
| | An anti-TSLP antibody (6A3) comprising a HC CDR3 comprising the |
| | sequence of SEQ ID NO: 50, wherein the other CDRs of the HC and LC |
| | comprise the sequences of SEQ ID NOs: 48, 49, and 51-53; |
| | An anti-TSLP antibody (1A11) comprising a HC CDR3 comprising the |
| | sequence of SEQ ID NO: 56, wherein the other CDRs of the HC and LC |
| | comprise the sequences of SEQ ID NOs: 54, 55, and 57-59; |
| | An anti-TSLP antibody comprising (i) heavy chain variable region of SEQ |
| | ID NO: 60 and/or the light chain variable region of SEQ ID NO: 61; |
| | An anti-TSLP antibody comprising (i) heavy chain variable region of SEQ |
| | ID NO: 62 and/or the light chain variable region of SEQ ID NO: 63; |
| | An anti-TSLP antibody comprising (i) heavy chain variable region of SEQ |
| | ID NO: 64 and/or the light chain variable region of SEQ ID NO: 65; |
| | An anti-TSLP antibody comprising (i) heavy chain variable region of SEQ |
| | ID NO: 66 and/or the light chain variable region of SEQ ID NO: 67; |
| | An anti-TSLP antibody comprising (i) heavy chain variable region of SEQ |
| | ID NO: 68 and/or the light chain variable region of SEQ ID NO: 69; |
| | An anti-TSLP antibody comprising a HC CDR selected from the group |
| | consisting of SEQ ID NO: 38, SEQ ID NO: 44, SEQ ID NO: 50 and SEQ ID |
| | NO: 56, and analogs thereof; |
| | An anti-TSLP antibody comprising a heavy chain comprising the following |
| | CDRs or analogs thereof CDRH1: RYNVH (SEQ ID NO: 36), CDRH2: |
| | MIWDGGSTDYNSALKS (SEQ ID NO: 37), CDRH3: NRYESG (SEQ ID |
| | NO: 38), and a light chain comprising the following CDRs or analogs |
| | thereof CDRL1: KSSQSLLNSGNRKNYLT (SEQ ID NO: 39), CDRL2: |
| | WASTRES (SEQ ID NO: 40), and CDRL3: QNDYTYPFTFGS (SEQ ID |
| | NO: 41); or |
| | An anti-TSLP antibody comprising a heavy chain comprising the following |
| | CDRs or analogs thereof CRDH1: AYWMS (SEQ ID NO: 42), CDRH2: |
| | EINPDSSTINCTPSLKD (SEQ ID NO: 43), CDRH3: RLRPFWYFDVW |
| | (SEQ ID NO: 44), and a light chain comprising the following CDRs or |
| | analogs thereof CDRL1: RSSQSIVQSNGNTYLE (SEQ ID NO: 45), |
| | CDRL2: KVSNRFS (SEQ ID NQ: 46), and CDRL3: FQGSHVPRT |
| | (SEQ ID NO: 47); |
| | An anti-TSLP antibody comprising a heavy chain comprising the following |
| | CDRs or analogs thereof CRDH1: TDYAWN (SEQ ID NO: 48), CDRH2: |
| | YIFYSGSTTYTPSLKS (SEQ ID NO: 49), CDRH3: GGYDVNYF (SEQ ID |
| | NO: 50), and a light chain comprising the following CDRs or analogs |
| | thereof CDPL1: LASQTIGAWLA (SEQ ID NO: 51), CDRL2: AATRLAD |
| | (SEQ ID NQ: 52), and CDPL3: QQFFSTPWT (SEQ ID NQ: 53); |
| | An anti-TSLP antibody comprising a heavy chain comprising the following |
| | CDRs or analogs thereof CDRH1: GYTMN (SEQ ID NO: 54), CDRH2: |
| | LINPYNGVTSYNQKFK (SEQ ID NO: 55), CDRH3: GDGNYWYF (SEQ ID |
| | NO: 56), and a light chain comprising the following CDRs or analogs |
| | thereof CDRL1: SASSSVTYMHW (SEQ ID NO: 57), CDRL2: EISKLAS |
| | (SEQ ID NO: 58), and CDRL3: QEWNYPYTF (SEQ ID NO: 59); |
| | An anti-TSLP antibody comprising a HC CDR1 comprising the sequence |
| | of SEQ ID NO: 70; a CDR2 comprising the sequence of SEQ ID NO: 71, |
| | and a CDR3 comprising the sequence of SEQ ID NO: 72; |
| | An anti-TSLP antibody comprising a LC CDR1 comprising the sequence of |
| | SEQ ID NO: 73; a CDR2 comprising the sequence of SEQ ID NO: 74, and |
| | a CDR3 comprising the sequence of SEQ ID NO: 75; |
| US2012/0020988 | An anti-TSLP antibody comprising a heavy chain variable domain |
| | comprising a CDR1 region of SEQ ID NO: 76, a CDR2 region of SEQ ID |
| | NO: 77, and CDR3 region of SEQ ID NO: 78, and a light chain variable |
| | domain comprising a CDR1 region of SEQ ID NO: 79, a CDR2 region of |
| | SEQ ID NO: 80, and a CDR3 region of SEQ ID NO: 81. |
| | An anti-TSLP antibody comprising a heavy chain variable domain |
| | comprising SEQ ID NO: 82 and a light chain variable domain comprising |
| | SEQ ID NO: 83; |
| | An anti-TSLP antibody comprising a heavy chain variable domain |
| | comprising a CDR1 region of SEQ ID NO: 76 or 84, a CDR2 region of |
| | SEQ ID NO: 77 or 85, and CDR3 region of SEQ ID NO: 78, and a light |
| | chain variable domain comprising a CDR1 region of SEQ ID NO: 79 or 86, |
| | a CDR2 region of SEQ ID NO: 80, 87, or 88, and a CDR3 region of SEQ |
| | ID NO: 81. |
| | An anti-TSLP antibody comprising a heavy chain variable domain |
| | comprising a CDR1 region of SEQ ID NO: 76, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 87 and a CDR3 region of SEQ ID NO: 81; |
| | An anti-TSLP antibody comprising a heavy chain variable domain |
| | comprising a CDR1 region of SEQ ID NO: 76, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 88 and a CDR3 region of SEQ ID NO: 81; |
| | An anti-TSLP antibody comprising a heavy chain variable domain |
| | comprising a CDR1 region of SEQ ID NO: 84, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 88 and a CDR3 region of SEQ ID NO: 81; or |
| | An anti-TSLP antibody comprising a heavy chain variable domain |
| | comprising a CDR1 region of SEQ ID NO: 76, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 80 and a CDR3 region of SEQ ID NO: 81. |
| | An anti-TSLP antibody comprising a heavy chain variable domain |
| | comprises SEQ ID NO: 89 and a light chain variable domain comprises |
| | SEQ ID NO: 90; |
| | An anti-TSLP antibody comprising a heavy chain variable domain |
| | comprises SEQ ID NO: 89 and a light chain variable domain comprises |
| | SEQ ID NO: 91; |
| | An anti-TSLP antibody comprising a heavy chain variable domain |
| | comprises SEQ ID NO: 92 and a light chain variable domain comprises |
| | SEQ ID NO: 93; |
| | An anti-TSLP antibody comprising a heavy chain variable domain |
| | comprises SEQ ID NO: 89 and a light chain variable domain comprises |
| | SEQ ID NO: 94, |
| U.S. Pat. No. | An anti-TSLP antibody comprising heavy chain variable region comprising: |
| 8,637,019 | a CDR-H1 sequence comprising SEQ ID NO: 95, a CDR-H2 sequence |
| | comprising SEQ ID NO: 96, and a CDR-H3 sequence comprising SEQ ID |
| | NO: 97; and/or an antibody light chain variable region or a TSLP-binding |
| | fragment thereof, said light chain variable region comprising: a CDR-L1 |
| | sequence comprising SEQ ID NO: 98, a CDR-L2 sequence comprising |
| | SEQ ID NO: 99, and a CDR-L3 sequence comprising SEQ ID NO: 100. |
| | An anti-TSLP antibody comprising a heavy chain variable region |
| | comprises the amino acid sequence of SEQ ID NO: 101 and the light chain |
| | variable region comprises the amino acid sequence of SEQ ID NO: 102. |
| | An anti-TSLP antibody comprising SEQ ID NO: 103 and SEQ ID NO: 104. |

Also contemplated are methods for treating chronic obstructive pulmonary disease (COPD) in a subject comprising administering an anti-TSLP antibody or antibody variant.

It is contemplated that the subject to be treated is human. The subject may be an adult, an adolescent or a child.

Therapeutic antibody (or antibody variant) compositions may be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, monthly, or at a longer interval.

In various embodiments, the amounts of therapeutic agent, such as a bivalent antibody having two TSLP binding sites, in a given dosage may vary according to the size of the individual to whom the therapy is being administered as well as the characteristics of the disorder being treated.

In exemplary treatments, the anti-TSLP antibody or antibody variant is administered in a dose range of about 70 mg to about 280 mg per daily dose. For example, the dose may be given in about 70 mg, 210 mg or 280 mg. In various embodiments, the anti-TSLP antibody or antibody variant may be administered at a dose of 70, 80, 90, 100, 110, 120, 130, 140, 150, 160, 10, 180, 190, 200, 210, 220, 230, 240, 250, 260, 270 or 280 mg per dose. These concentrations may be administered as a single dosage form or as multiple doses. The above doses are given every two weeks or every four weeks. In various embodiments, the anti-TSLP antibody or antibody variant is administered at a single dose of 70 mg every two weeks or every four weeks. In various embodiments, the anti-TSLP antibody or antibody variant is administered at a single dose of 210 mg every two weeks or every four weeks. In various embodiments, the anti-TSLP antibody or antibody variant is administered at a single dose of 280 mg every two weeks or every four weeks.

For antibody variants, the amount of antibody variant should be such that the number of TSLP binding sites that are in the dose have an equimolar number of TSLP binding sites to canonical bivalent antibody described above.

It is contemplated that the anti-TSLP antibody or antibody variant is administered every 2 weeks or every 4 weeks for a period of at least 4 months, 6 months, 9 months, 1 year or more. In various embodiments, the administration is subcutaneous or intravenous.

Treatment with the anti-TSLP antibody or antibody variant is contemplated to decrease eosinophils in blood, sputum, broncheoalveolar fluid, or lungs of the subject. It is also contemplated that the administration shifts cell counts in the subject from a Th2 high population to a Th2 low population. It is further contemplated that administration of the anti-TSLP antibody improves one or more measures of asthma in a subject selected from the group consisting of forced expiratory volume (FEV), FEV1 reversibility, forced vital capacity (FVC), FeNO, Asthma Control Questionnaire-6 score and AQLQ(S)+12 score.

Improvement in asthma may be measured as one or more of the following: reduction in AER (annualized exacerbation rate), reduction in hospitalizations/severe exacerbations for asthma, change from baseline (increase) in time to first asthma exacerbation (following onset of treatment with anti-TSLP antibody), decrease relative to placebo in proportion of subjects with one or more asthma exacerbations or severe exacerbations over the course of treatment, e.g., 52 weeks, change from baseline (increase) in FEV1 and FVC (pre-broncholdilator and post-bronchodilator), change from baseline (decrease) in blood or sputum eosinophils (or lung eosinophils if biopsy or BAL fluid obtained), change from baseline (decrease) in FeNO, change from baseline (decrease) in IgE, improvement in asthma symptoms and control as measured by PROs including ACQ and variants, AQLQ and variants, SGRQ, and asthma symptom diaries, change (decrease) in use of rescue medications, decrease in use of systemic corticosteroids, decrease in Th2/Th1 cell ratio in blood. Most/all these measures should be in total population and subpopulations including hi and low eosinophils (Greater than or equal to 250 is high; less than 250 is low), allergic and non-allergic, Th2 hi and low, Periostin hi and low (compared to median value), and FeNO hi and low (greater than or equal to 24 or less than 24).

The treatment also improves one or more symptoms of asthma as measured by an asthma symptom diary. Symptoms include, but are not limited to, daytime and nighttime symptom frequency and severity, activity avoidance and limitation, asthma-related stress and fatigue as well as rescue asthma medication use), and other measures of asthma control as measured by the Asthma Control Questionnaire omitting FEV₁ (ACQ-6).

In various embodiments, treatment with the anti-TSLP antibody delays the time to an asthma exacerbation compared to a subject not receiving the anti-TSLP antibody.

Also contemplated in the present disclosure is the administration of multiple agents, such as an antibody composition in conjunction with a second agent as described herein, including but not limited to an anti-inflammatory agent or asthma therapy.

However, it is contemplated that, in various embodiments, the administration reduces frequency of or levels of co-administered therapy in the subject. Exemplary co-administered therapies include, but are not limited to, inhaled corticosteroids (ICS), long-acting β2 agonist (LABA), leukotriene receptor antagonists [LTRA], long-acting anti-muscarinics [LAMA], cromones, short-acting β2 agonist (SABA), and theophylline or oral corticosteroids. In various embodiments, the administration eliminates the need for corticosteroid therapy.

### Formulations

In some embodiments, the disclosure contemplates use of pharmaceutical compositions comprising a therapeutically effective amount of an anti-TSLP antibody or antibody variant together with a pharmaceutically acceptable diluent, carrier, solubilizer, emulsifier, preservative, and/or adjuvant. In addition, the disclosure provides methods of treating a subject by administering such pharmaceutical composition.

In certain embodiments, acceptable formulation materials preferably are nontoxic to recipients at the dosages and concentrations employed. In certain embodiments, the pharmaceutical composition may contain formulation materials for modifying, maintaining or preserving, for example, the pH, osmolality, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18” Edition, (A. R. Genrmo, ed.), 1990, Mack Publishing Company.

A suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor.

The formulation components are present preferably in concentrations that are acceptable to the site of administration. In certain embodiments, buffers are used to maintain the composition at physiological pH or at a slightly lower pH, typically within a pH range of from about 5 to about 8. Including about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, and about 8.0.

In various embodiments, the anti-TSLP antibody or antibody variant is in a formulation containing sodium acetate, and one or more of proline, sucrose, polysorbate 20 or polysorbate 80. In various embodiments, the formulation comprises 1-50 mM sodium acetate, 3-9% (w/v) sucrose, 0.015% (w/v)±0.005% (w/v) polysorbate 20 or polysorbate 80, at pH between 4.9 and 6.0. Optionally, the antibody or antibody fragment is at a concentration of 70 mg/ml. The formulation may be stored at −20° to −70° C.

When parenteral administration is contemplated, the therapeutic compositions for use may be provided in the form of a pyrogen-free, parenterally acceptable aqueous solution comprising the desired anti-TSLP antibody in a pharmaceutically acceptable vehicle. A particularly suitable vehicle for parenteral injection is sterile distilled water in which the antibody is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the antibody.

### EXAMPLES

The present anti-TSLP antibody is the first epithelium-targeting product with potential for disruptive efficacy in patients with both non-eosinophilic and eosinophilic asthma. EOS-high populations make up approximately 50-70% of severe asthma patients.

The present example describes a multicenter, placebo-controlled, parallel-group, double-blind phase 2 study conducted at 108 study sites across 12 countries. Eligible patients were current non-smokers (for ≥6 months and with a history of <10 pack-years) who were aged 18-75 years and who had asthma that was not well-controlled despite treatment LABAs combined with a medium dose (250 to 500 μg per day of fluticasone administered by means of a dry-powder inhaler or equivalent) or high dose (>500 μg per day of fluticasone administered by means of a dry-powder inhaler or equivalent) of inhaled glucocorticoids (as per GINA 2012 guidelines defining severe asthma²⁴) at least 6 months prior to enrollment. Patients were also required to have a history of at least two asthma exacerbations that led to systemic glucocorticoid treatment, or one severe exacerbation that led to hospitalization, in the 12 months before trial entry. Additional eligibility criteria included pre-bronchodilator (forced expiratory volume in 1 second (FEV₁) of at least 40% and no more than 80% of predicted, post-bronchodilator reversibility of at least 12% and at least 200 ml, and a score on the six-item Asthma Control Questionnaire (ACQ-6)25 score at least 1.5 during screening (range, 0 to 6, with lower scores indicating better disease control; minimal clinically important difference, 0.5).²⁶ Exclusion criteria included any clinically important pulmonary disease other than asthma. A full list of the inclusion and exclusion criteria is provided in Table 1A.

Patients were randomly assigned (in a 1:1:1:1 ratio), according to a central interactive voice-response or Web-response system, to receive one of three different doses of subcutaneous (SC) tezepelumab, a bivalent antibody having identical binding sites to TSLP, or placebo. Randomization was stratified by location (Japan or rest of world), blood eosinophil count (≥250 cells/μl or <250 cells/μl) as measured by a local laboratory, and dose level of inhaled glucocorticoids (medium or high, on the basis of GINA 2012 guidelines).²⁶ Patients receiving a maintenance regimen of oral glucocorticoids were assigned to the high-dose inhaled glucocorticoid stratum. Tezepelumab and placebo were prepared by site staff who were aware of the trial-group assignments and were not involved in trial assessments. The trial agents were similar in appearance and administered by staff who were unaware of the trial-group assignments. Background asthma control medications were maintained at a stable dose throughout the treatment period.

### Procedures

Patients were assigned to receive SC injections of tezepelumab 70 mg every 4 weeks (Q4W, low dose), 210 mg Q4W (medium dose), or 280 mg every 2 weeks (Q2W, high dose), or placebo Q2W for the duration of the trial. To maintain blinding, patients who were assigned to randomized to the Q4W dosing regimens received placebo at the intermediate visits.

Baseline measurements of pre-bronchodilator and post-brochodilator spirometric assessments of fractional exhaled nitric oxide (Fe_{NO}), blood eosinophil counts, ACQ-6 score, and the score on the asthma quality of life questionnaire (standardized) for persons 12 tears of age or older (AQLQ[S]+12 [hereafter referred to as AQLQ])27 were obtained throughout the 5-week screening period. The ACQ-6 score, AQLQ score, and Asthma symptom score (reflecting daytime severity, daytime frequency, and nighttime severity; range 0 [no symptoms] to 4 [worst possible symptoms]) were recorded using an electronic device. Safety was monitored at each study site from enrollment through follow-up at week 64.

### Endpoints and Assessments

The primary efficacy endpoint was the annualized asthma exacerbation rate (AER) at week 52. An asthma exacerbation was defined as a worsening of asthma symptoms that led to any of the following: 1) use of systemic glucocorticoids (oral or injectable) or, in the case of stable maintenance regimen of oral glucocorticoids, a doubling of the dose for three or more days; 2) an emergency department visit due to asthma that led to systemic glucocorticoid treatment; or 3) an inpatient hospitalization due to asthma. Worsening of asthma was defined as new or increased symptoms or signs that were either worrisome to the patient or related to an asthma diary driven alert.

Secondary endpoints included change from baseline in prebronchdilator and postbronchodilator FEV1 (an increase in values indicates improved lung function; minimal clinically important difference, 100 to 200 ml), ACQ-6 score, AQLQ score, asthma symptom score, forced vital capacity (FVC), as well as the annualized rate of severe asthma exacerbations at week 52; the time to the first asthma exacerbation, the time to the first severe asthma exacerbation; the percentage of patients with at least one asthma exacerbation, and the percentage of patients with at least one severe asthma exacerbation.

Primary and secondary end points (changes from baseline in prebronchodilator FEV1, ACQ-6 score, AQLQ score, and asthma symptom score) were also assessed in pre-specified subpopulations according to blood eosinophil count (≥250 or <250 cells per microliter), Th2 status (high [IgE level >100 IU per milliliter and blood eosinophil count ≥140 cells per microliter] or low [IgE level ≤100 IU per milliliter or blood eosinophil count <140 cells per microliter]),³⁰ FENO level (on the basis of median baseline levels and the clinically meaningful cutoff of 24 ppb),³¹ serum periostin level (high or low, on the basis of median baseline levels), current (demonstrated during the screening period) postbronchodilator FEV1 reversibility, and allergic status (defined by a positive or negative fluorescence enzyme immunoassay for IgE at baseline).

The primary end point was also stratified according to dose level of inhaled glucocorticoids (medium or high), use or nonuse of a maintenance regimen of oral glucocorticoids, and number of asthma exacerbations in the previous 12 months (pre-specified subgroup analyses). Post hoc analyses included stratification of the primary end point according to baseline blood eosinophil count (<400 or ≥400 cells per microliter) and patient smoking history.

### Statistical Analysis

The efficacy analysis was based on the intent-to-treat (ITT) population, which consisted of patients who underwent randomization and received at least one dose of tezepelumab or placebo and analyzed according to the randomized trial group. The safety analyses were based on the as-treated population and included all the patients who received at least one dose of tezepelumab or placebo; patients were evaluated according to trial agent received.

For the primary efficacy endpoint, 138 patients per trial group were required for 80% power to detect a 40% lower annualized rate of asthma exacerbations in each tezepelumab dose group than in the placebo group, with a two sided alpha level of 0.1 and an expected 10% loss of information due to dropouts, under the assumption of an annualized asthma exacerbation rate of 0.7 events in the placebo group and a negative binomial dispersion parameter of 0.7.

The primary efficacy endpoint of annualized rate of asthma exacerbations was analyzed using a negative binomial model, with trial group, baseline blood eosinophil count (≥250 or <250 cells/μl) and baseline dose level of glucocorticoids (medium or high) included in the model. Continuous secondary endpoints were analyzed using a mixed-effects model for repeated measures analysis. Time-to-first event variables were analyzed using a Cox proportional hazard model. The categorical variables were analyzed using a Pearson's chi-squared test.

The primary endpoint was tested sequentially to control overall type-I error rate at 0.1. The hierarchy was tezepelumab high dose tezepelumab (280 mg Q2W) versus placebo, medium dose tezepelu, mab (210 mg Q4W) versus placebo, and low dose tesepelumab (70 mg Q4W) versus placebo. No adjustments for multiplicity for the secondary endpoints was applied. Nominal P values are presented. All analyses were done using SAS version 9.3.

### Results

### Patients

Analysis A, primary analysis after database lock, all sites included: Overall, 918 subjects were screened and 584 patients underwent randomization: 145 were assigned to low dose tezepelumab (70 mg Q4W), 145 were assigned to medium dose tezepelumab (210 mg Q4W), 146 were assigned to high dose tezepelumab (280 mg Q2W) and 148 were assigned the placebo. Of the patients who received tezepelumab or placebo, and were included in the ITT population, 391 (89.7%) and 139 (93.9%) completed treatment, respectively. Baseline and clinical characteristics were similar across groups.

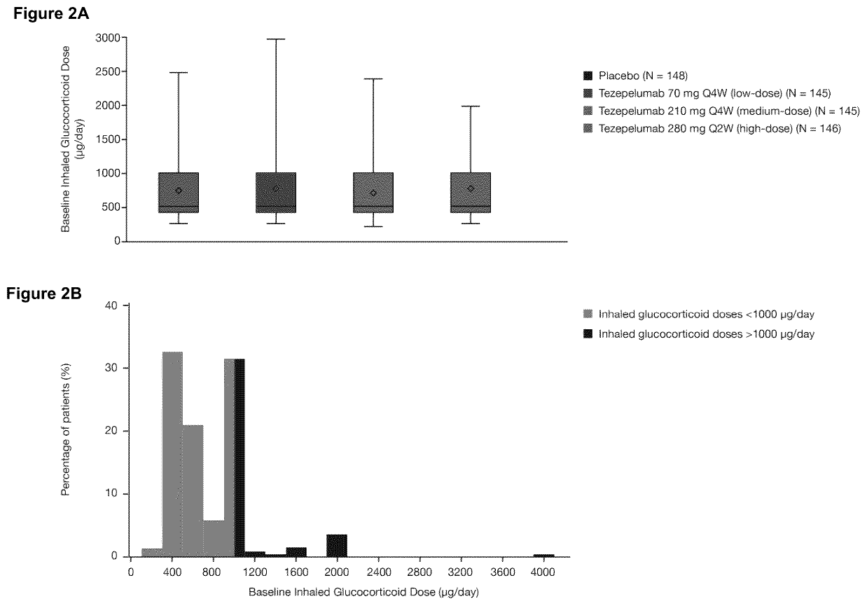

The dose range of inhaled glucocorticoids for patients at baseline is shown in FIGS. 2A and 2B. The median dose was 400 μg per day of fluticasone administered by means of a dry-powder inhaler or equivalent in the medium-dose inhaled glucocorticoid stratum, with 73 patients in the placebo group, 71 in the low-dose tezepelumab group, 70 in the medium-dose group, and 72 in the high-dose group, and 1000 μg per day of fluticasone administered by means of a dry-powder inhaler or equivalent in the high-dose inhaled glucocorticoid stratum, with 75, 74, 75, and 74 patients in the respective trial groups.

### Primary Endpoint

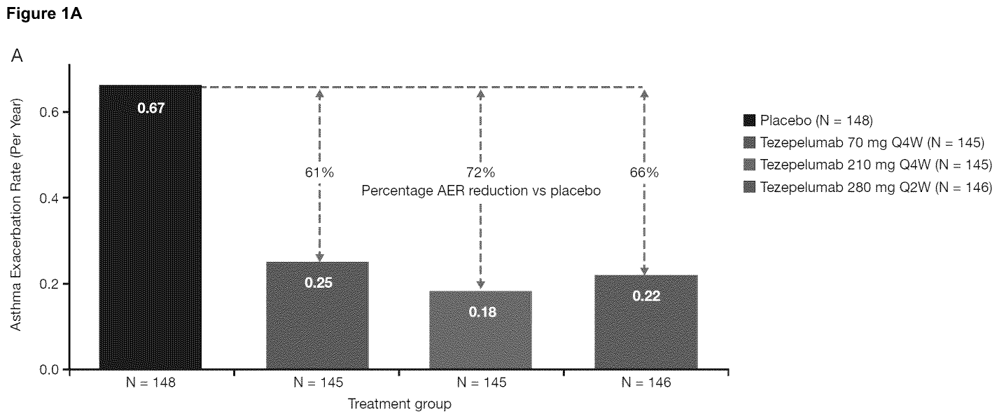

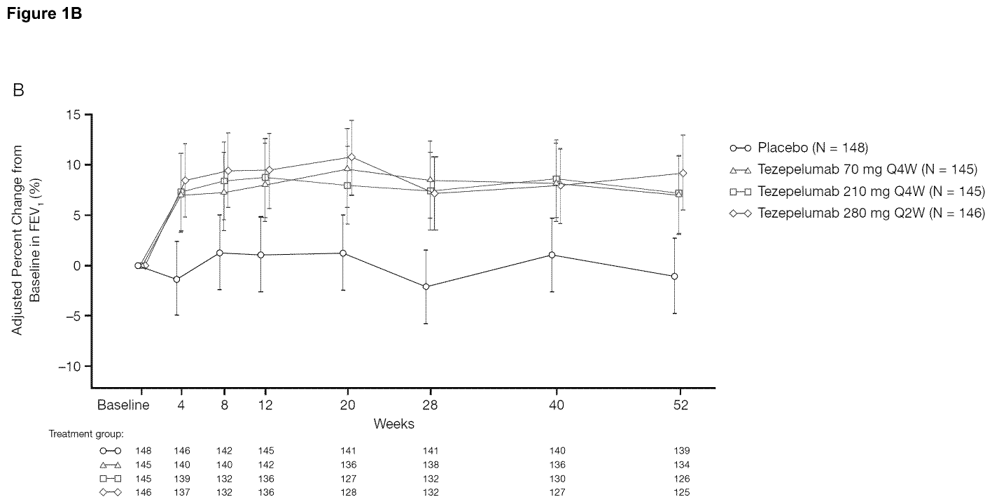

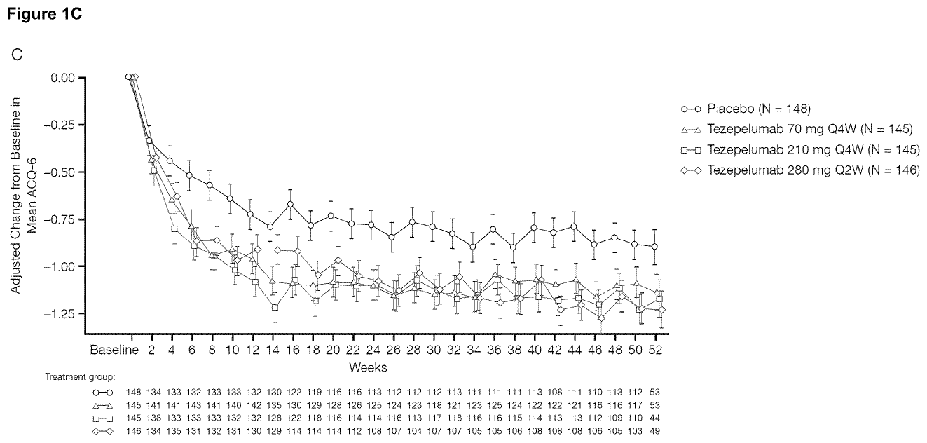

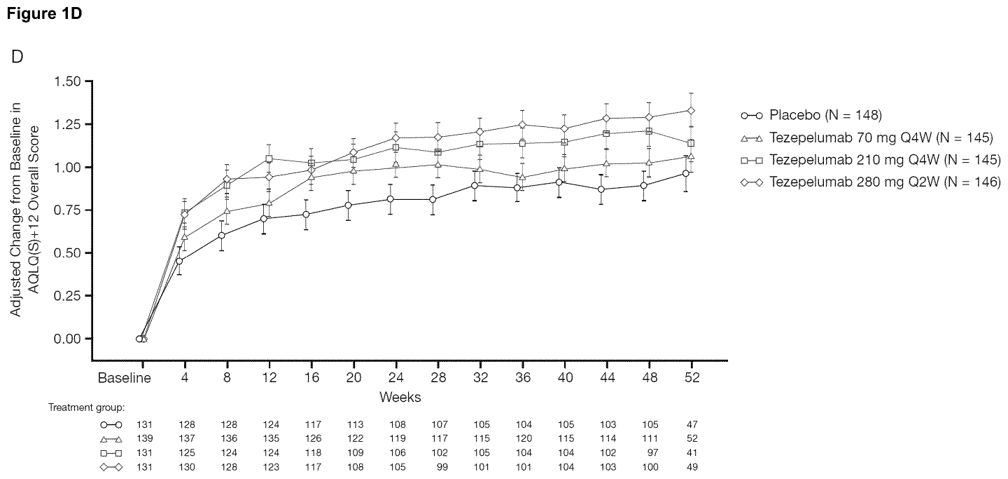

Treatment with tezepelumab resulted in annualized rates of asthma exacerbations at week 52 of 0.25, 0.18, and 0.22 events in the low-dose, medium-dose, and high-dose groups, respectively, as compared with 0.67 events in the placebo group. Thus, exacerbation rates were lower in the tezepelumab groups than in the placebo group by 61% (90% confidence interval [Cl], 39 to 75; P<0.001), 72% (90% Cl, 54 to 83; P<0.001), and 66% (90% Cl, 46 to 79; P<0.001), respectively (Table 2, and FIG. 1A). The types of asthma exacerbations that were used for the primary analysis are described in Table 1B.

### Secondary Endpoints

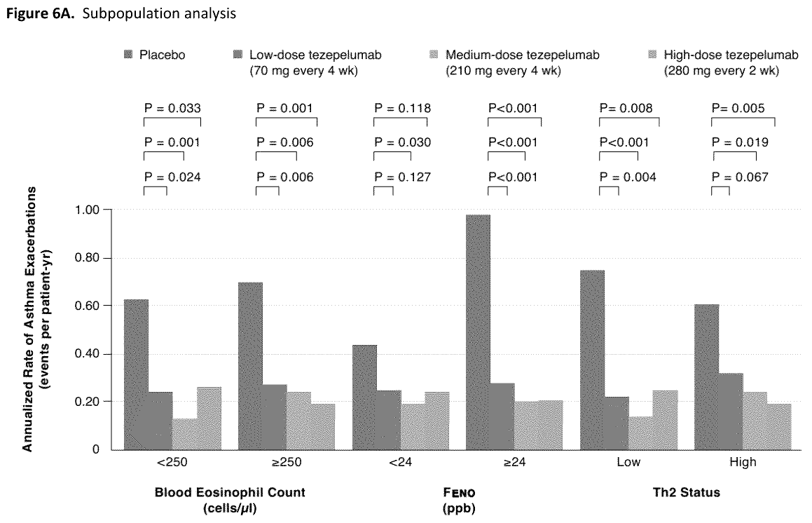

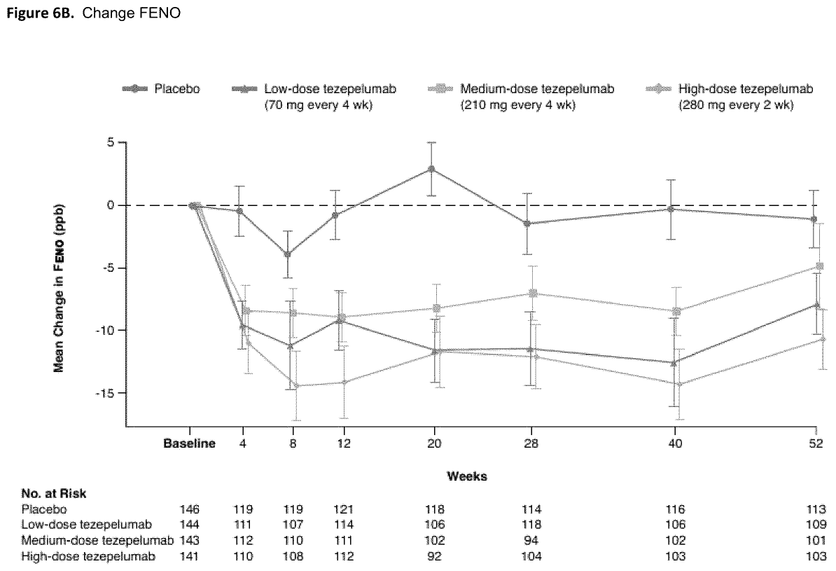

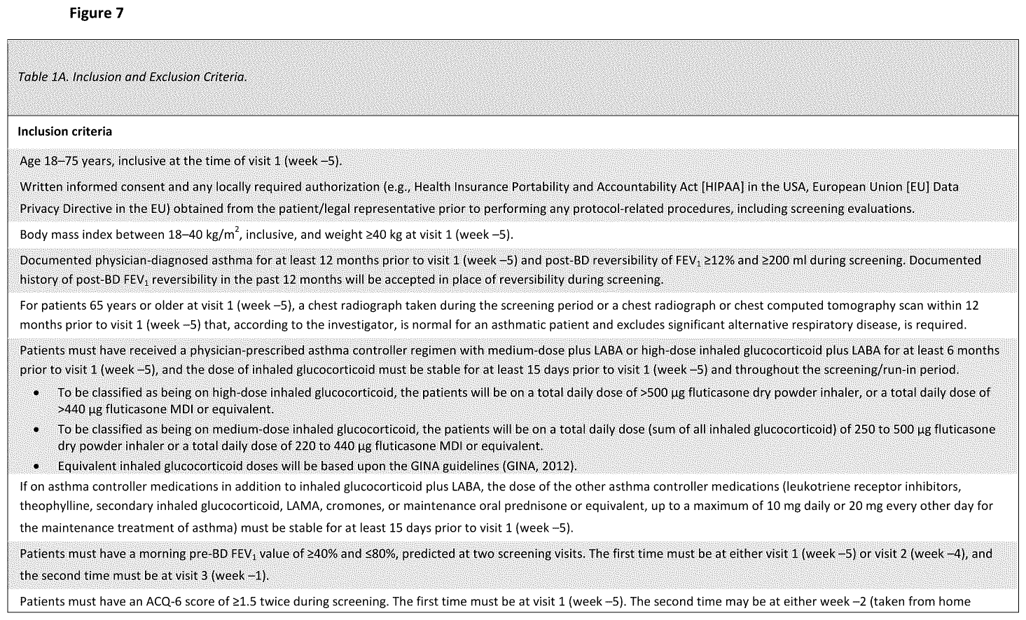

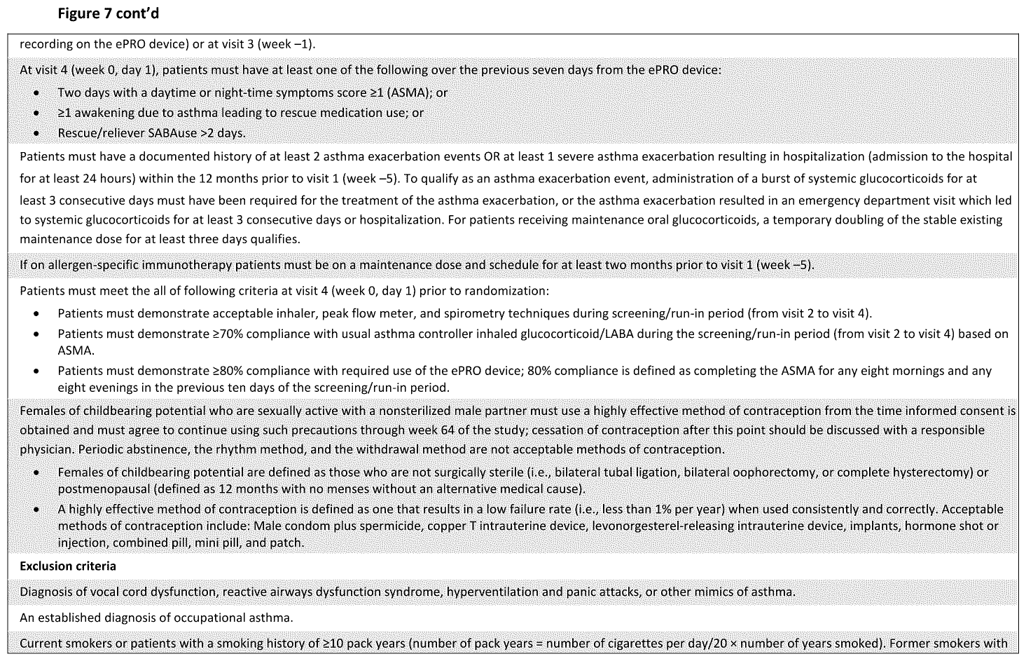

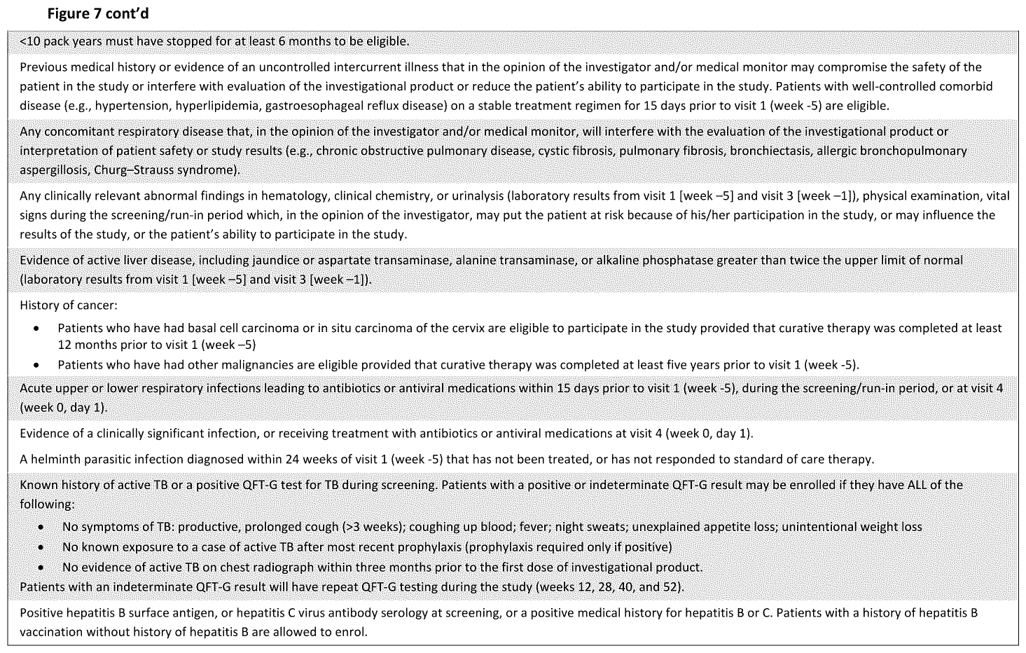

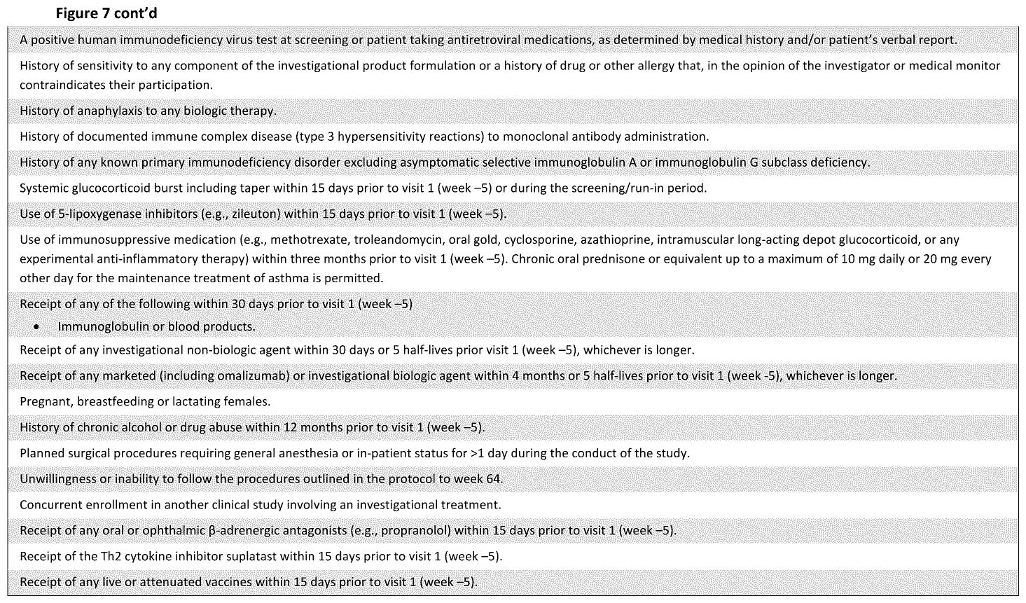

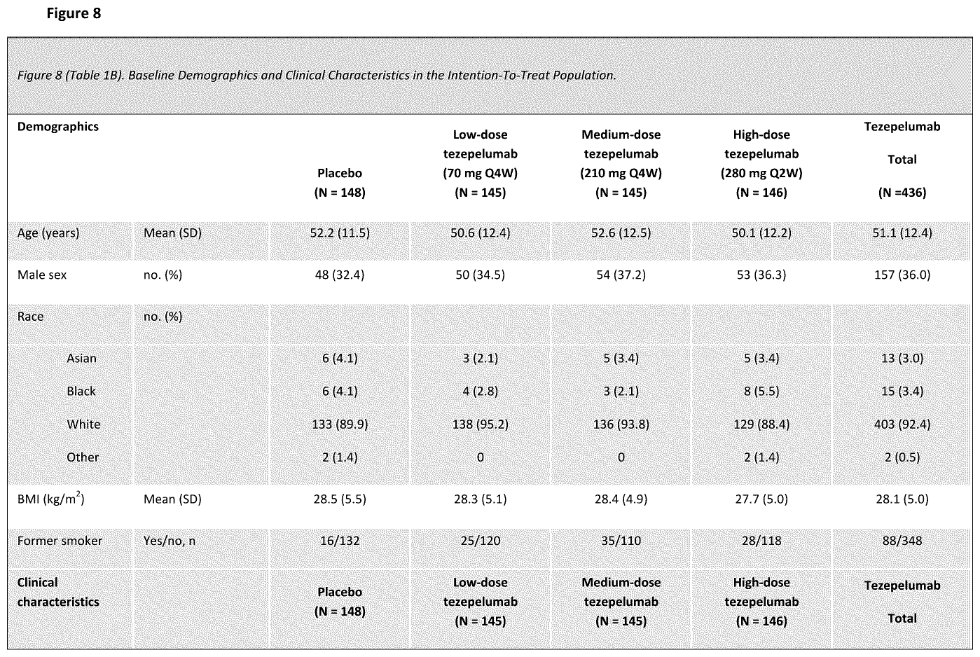

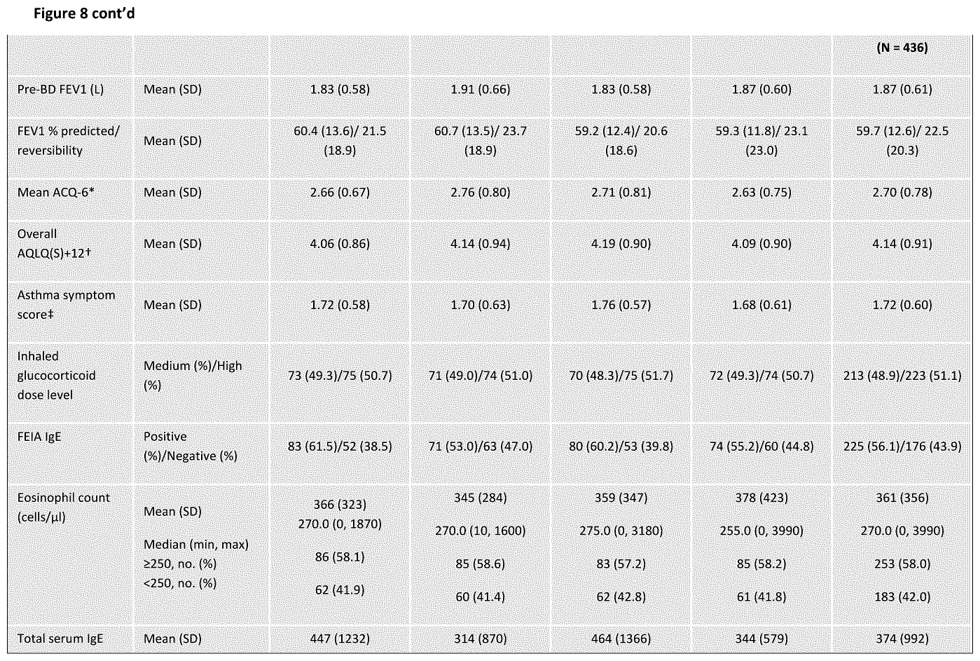

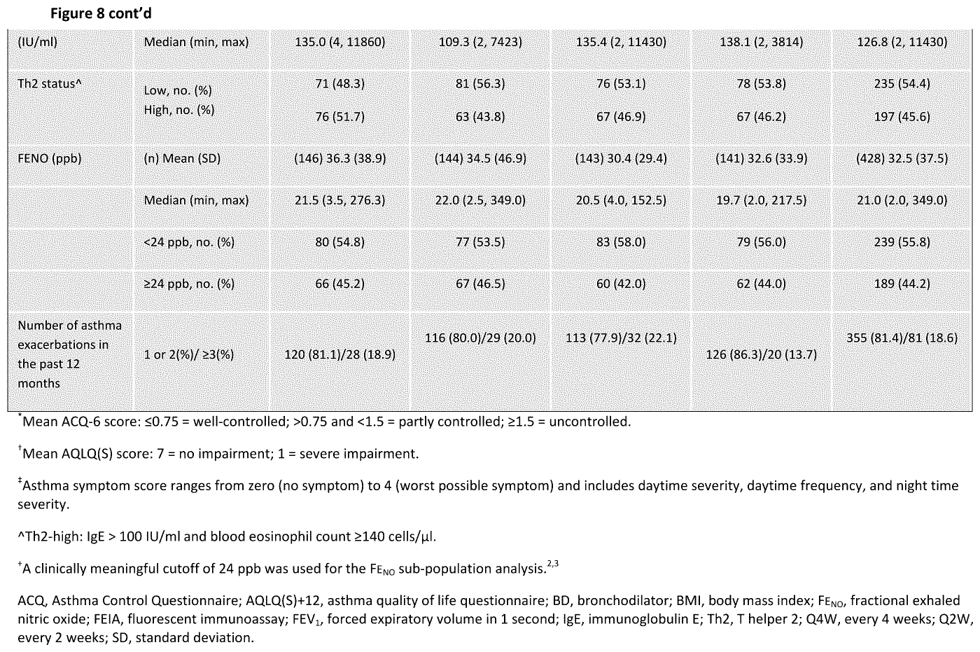

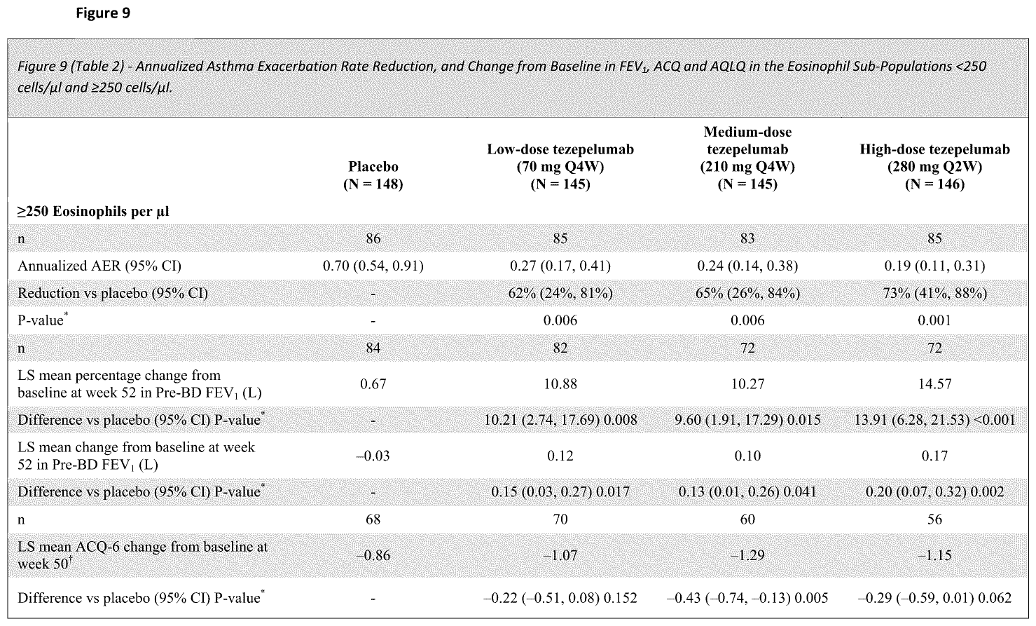

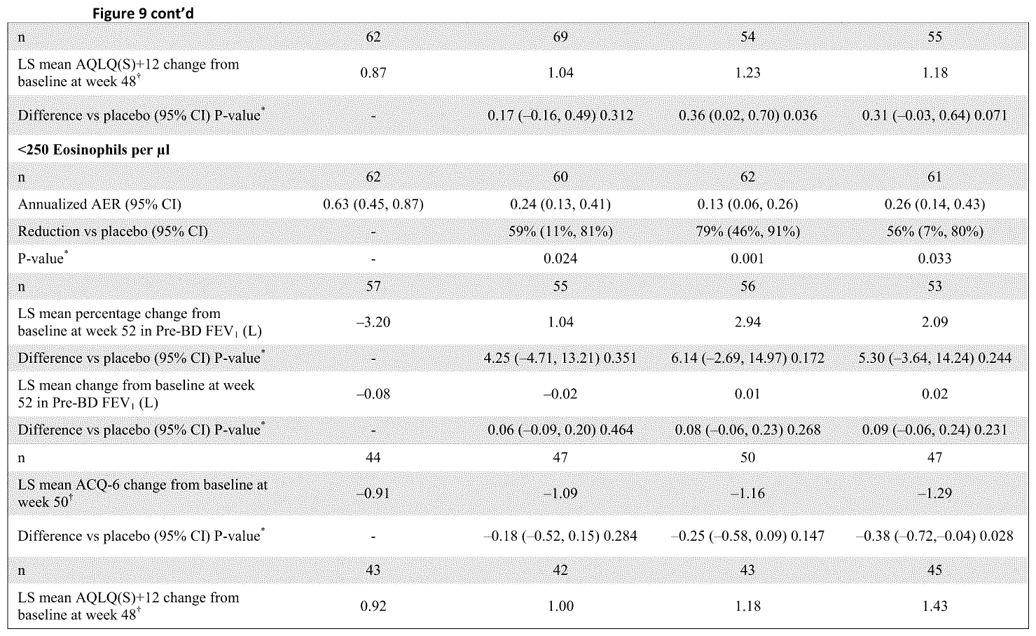

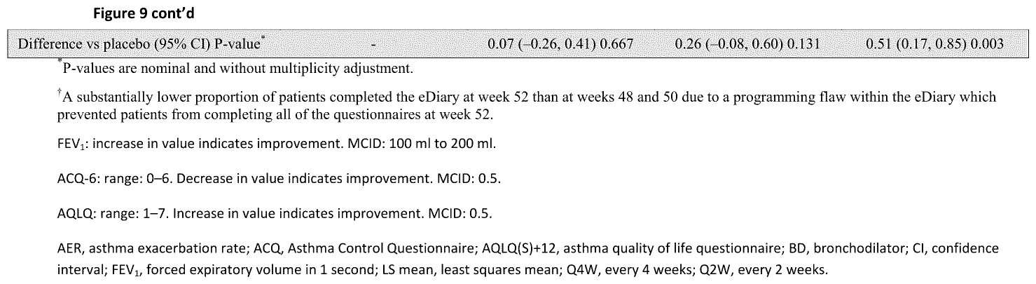

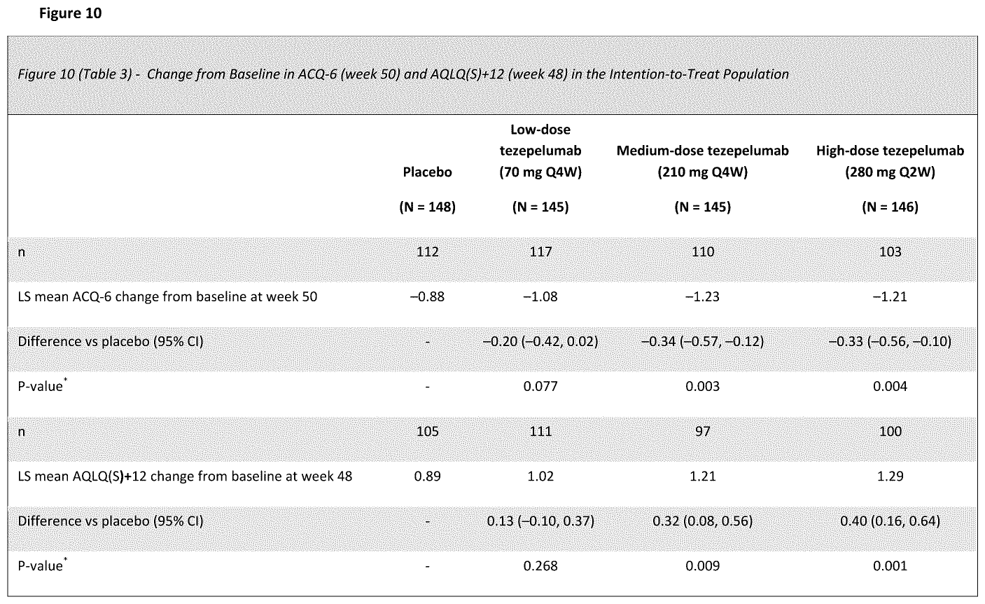

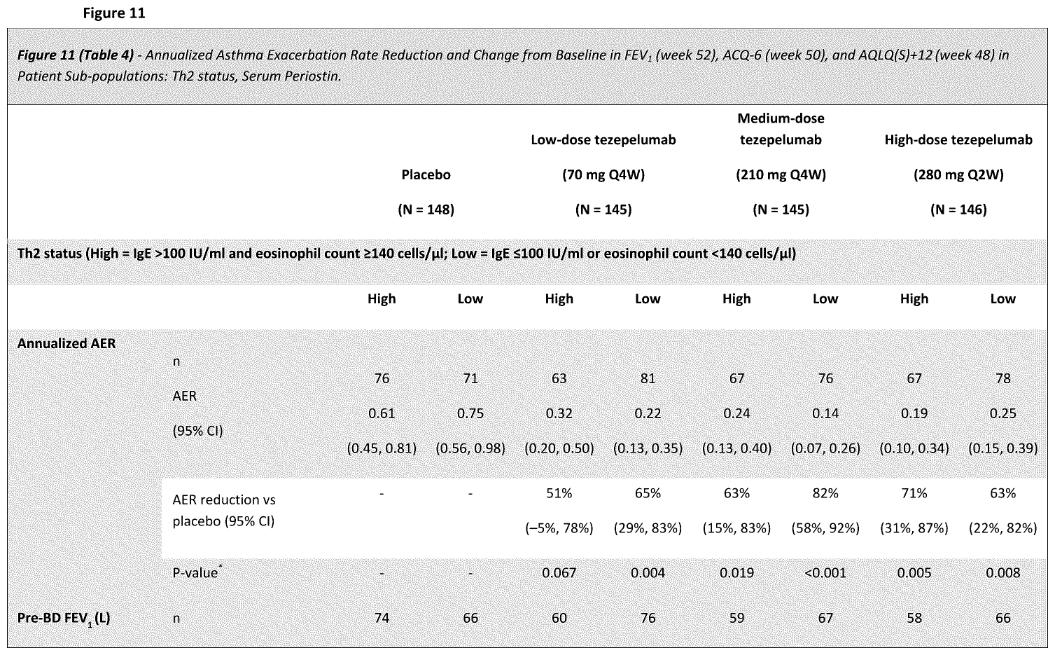

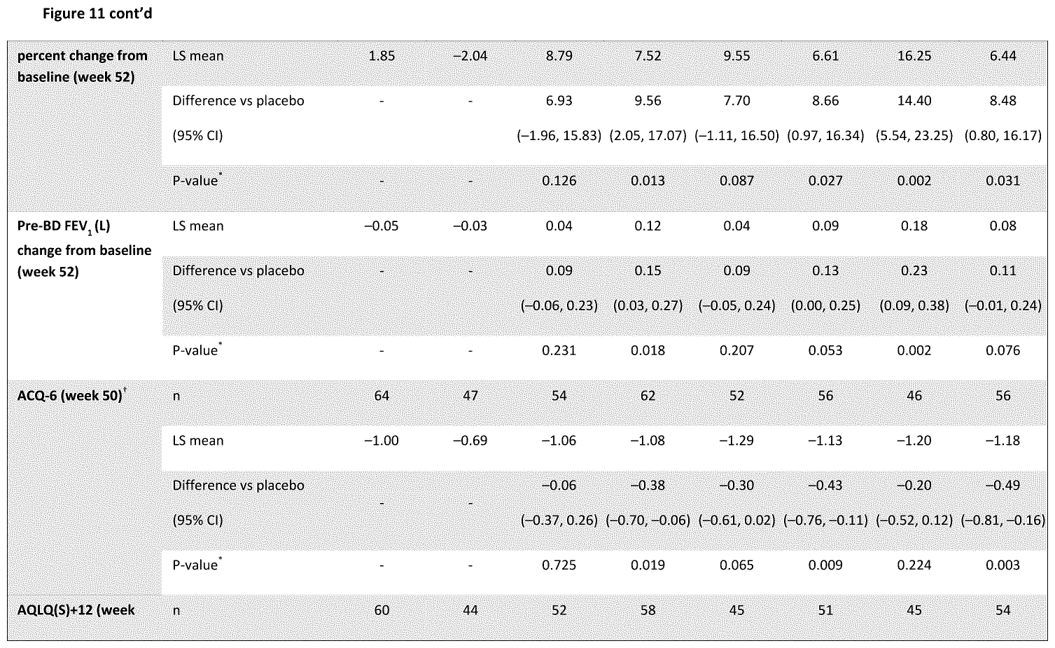

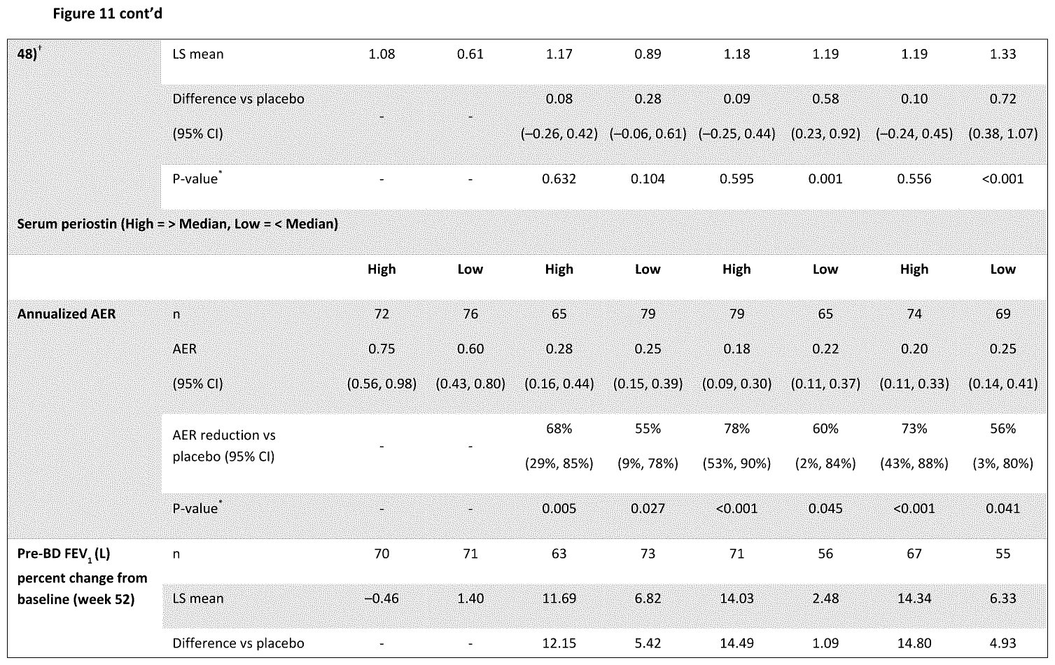

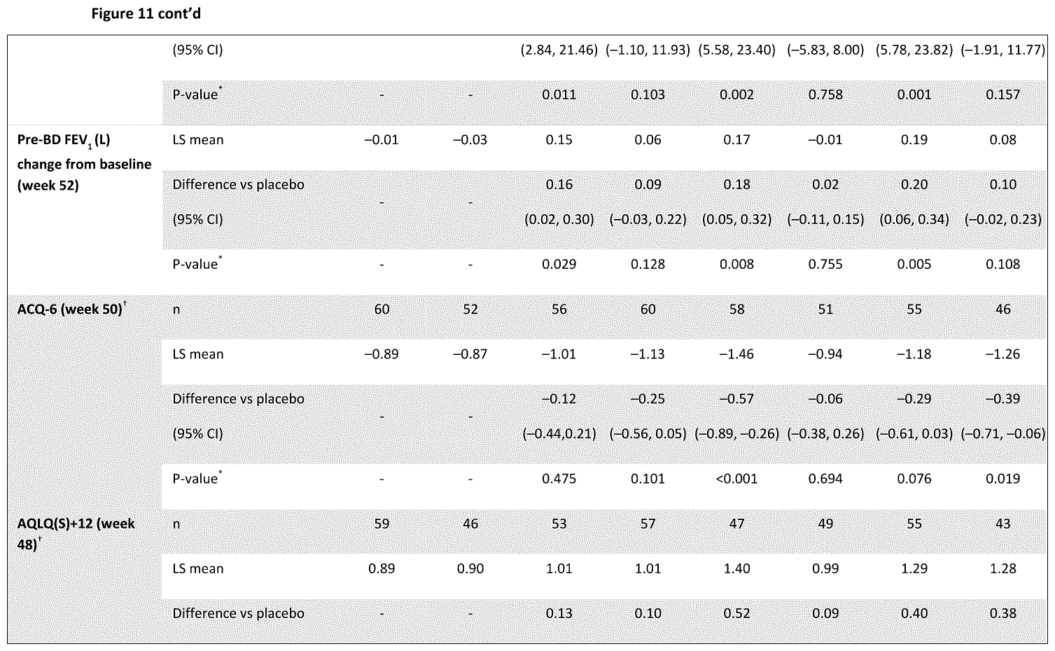

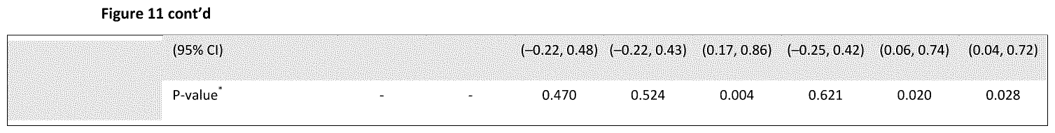

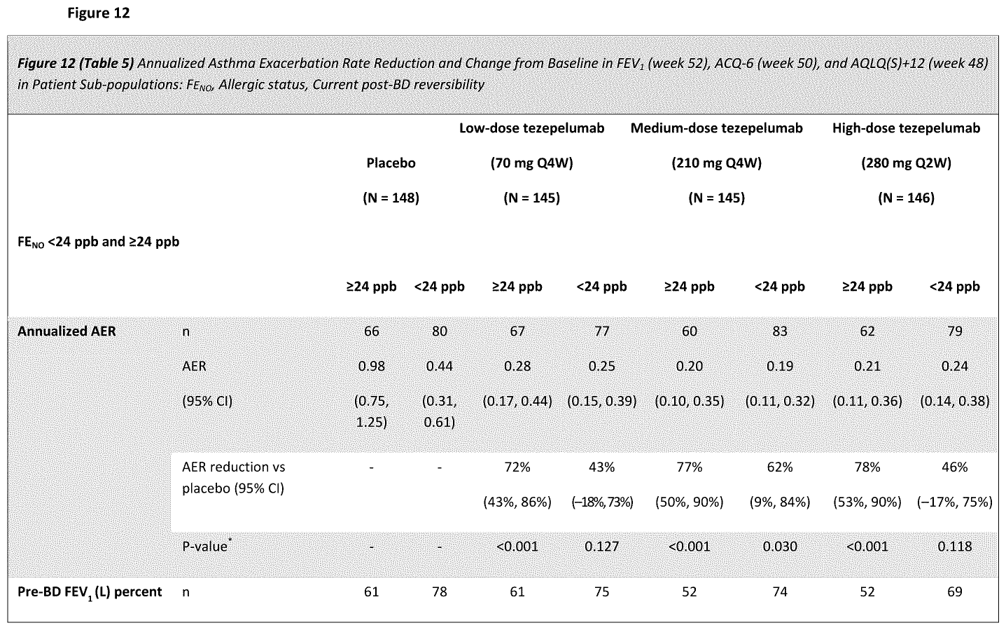

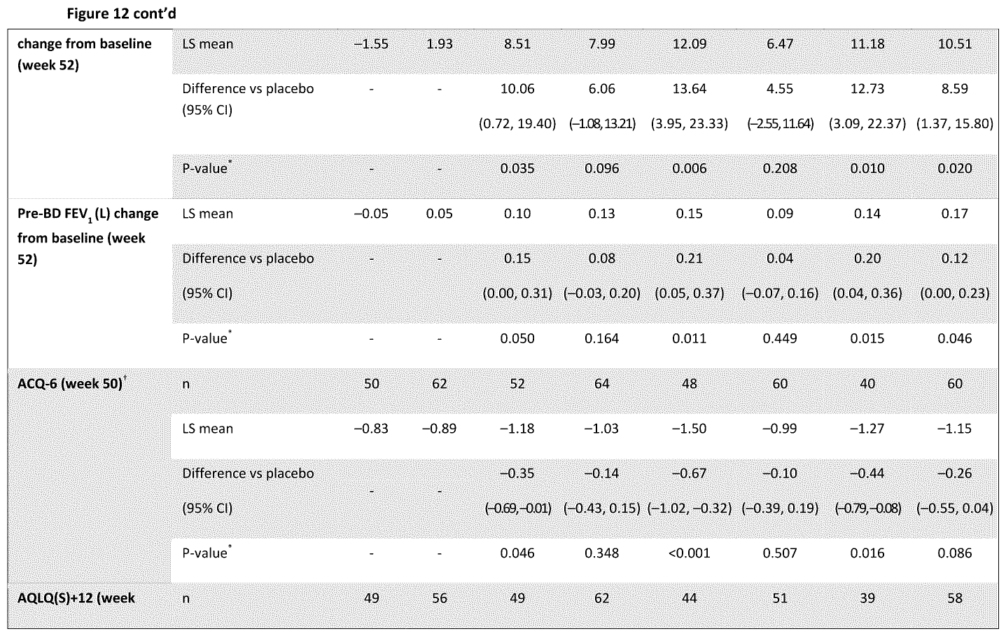

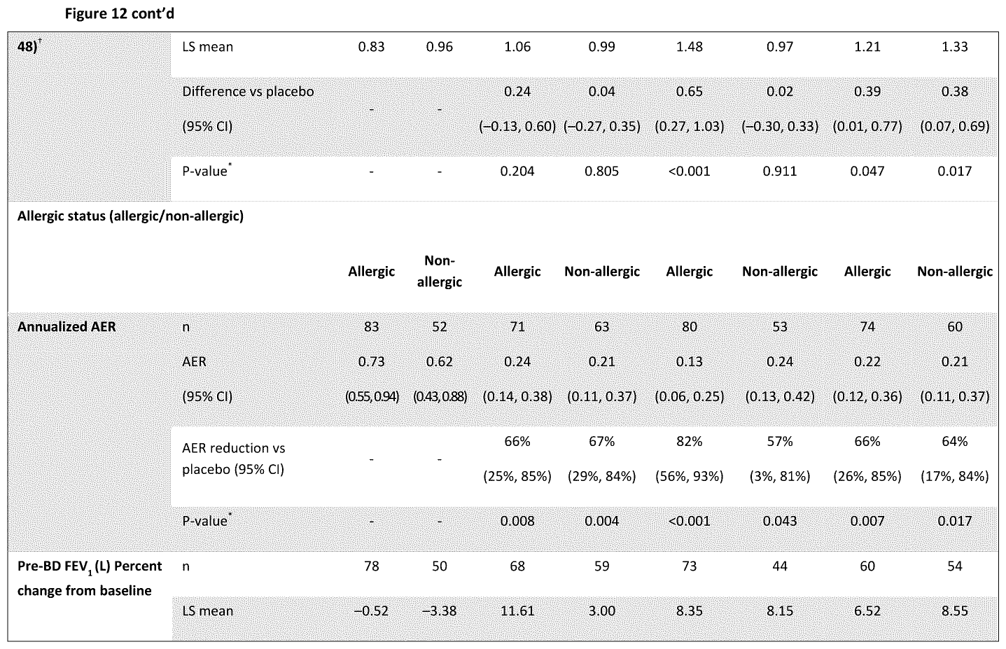

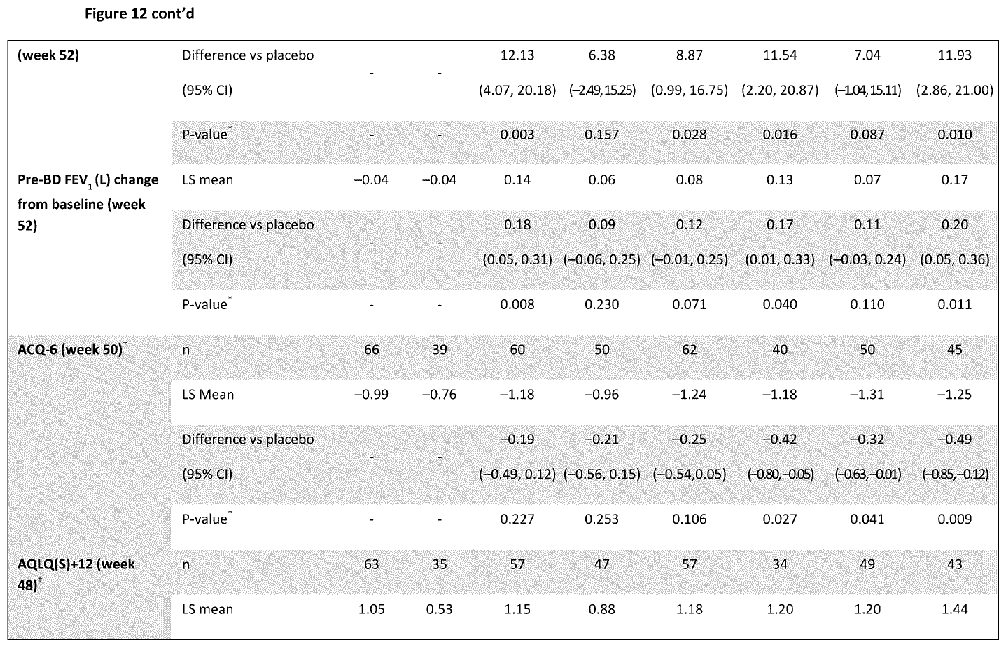

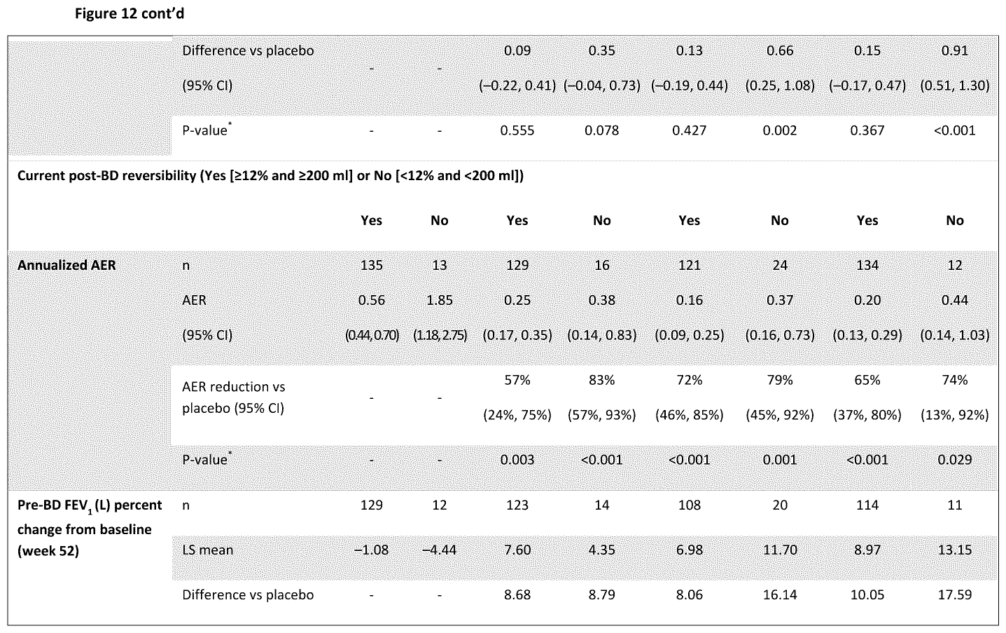

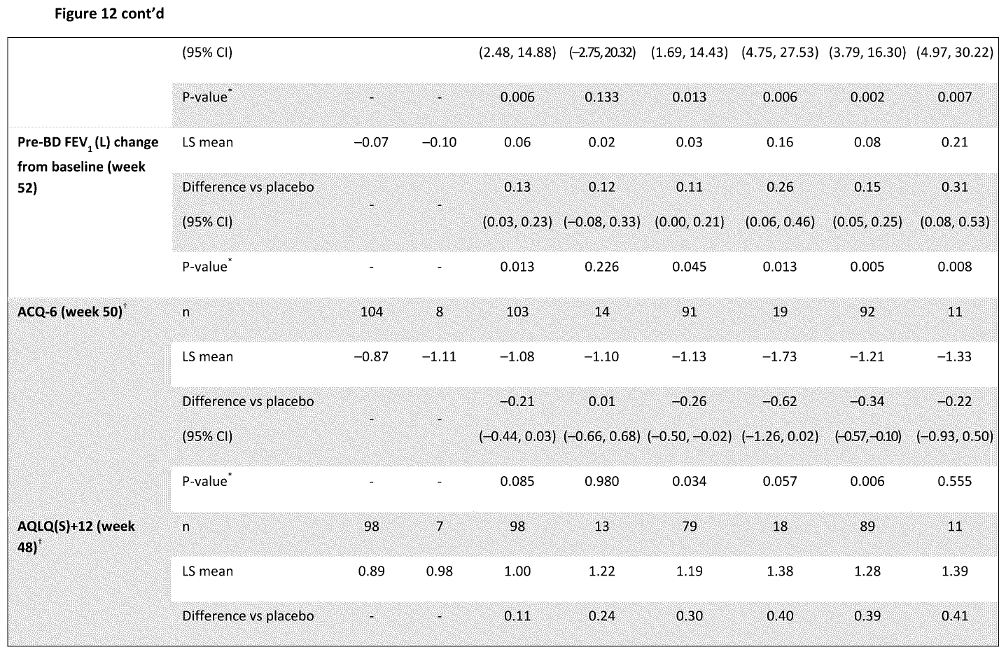

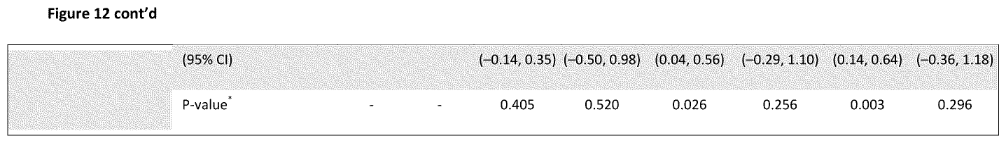

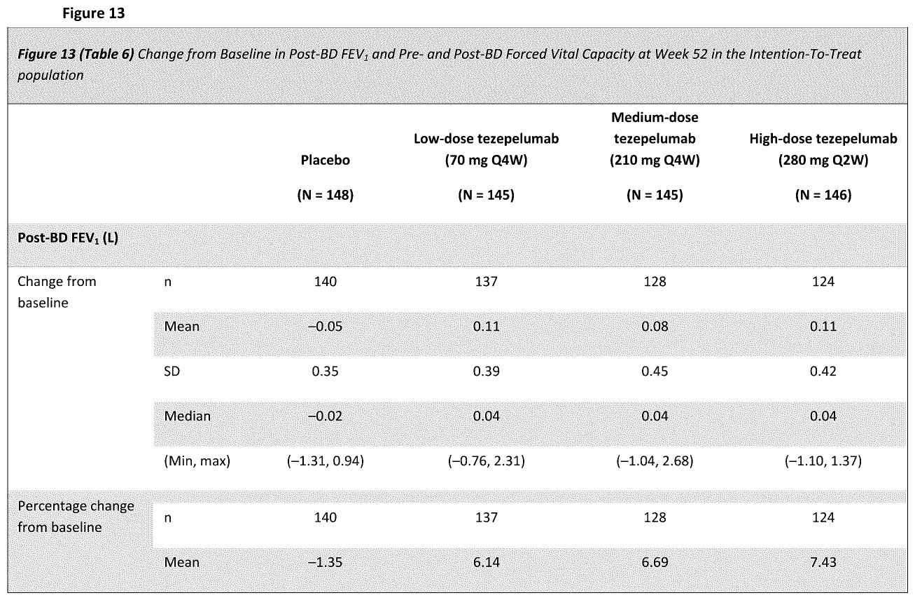

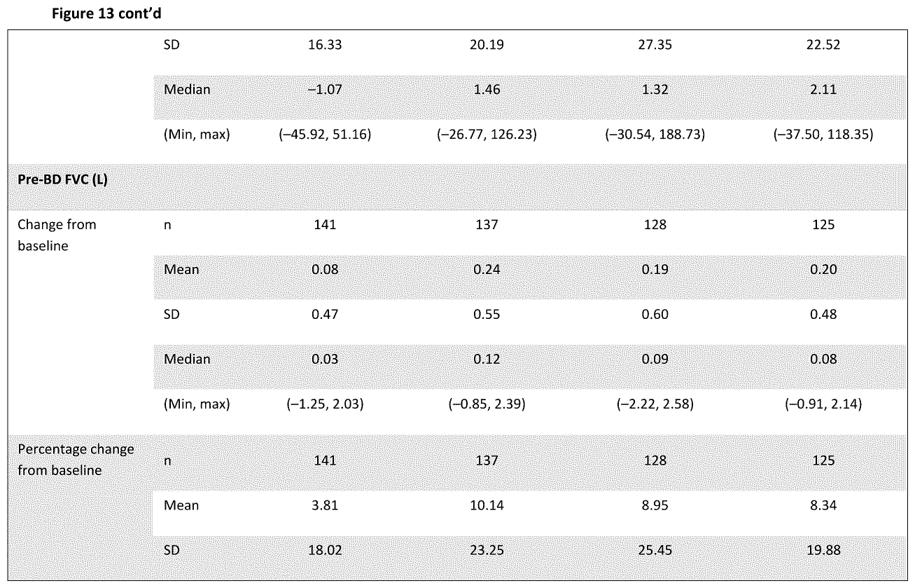

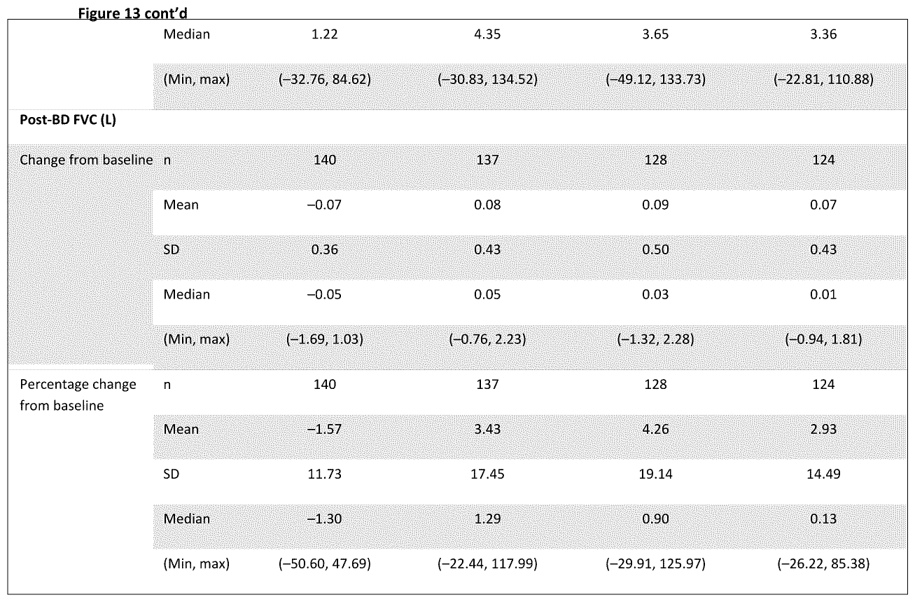

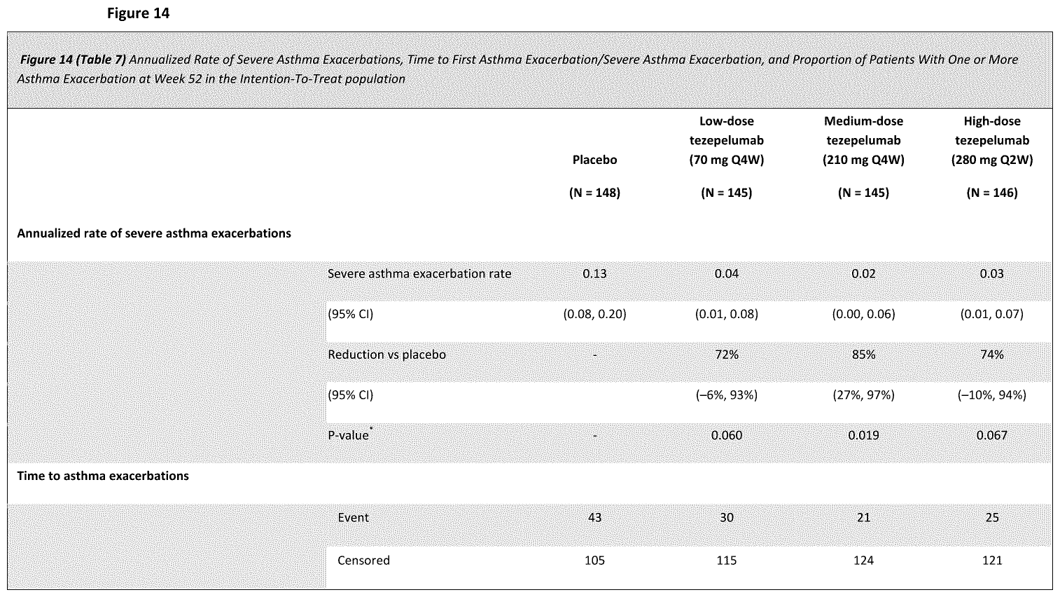

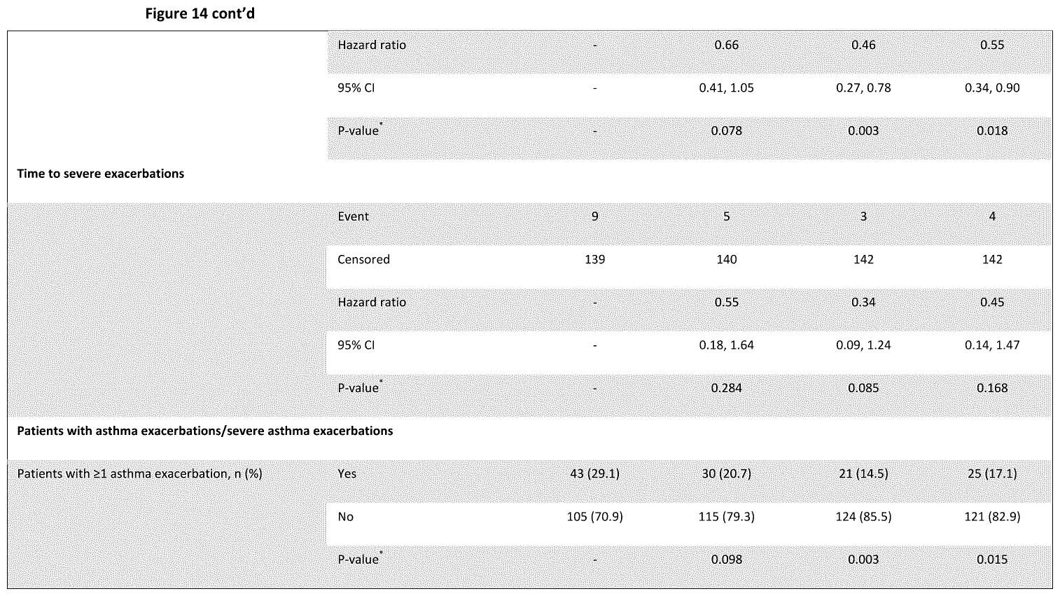

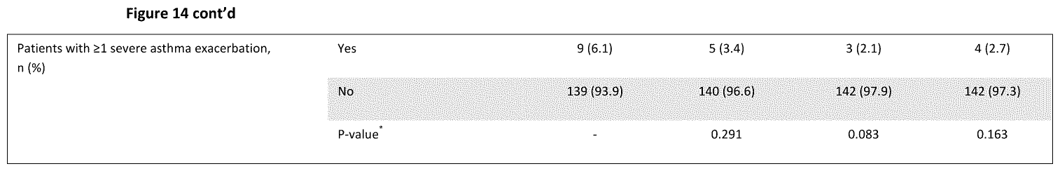

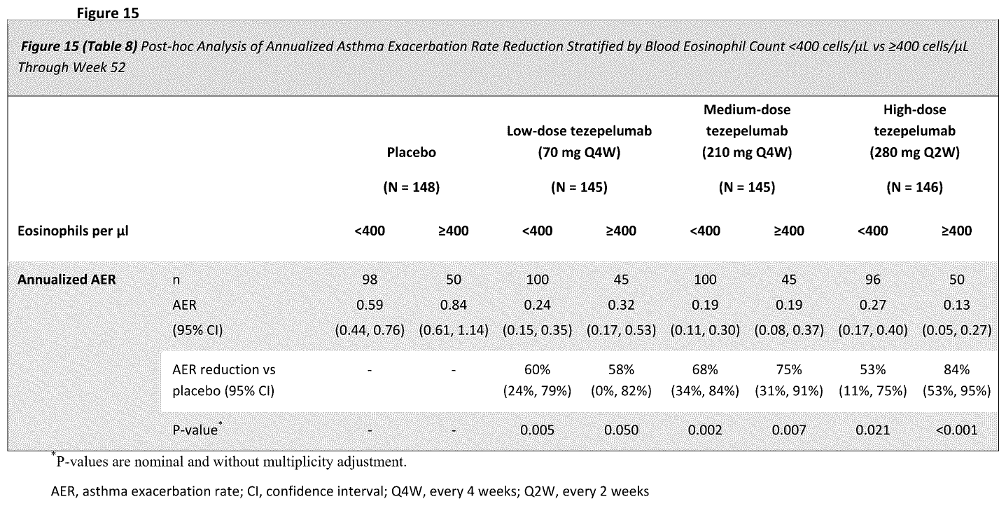

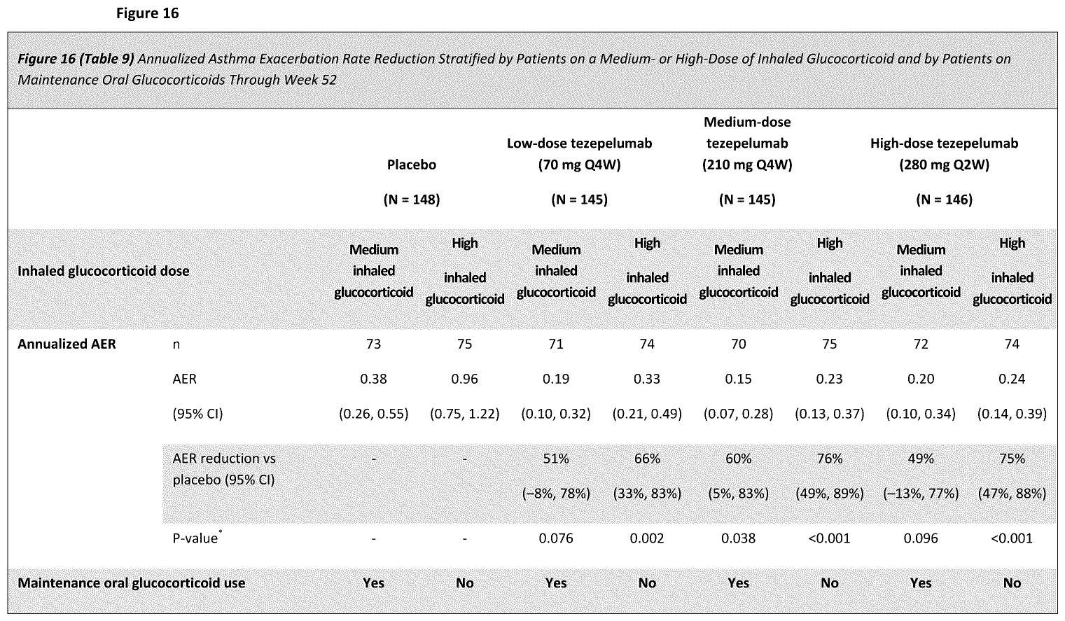

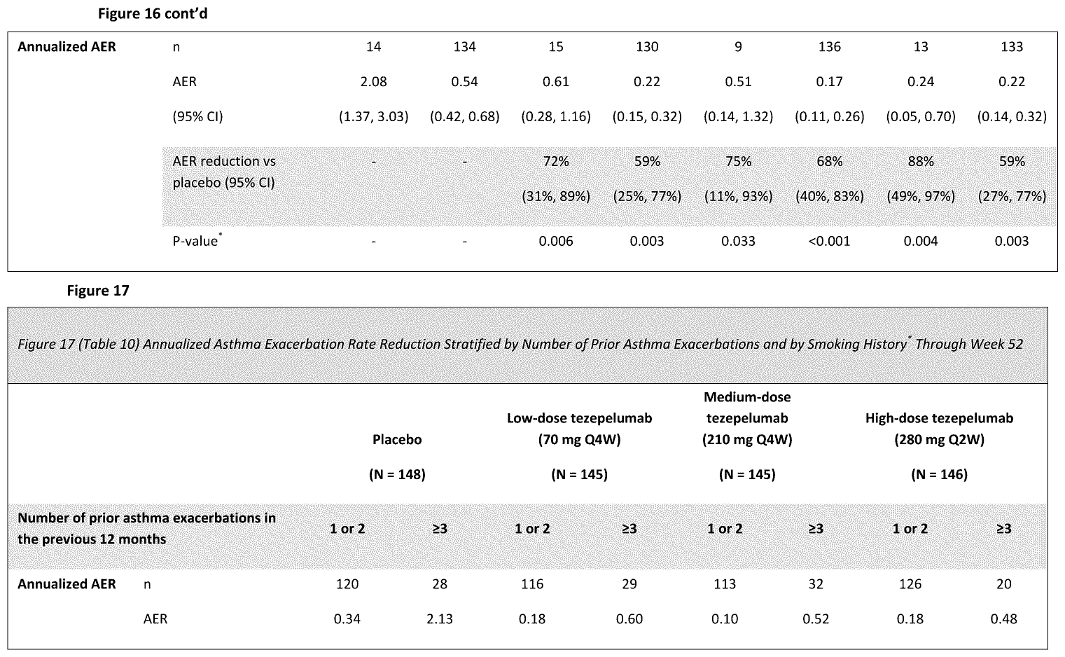

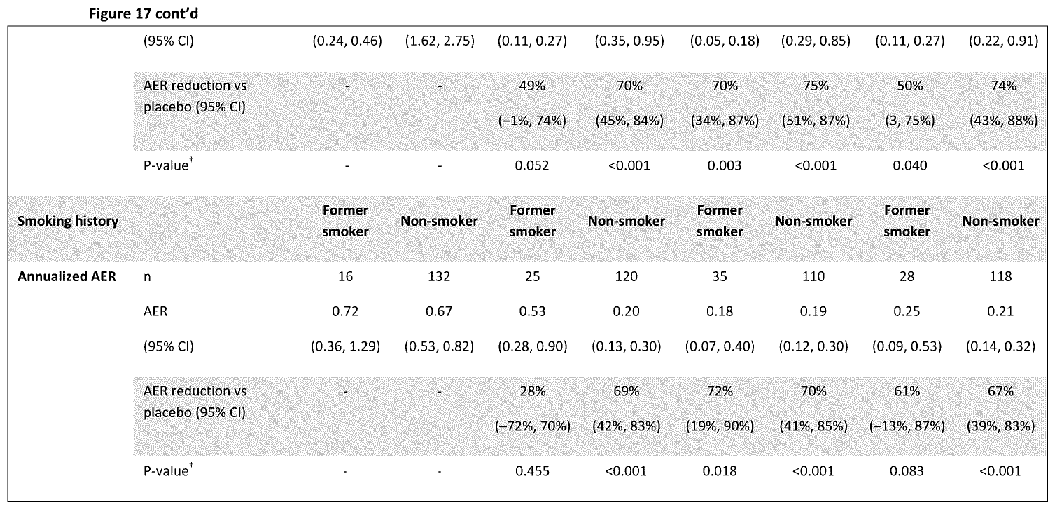

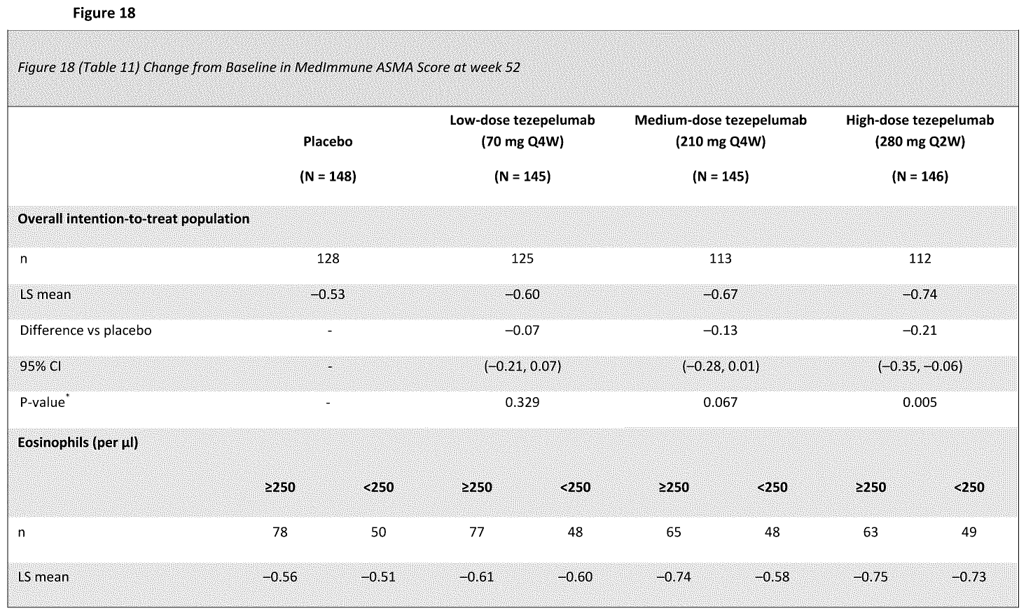

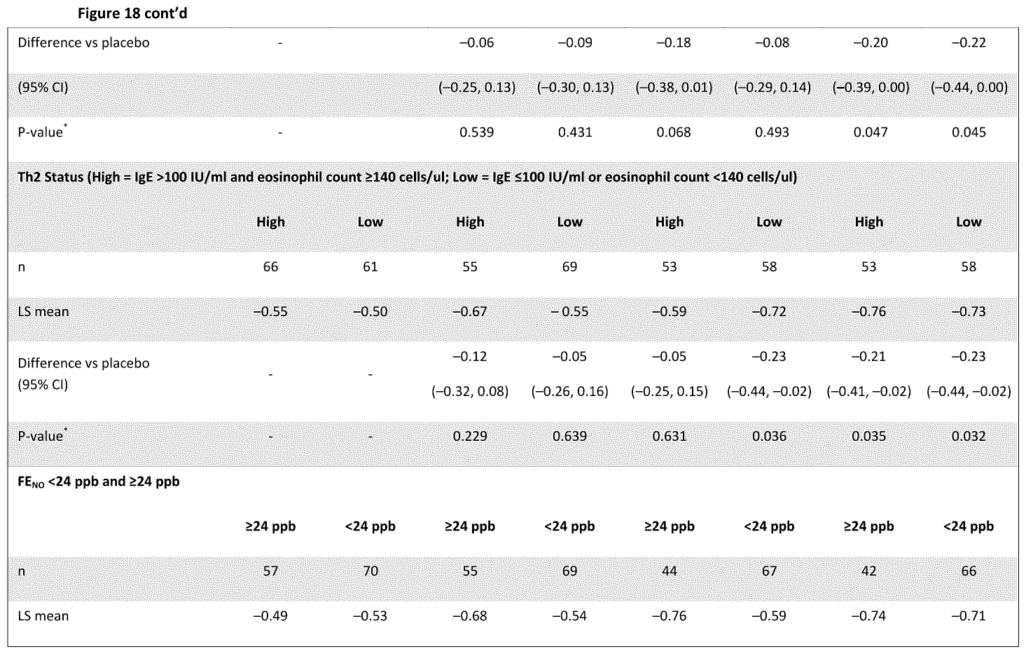

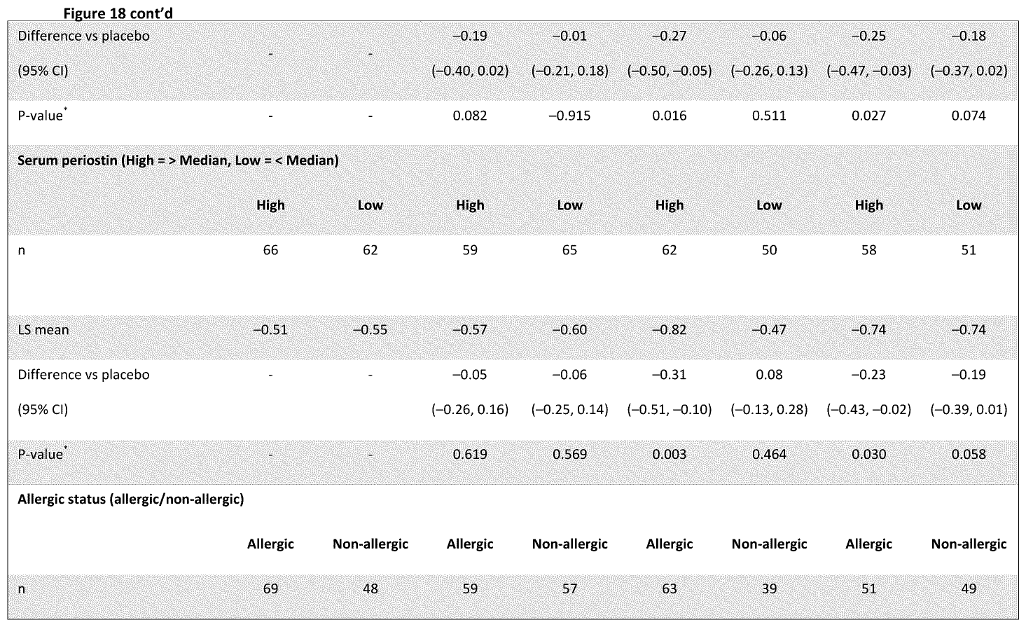

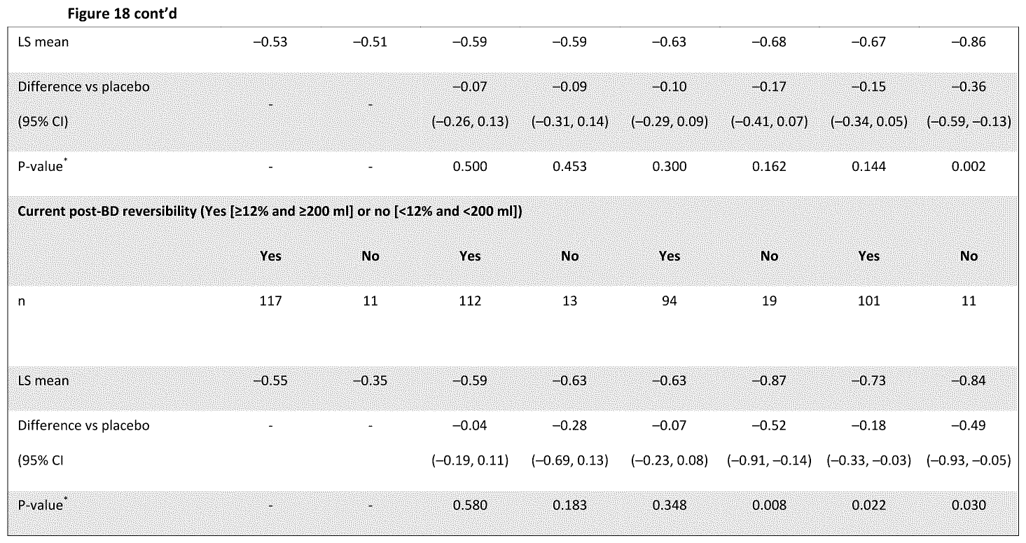

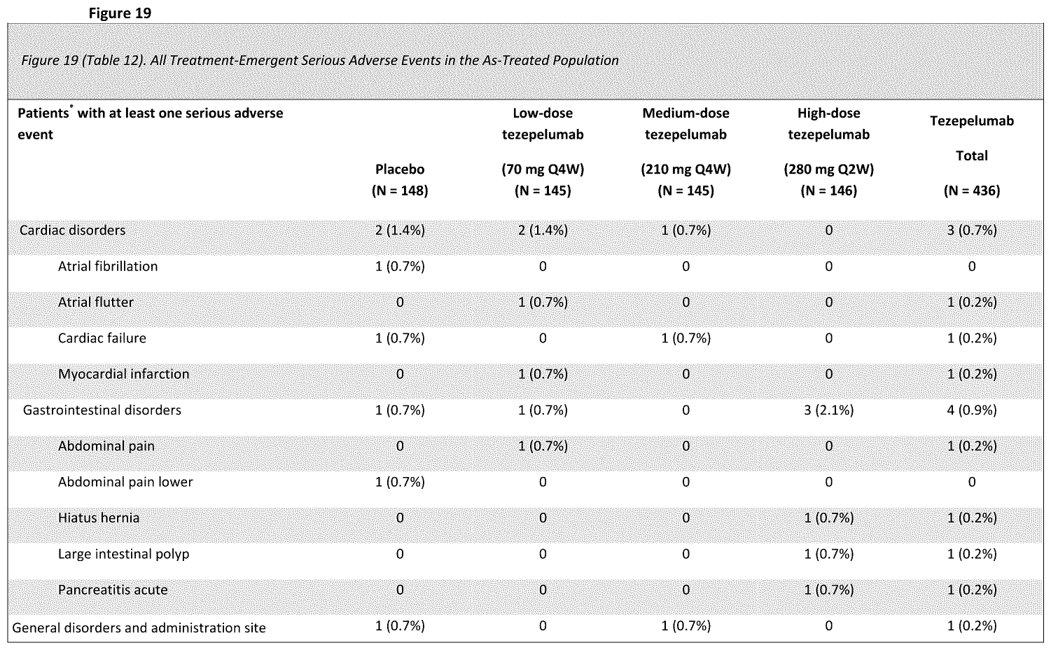

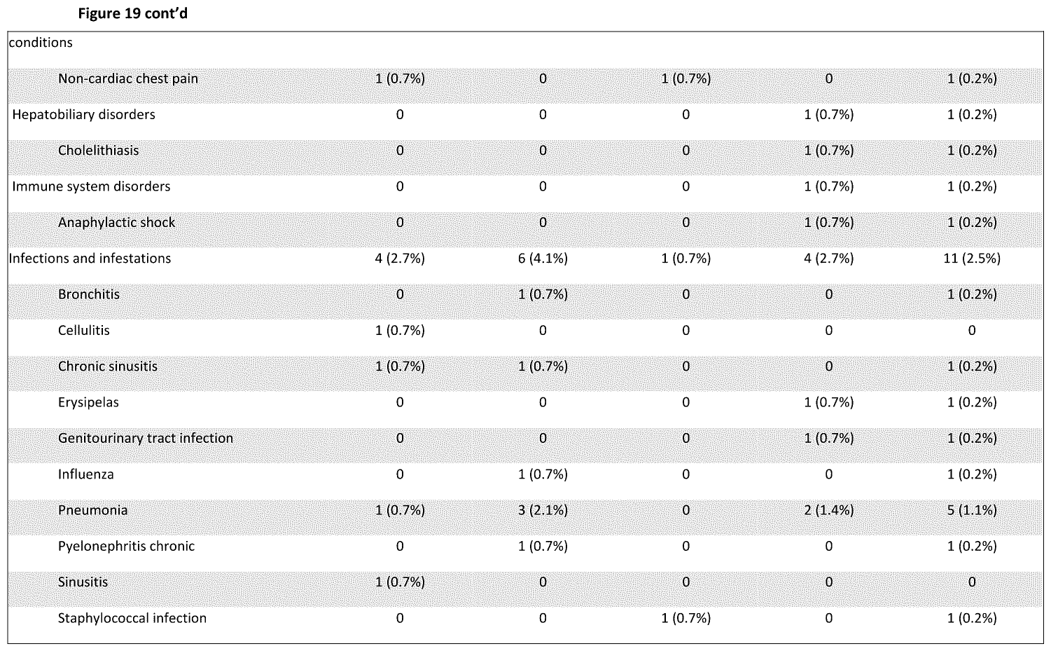

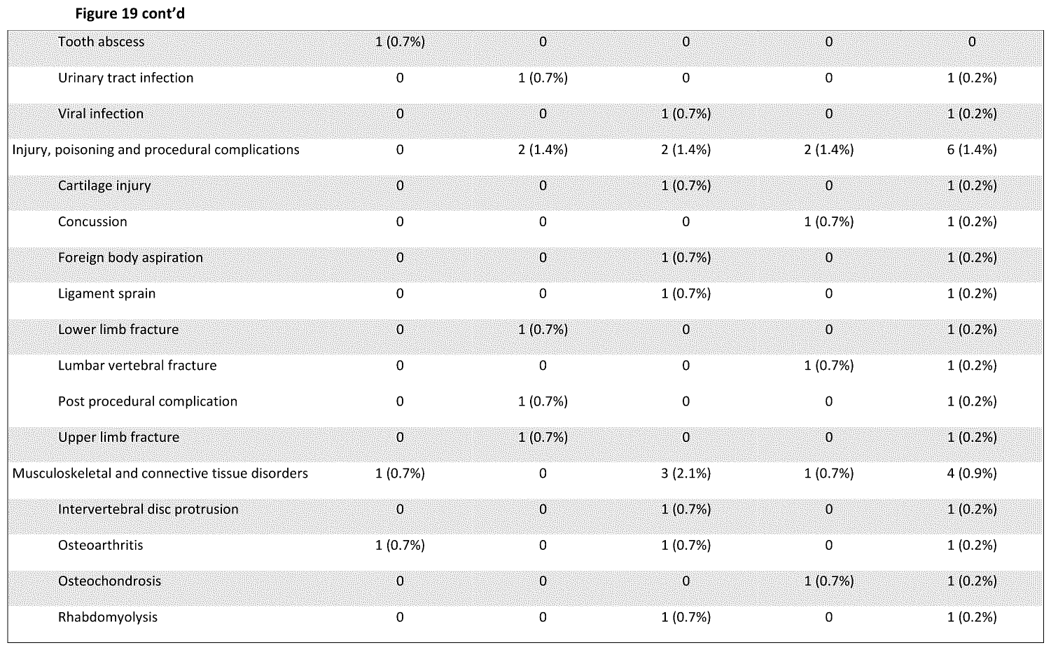

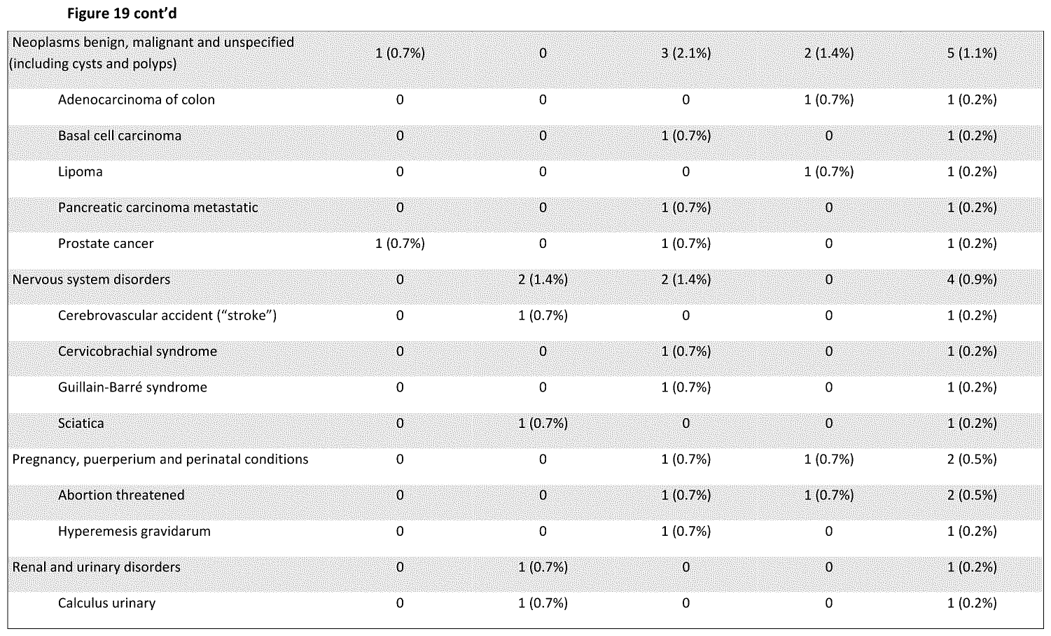

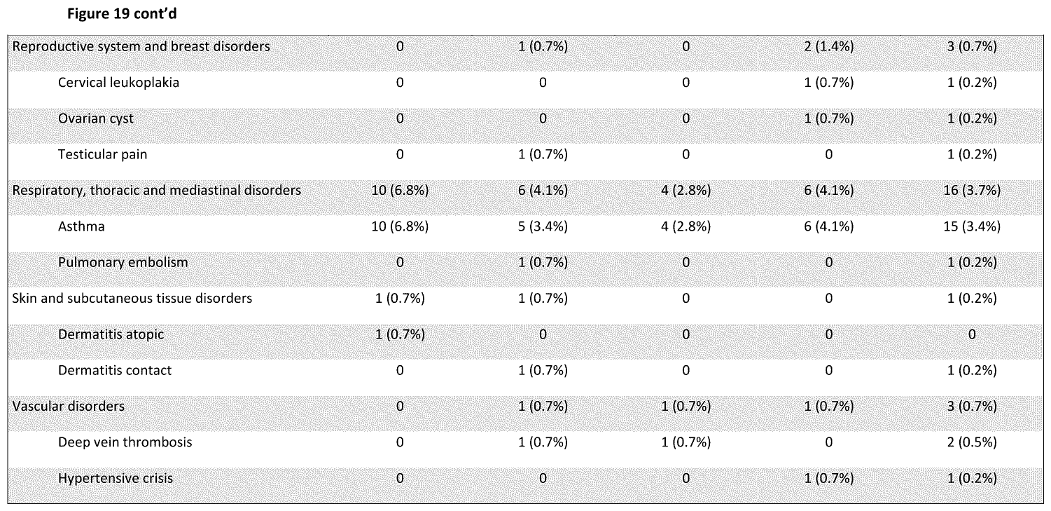

The annualized asthma exacerbation rate was lower in the tezepelumab groups than in the placebo group, irrespective of baseline eosinophil count or other assessed indicators of Th2 status (FIG. 2A; FIG. 6; Table 2; Table 4 and Tables 5, 7, 9 and 10). Among patients in the medium-dose inhaled glucocorticoid stratum, low-dose, medium-dose, and high-dose tezepelumab resulted in annualized asthma exacerbation rates at week 52 of 0.19, 0.14, and 0.20 events, respectively, as compared with 0.37 events with placebo. The rates in the tezepelumab groups were lower than the rate in the placebo group by 49% (95% Cl, −14 to 77; P=0.10), 62% (95% Cl, 8 to 84; P=0.03), and 47% (95% Cl, 41-20 to 76; P=0.13), respectively. Among patients in the high-dose inhaled glucocorticoid stratum, low-dose, medium-dose, and high-dose tezepelumab resulted in annualized asthma exacerbation rates at week 52 of 0.32, 0.23, and 0.24 events, respectively, as compared with 0.96 events with placebo. The rates in the tezepelumab groups were lower than the rate in the placebo group by 67% (95% Cl, 35 to 84; P=0.002), 76% (95% Cl, 49 to 89; P<0.001), and 75% (95% Cl, 47 to 88; P<0.001), respectively (Table 9). The annualized asthma exacerbation rate was lower in some, but not all, tezepelumab groups than in the placebo group when patients were stratified according to the number of asthma exacerbations in the previous 12 months and, in post hoc analyses, according to smoking history (Table 10).

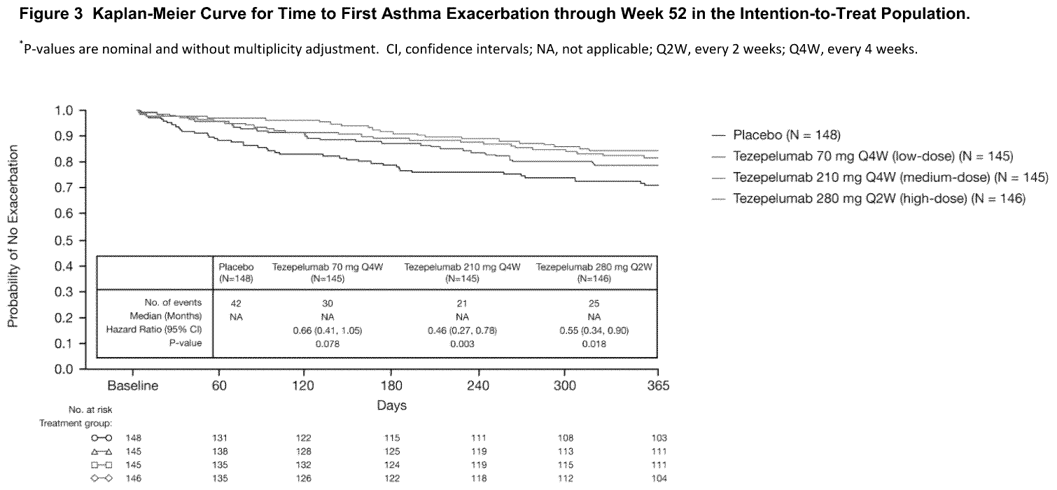

Time to first asthma exacerbation was longer in the tezepelumab groups than in the placebo group. The risk of having any exacerbation was lower in the low dose, medium dose and high dose tezepelumab groups than in the placebo group by 35% (hazard ratio [HR]0.65, 95% Cl 0.40, 1.04; P=0.07), 53% (HR 0.47, 95% Cl 0.28, 0.80; P=0.004), and 43% (HR 0.57, 95% Cl 0.35 to 0.93; P=0.02), respectively (FIG. 3 and Table 7)

In the overall population, the change from baseline at week 52 in the pre-BD FEV₁ was greater in the low dose, medium dose and high dose tezepelumab groups than in the placebo group by 0.12 L (95% Cl 0.02 to 0.21, P=0.01), 0.111 L (95% Cl 0.02, to 0.21, P=0.02), and 0.15 L (95% Cl 0.06, to 0.25, P=0.002), respectively (Table 2 and FIG. 1B). Similar differences were observed when the pre-BD FEV1 was measured as the percent of the predicted value (Table 2). The treatment effect was observed as early as week 4 (the first time point assessed) and was sustained for the duration of the trial (FIG. 1B, Table 2).

The effects of tezepelumab on additional secondary end points—including the percentage of patients with at least one asthma exacerbation, the percentage of patients with at least one severe asthma exacerbation, the annualized rate of severe asthma exacerbations, the time to the first severe asthma exacerbation and changes from baseline in the postbronchodilator FEV1, FVC, ACQ-6 score, AQLQ score, and asthma symptom score—are presented in Table 2, and FIGS. 1C and 1D and Tables 3, 5, 6 and 12. The effects of tezepelumab on secondary end points according to subgroup (prebronchodilator FEV1, ACQ-6 score, AQLQ score, and asthma symptom score) are shown in Tables 2, 4, 5 and 12.

### Biomarkers

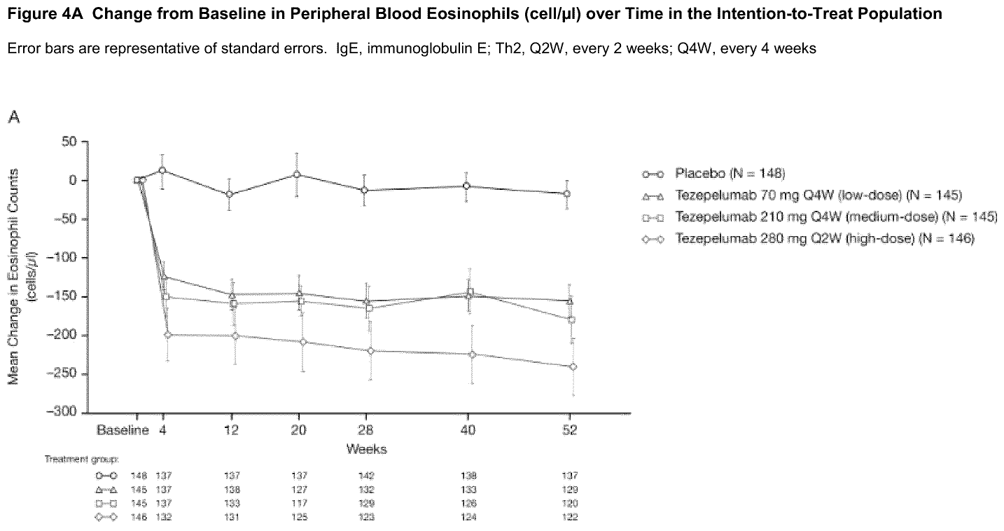

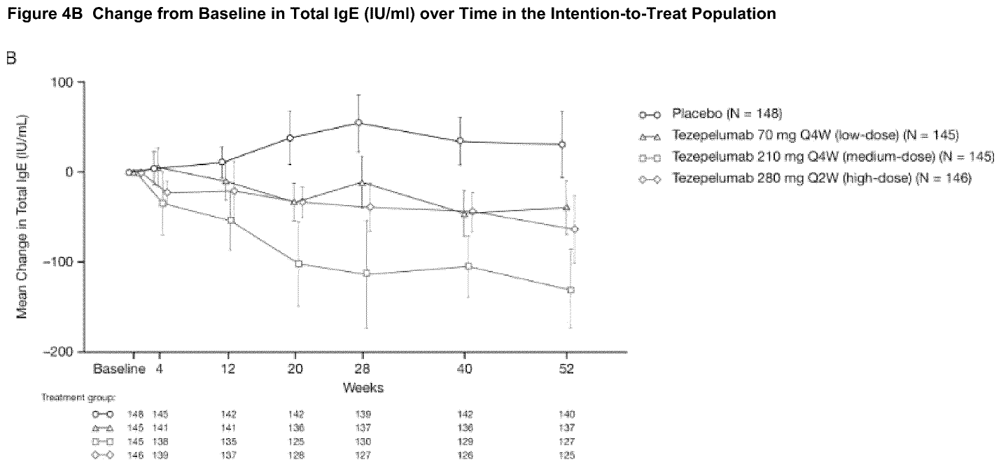

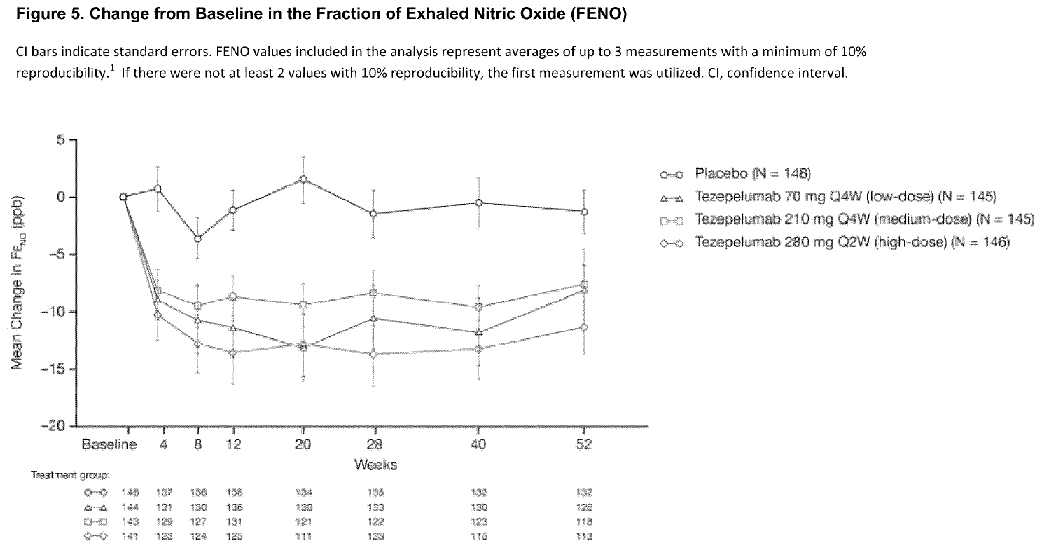

Substantial and persistent decreases in blood eosinophils and FeNO were observed in all tezepelumab treatment groups, beginning at week 4 (first time point assessed) after treatment initiation, and maintained over time (FIG. 4, FIG. 5, FIG. 6). Progressive decreases were also observed in total serum IgE in all tezepelumab groups (FIG. 4A).

### Safety and Tolerability

The overall subject incidence of AEs was similar across treatment groups (Table 3). In total, 62.2% of the patients in the placebo group, 65.5% of the patients in the low dose tezepelumab group, 64.1% of the patients in the medium dose tezepelumab group, and 61.6% of patients in the high dose tezepelumab group reported at least one adverse event, and 12.2%, 11.7%, 9.0%, and 12.3%, reported at least one serious adverse event, respectively. When asthma-related adverse events were removed from the above analysis, the overall incidence of adverse events was similar across the trial groups. A full list of serious adverse events is provided in Table 12.

Three serious adverse events to be related to the trial agent; two (pneumonia and stroke) occurred in the same patient in the low dose tezepelumab group and one (the Guillain-Barre syndrome) in the medium dose tezepelumab group. The rates of discontinuation due to adverse events were 1.1% among patients receiving tezepelumab (five patients, including two in the medium dose group and three in the high dose group) and [0.7% in the placebo group (one patient). One patient in the low dose tezepelumab group died 8 weeks after the treatment period ended from a treatment-related serious adverse event (stroke in the same patient described above).

Injection-site reactions after 1-mL injections occurred in 3.4% of the patients in the placebo group, 2.8% of the patients in the low-dose tezepelumab group, 2.8% of the patients in the medium-dose group, and 1.4% of the patients in the high-dose group. The rates after 1.5-mL injections were 2.7%, 2.1%, 2.8%, and 3.4% in the respective groups. No investigational product-related anaphylactic reactions were reported. After baseline, positive antidrug antibodies were noted in 13 of 148 patients (8.8%) in the placebo group, 7 of 144 patients (4.9%) in the low-dose tezepelumab group, 0 of 144 patients in the medium-dose group, and 3 of 143 patients (2.1%) in the high-dose group. No neutralizing antibodies were detected.

Analysis B: final analysis after database lock, includes all sites: For Analysis B, 145 patients were assigned to low dose tezepelumab (70 mg Q4W), 145 were assigned to medium dose tezepelumab (210 mg Q4W), 146 were assigned to high dose tezepelumab (280 mg Q2W) and 148 were assigned the placebo. Of the patients who received tezepelumab or placebo, and were included in the ITT population, 391 (89.7%) and 139 (93.9%) completed treatment, respectively. Baseline and clinical characteristics were similar across groups.

The dose range of inhaled glucocorticoids for patients at baseline for Analysis B is similar for Analysis A. The median dose was 400 μg per day of fluticasone administered by means of a dry-powder inhaler or equivalent in the medium-dose inhaled glucocorticoid stratum, with 73 patients in the placebo group, 71 in the low-dose tezepelumab group, 70 in the medium-dose group, and 72 in the high-dose group; and 1000 μg per day of fluticasone administered by means of a dry-powder inhaler or equivalent in the high-dose inhaled glucocorticoid stratum, with 75, 74, 75, and 74 patients in the respective trial groups.

### Primary Endpoint

Treatment with tezepelumab resulted in annualized rates of asthma exacerbations at week 52 of 0.26, 0.19, and 0.22 events in the low-dose, medium-dose, and high-dose groups, respectively, as compared with 0.67 events in the placebo group. Thus, exacerbation rates were lower in the tezepelumab groups than in the placebo group by 61% (90% confidence interval [Cl], 39 to 75; P<0.001), 71% (90% Cl, 53 to 82; P<0.001), and 66% (90% Cl, 47 to 79; P<0.001), respectively (Table 2, and FIG. 1A). The types of asthma exacerbations that were used for the primary analysis are described in Table 1B.

### Secondary Endpoints

The annualized asthma exacerbation rate was lower in the tezepelumab groups than in the placebo group, irrespective of baseline eosinophil count or other assessed indicators of Th2 status (FIG. 2A; Table 2; Table 4 and Tables 5, 7, 9 and 10). Among patients in the medium-dose inhaled glucocorticoid stratum, low-dose, medium-dose, and high-dose tezepelumab resulted in annualized asthma exacerbation rates at week 52 of 0.19, 0.15, and 0.20 events, respectively, as compared with 0.38 events with placebo. The rates in the tezepelumab groups were lower than the rate in the placebo group by 51% (95% Cl, −8 to 78; P=0.08), 60% (95% Cl, 5 to 83; P=0.04), and 49% (95% Cl, −13 to 77; P=0.10), respectively. Among patients in the high-dose inhaled glucocorticoid stratum, low-dose, medium-dose, and high-dose tezepelumab resulted in annualized asthma exacerbation rates at week 52 of 0.33, 0.23, and 0.24 events, respectively, as compared with 0.96 events with placebo. The rates in the tezepelumab groups were lower than the rate in the placebo group by 66% (95% Cl, 33 to 83; P=0.002), 76% (95% Cl, 49 to 89; P<0.001), and 75% (95% Cl, 47 to 88; P<0.001), respectively (Table 9). The annualized asthma exacerbation rate was lower in some, but not all, tezepelumab groups than in the placebo group when patients were stratified according to the number of asthma exacerbations in the previous 12 months and, in post hoc analyses, according to smoking history (Table 10).

Time to first asthma exacerbation was longer in the tezepelumab groups than in the placebo group. The risk of having any exacerbation was lower in the low dose, medium dose and high dose tezepelumab groups than in the placebo group by 34% (hazard ratio [HR]0.66, 95% Cl 0.41, 1.05; P=0.08), 54% (HR 0.46, 95% Cl 0.27, 0.78; P=0.003), and 45% (HR 0.55, 95% Cl 0.34 to 0.90; P=0.02), respectively (FIG. 3 and Table 7)

In the overall population, the change from baseline at week 52 in the pre-BD FEV1 was greater in the low dose, medium dose and high dose tezepelumab groups than in the placebo group by 0.12 L (95% Cl 0.02 to 0.21, P=0.01), 0.11 L (95% Cl 0.02, to 0.20, P=0.02), and 0.15 L (95% Cl 0.06, to 0.25, P=0.002), respectively (Table 2 and FIG. 1B). Similar differences were observed when the pre-BD FEV1 was measured as the percent of the predicted value (Table 2). The treatment effect was observed as early as week 4 (the first time point assessed) and was sustained for the duration of the trial (FIG. 1B, Table 2).

The effects of tezepelumab on additional secondary end points—including the percentage of patients with at least one asthma exacerbation, the percentage of patients with at least one severe asthma exacerbation, the annualized rate of severe asthma exacerbations, the time to the first severe asthma exacerbation and changes from baseline in the postbronchodilator FEV1, FVC, ACQ-6 score, AQLQ score, and asthma symptom score for Analysis B are consistent with those of Analysis A discussed above, and results in Table 2, and FIGS. 1C and 1D and Tables 3, 5, 6 and 12. The effects of tezepelumab on secondary end points according to subgroup (prebronchodilator FEV1, ACQ-6 score, AQLQ score, and asthma symptom score) are shown in Tables 2, 4, 5 and 12.

### Biomarkers

Substantial and persistent decreases in blood eosinophils and FeNO were observed in all tezepelumab treatment groups, beginning at week 4 (first time point assessed) after treatment initiation, and maintained over time (FIG. 2 and FIG. 4). Progressive decreases were also observed in total serum IgE in all tezepelumab groups (FIG. 2B).

### Safety and Tolerability

The overall subject incidence of AEs in Analysis B was consistent with Analysis A and was similar across treatment groups (Table 3). In total, 62.2% of the patients in the placebo group, 66.2% of the patients in the low dose tezepelumab group, 64.8% of the patients in the medium dose tezepelumab group, and 61.6% of patients in the high dose tezepelumab group reported at least one adverse event, and 12.2%, 11.7%, 9.0%, and 12.3%, reported at least one serious adverse event, respectively. When asthma-related adverse events were removed from the above analysis, the overall incidence of adverse events was similar across the trial groups. A full list of serious adverse events is provided in Table 12.

Three serious adverse events to be related to the trial agent; two (pneumonia and stroke) occurred in the same patient in the low dose tezepelumab group and one (Guillain-Barre syndrome) in the medium dose tezepelumab group. The rates of discontinuation due to adverse events were 1.1% among patients receiving tezepelumab (five patients, including two in the medium dose group and three in the high dose group) and 0.7% in the placebo group (one patient). One patient in the low dose tezepelumab group died 8 weeks after the treatment period ended from a treatment-related serious advserse event (stroke in the same patient described above).

For Analysis B, Injection-site reactions after 1-mL injections occurred in 3.4% of the patients in the placebo group, 2.8% of the patients in the low-dose tezepelumab group, 2.8% of the patients in the medium-dose group, and 1.4% of the patients in the high-dose group. The rates after 1.5-mL injections were 2.7%, 2.1%, 2.8%, and 3.4% in the respective groups. No investigational product-related anaphylactic reactions were reported. After baseline, positive antidrug antibodies were noted in 13 of 148 patients (8.8%) in the placebo group, 7 of 144 patients (4.9%) in the low-dose tezepelumab group, 1 of 140 patients (0.7%) in the medium-dose group, and 3 of 142 patients (2.1%) in the high-dose group. No neutralizing antibodies were detected.

In summary, the overall results of Analysis A and Analysis B were consistent.

Analysis C, after data lock, single site results omitted. Based on the study sponsor's concerns about data integrity at one clinical site enrolled in the Phase 2 study, the data from Analysis B after data lock was re-analyzed with patients from this site omitted. For this second analysis, patients who received tezepelumab or placebo, and were included in the ITT population, 367 (89.1%) and 129 (93.5%) completed treatment, respectively. Baseline and clinical characteristics were similar across groups. Analysis C is consistent with the results of the previous analysis.

For Analysis C, 138 patients were assigned to low dose tezepelumab (70 mg Q4W), 137 were assigned to medium dose tezepelumab (210 mg Q4W), 137 were assigned to high dose tezepelumab (280 mg Q2W) and 138 were assigned the placebo. Of the patients who received tezepelumab or placebo, and were included in the ITT population (excluding patients from the omitted site), 367 (89.1%) and 129 (93.5%) completed treatment, respectively. Baseline and clinical characteristics were similar across groups

The dose range of inhaled glucocorticoids for patients at baseline for Analysis B is similar for Analysis A and B, as shown in FIGS. 2A and 2B. The median dose was 400 μg per day of fluticasone administered by means of a dry-powder inhaler or equivalent in the medium-dose inhaled glucocorticoid stratum, with 73 patients in the placebo group, 67 in the low-dose tezepelumab group, 70 in the medium-dose group, and 71 in the high-dose group, and 1000 μg per day of fluticasone administered by means of a dry-powder inhaler or equivalent in the high-dose inhaled glucocorticoid stratum, with 65, 71, 67 and 66 patients in the respective trial groups.

### Primary Endpoint

Treatment with tezepelumab resulted in annualized rates of asthma exacerbations at week 52 of 0.27, 0.20, and 0.23 events in the low-dose, medium-dose, and high-dose groups, respectively, as compared with 0.72 events in the placebo group. Thus, exacerbation rates were lower in the tezepelumab groups than in the placebo group by 62% (90% confidence interval [Cl], 42 to 75; P<0.001), 71% (90% Cl, 54 to 82; P<0.001), and 66% (90% Cl, 47 to 79; P<0.001), respectively. The types of asthma exacerbations that were used for the primary analysis are described in Table 1B.

### Secondary Endpoints

The annualized asthma exacerbation rate was lower in the tezepelumab groups than in the placebo group, irrespective of baseline eosinophil count or other assessed indicators of Th2 status. Among patients in the medium-dose inhaled glucocorticoid stratum, low-dose, medium-dose, and high-dose tezepelumab resulted in annualized asthma exacerbation rates at week 52 of 0.20, 0.15, and 0.20 events, respectively, as compared with 0.38 events with placebo. The rates in the tezepelumab groups were lower than the rate in the placebo group by 48% (95% Cl, −15 to 76; P=0.11), 60% (95% Cl, 5 to 83; P=0.04), and 48% (95% Cl, −14 to 76; P=0.10), respectively. Among patients in the high-dose inhaled glucocorticoid stratum, low-dose, medium-dose, and high-dose tezepelumab resulted in annualized asthma exacerbation rates at week 52 of 0.35, 0.26, and 0.27 events, respectively, as compared with 1.12 events with placebo. The rates in the tezepelumab groups were lower than the rate in the placebo group by 70% (95% Cl, 41 to 84; P=<0.001), 77% (95% Cl, 52 to 89; P<0.001), and 76% (95% Cl, 50 to 88; P<0.001), respectively. The annualized asthma exacerbation rate was lower in some, but not all, tezepelumab groups than in the placebo group when patients were stratified according to the number of asthma exacerbations in the previous 12 months and, in post hoc analyses, according to smoking history.

Time to first asthma exacerbation was longer in the tezepelumab groups than in the placebo group. The risk of having any exacerbation was lower in the low dose, medium dose and high dose tezepelumab groups than in the placebo group by 38% (hazard ratio [HR]0.62, 95% Cl 0.39, 0.99; P=0.04), 55% (HR 0.45, 95% Cl 0.26, 0.75; P=0.002), and 46% (HR 0.54, 95% Cl 0.33 to 0.88; P=0.01), respectively.

In the analyzed population, the change from baseline at week 52 in the pre-BD FEV1 was greater in the low dose, medium dose and high dose tezepelumab groups than in the placebo group by 0.12 L (95% Cl 0.02 to 0.22, P=0.02), 0.13 L (95% Cl 0.03, to 0.23, P=0.01), and 0.15 L (95% Cl 0.05, to 0.25, P=0.002), respectively. Similar differences were observed when the pre-BD FEV1 was measured as the percent of the predicted value (Table 2). The treatment effect was observed as early as week 4 (the first time point assessed) and was sustained for the duration of the trial.

The effects of tezepelumab on additional secondary end points—including the percentage of patients with at least one asthma exacerbation, the percentage of patients with at least one severe asthma exacerbation, the annualized rate of severe asthma exacerbations, the time to the first severe asthma exacerbation and changes from baseline in the postbronchodilator FEV1, FVC, ACQ-6 score, AQLQ score, and asthma symptom score—for Analysis C are consistent with those of Analysis A and B discussed above. The effects of tezepelumab on secondary end points according to subgroup (prebronchodilator FEV1, ACQ-6 score, AQLQ score, and asthma symptom score) were also consistent with Analysis A and B above.

### Biomarkers

Substantial and persistent decreases in blood eosinophils and FeNO were observed in all tezepelumab treatment groups, beginning at week 4 (first time point assessed) after treatment initiation, and maintained over time. Progressive decreases were also observed in total serum IgE in all tezepelumab groups.

### Safety and Tolerability

The overall subject incidence of AEs in Analysis C was similar across treatment groups. In total, 65.9% of the patients in the placebo group, 67.4% of the patients in the low dose tezepelumab group, 65.7% of the patients in the medium dose tezepelumab group, and 65.0% of patients in the high dose tezepelumab group reported at least one adverse event, and 13.0%, 12.3%, 9.5%, and 13.1%, reported at least one serious adverse event, respectively. When asthma-related adverse events were removed from the above analysis, the overall incidence of adverse events was similar across the trial groups.

Three serious adverse events to be related to the trial agent; two (pneumonia and stroke) occurred in the same patient in the low dose tezepelumab group and one (the Guillain-Barre syndrome) in the medium dose tezepelumab group. The rates of discontinuation due to adverse events were 1.2% among patients receiving tezepelumab (five patients, including two in the medium dose group and three in the high dose group) and 0.7% in the placebo group (one patient). One patient in the low dose tezepelumab group died 8 weeks after the treatment period ended from a treatment-related serious adverse event (stroke in the same patient described above).

For Analysis C, injection-site reactions after 1-mL injections occurred in 3.6% of the patients in the placebo group, 2.9% of the patients in the low-dose tezepelumab group, 2.9% of the patients in the medium-dose group, and 1.5% of the patients in the high-dose group. The rates after 1.5-mL injections were 2.9%, 2.2%, 2.9%, and 3.6% in the respective groups. No investigational product-related anaphylactic reactions were reported. After baseline, positive antidrug antibodies were noted in 13 of 138 patients (9.4%) in the placebo group, 5 of 136 patients (3.7%) in the low-dose tezepelumab group, 1 of 131 patients (0.8%) in the medium-dose group, and 3 of 131 patients (2.3%) in the high-dose group. No neutralizing antibodies were detected.

In summary, the overall results of Analysis A, Analysis B and Analysis C were consistent.

Interestingly, a review of the effects of anti-TSLP treatment on the different high eosinophil and no eosinophil patients/low eosinophil patients showed that treatment with an anti-TSLP treatment was very effective in both high and low eosinophil patient populations, which would not have been expected in the low eosinophil population. Table 2 and FIG. 3 show that anti-TSLP treatment significantly reduced exacerbation rates in both eosinophil high and low populations.

Eosinophil cell levels in a subject are a marker for Th2 inflammation in a subject. In view of this association between eosinophils and Th2 levels, the study subjects were also divided into populations based on the relative Th2 levels at the start of treatment, e.g., Th2 high or low populations, and assayed for antibody efficacy. The results demonstrated that treatment with anti-TSLP was very effective in both Th2 high and Th2 low patient populations. Table 4 shows that anti-TSLP treatment significantly reduced exacerbation rates in both Th2 high and low populations, but to a greater extent in Th2 low patients.

### DISCUSSION

Treatment with tezepelumab resulted in significantly lower annualized rates of asthma exacerbations than the rate with placebo among patients whose asthma remained uncontrolled despite treatment with LABAs and medium- to high-doses of inhaled glucocorticoids. Some, but not all secondary outcomes were better with tezelpelumab than with placebo. Treatment effects were observed shortly after the initiation of treatment and were maintained throughout the trial. The incidence of adverse events was similar in the tezepelumab and placebo groups, with similar levels of discontinuations, regardless of asthma-related adverse events.

Tezepelumab reduced blood eosinophil counts, FeNO levels, and total serum IgE levels; changes in eosinophil counts and FeNO levels occurred rapidly from week 4 and concurrently with changes in clinical end points. These findings are consistent with the results from a previous allergen challenge study in patients with mild asthma, in which tezepelumab abrogated post-allergen challenge increases in sputum and blood eosinophils and FeNO.²⁴ These changes in biomarker levels demonstrate that TSLP is a key upstream regulator of Th2 activation and/or function, with effects on interleukin-4, interleukin-5, and interleukin-13 pathways, and indicate that inhibition of TSLP may have broader physiologic effects than individual Th2 cytokine inhibitors. Additionally, the epithelial-cell-derived cytokines interleukin-25 and interleukin-33 may work together with TSLP to initiate and amplify Th2 inflammation, although the interplay of these cytokines requires further investigation.^{32,33}

Tezepelumab was well-tolerated in all dose groups with no increase in reported infections compared with placebo.

The observed improvements in disease control following treatment with tezepelumab highlights the potential pathogenic role of TSLP across a range of asthma phenotypes. Non-allergic factors, including tobacco smoke, diesel particles and viruses, have been shown to trigger TSLP release and lead to activation of non-Th2 inflammatory responses in asthma.34-37 Cell types which are activated by TSLP and may participate in these pathways, include mast cells, basophils, natural killer T cells, group 2 innate lymphoid cells and possibly neutrophils and interleukin-17 cells.^{20,36-39}

The present data provides the first clinical evidence that inhibition of TSLP leads to a lower annualized rate of asthma exacerbations than no such inhibition, independent of baseline eosinophil count or other Th2 biomarkers and better results with respect to other clinical endpoints among patients with uncontrolled asthma who are receiving LABAs and medium-to-high doses of inhaled glucocorticoids. These findings highlight the potential advantages of targeting an upstream cytokine such as TSLP, which may affect disease activity more broadly than inhibition of a single downstream pathway.

Numerous modifications and variations of the invention as set forth in the above illustrative examples are expected to occur to those skilled in the art. Consequently only such limitations as appear in the appended claims should be placed on the invention.

### REFERENCES

1. To T, Stanojevic S, Moores G, et al. BMC Public Health 2012; 12:204.
2. Chung K F, Wenzel S E, Brozek J L, et al. Eur Respir J 2014; 43:343-73.
3. Pavord I D, et al., NPJ Prim Care Respir Med 2017; 27:17.
4. Bateman E D, et al. Am J Respir Crit Care Med 2004; 170:836-44.
5. GINA Report. Global strategy for asthma management and prevention. August 2014. http://www.ginaasthma.org2014.
6. Woodruff P G, et al. Am J Respir Crit Care Med 2009; 180:388-95.
7. Wenzel S E. Am J Respir Cell Mol Biol 2016; 55:1-4.
8. Froidure A, et al., Eur Respir J 2016; 47:304-19.
9. Swedin L, et al. Pharmacol Ther 2017; 169:13-34.
10. Brightling C, Berry M, Amrani Y. J Allergy Clin Immunol 2008; 121:5-10; quiz 1-2.
11. Ortega H G, Liu M C, Pavord I D, et al. N Engl J Med 2014; 371:1198-207.
12. XOLAIR® (omalizumab): Highlights of Prescribing Information 2016. (at https://www.gene.com/download/pdf/xolair_prescribing.pdf.)
13. Bleecker E R, FitzGerald J M, Chanez P, et al. The Lancet 2016; 388:2115-27.
14. FitzGerald J M, Bleecker E R, Nair P, et al. The Lancet 2016; 388:2128-41.
15. Wenzel S, Castro M, Corren J, et al. Lancet 2016; 388:31-44.
16. Castro M, et al. Lancet Respir Med 2015; Epub, doi: 10.1016/S2213-2600(15)00042-9.
17. Brightling C E, Chanez P, Leigh R, et al. Lancet Respir Med 2015; 3:692-701.
18. Bel E H, Wenzel S E, Thompson P J, et al. N Engl J Med 2014; 371:1189-97.
19. Soumelis V, Reche P A, Kanzler H, et al. Nat Immunol 2002; 3:673-80.
20. Allakhverdi Z, Comeau M R, Jessup H K, et al J Exp Med 2007; 204:253-8.
21. Shikotra A, Choy D F, Ohri C M, et al. J Allergy Clin Immunol 2012; 129:104-11 e1-9.
22. Ying S, O'Connor B, Ratoff J, et al. J Immunol 2005; 174:8183-90.
23. Ying S, O'Connor B, Ratoff J, et al. J Immunol 2008; 181:2790-8.
24. Gauvreau G M, O'Byrne P M, Boulet L P, et al. N Engl J Med 2014; 370:2102-10.
25. Juniper E F, O'Byrne P M, Guyatt G H, Ferrie P J, King D R. Eur Respir J 1999; 14:902-7.
26. Global Stratgey for Asthma Management and Prevention 2012. 2012, at http://ginasthma.org/.)
27. Juniper E F, Buist A S, Cox F M, Ferrie P J, King D R. Chest 1999; 115:1265-70.
28. Corren J, Lemanske R F, Hanania N A, et al. N Engl J Med 2011; 365:1088-98.
29. Dweik R A, Boggs P B, Erzurum S C, et al., Am J Respir Crit Care Med 2011; 184:602-15.
30. Hanania N A, Wenzel S, Rosen K, et al., Am J Respir Crit Care Med 2013; 187:804-11.
31. Tabrizi M, Bornstein G G, Suria H. AAPS J 2010; 12:33-43.
32. Paul W E, Zhu J. Nat Rev Immunol 2010; 10:225-35.
33. Gavala M L, Bashir H, Gern J E. Curr Allergy Asthma Rep 2013; 13:298-307.
34. Nakamura Y, Miyata M, Ohba T, et al. J Allergy Clin Immunol 2008; 122:1208-14.
35. Bleck B, et al., Journal of clinical immunology 2008; 28:147-56.
36. Lee H C, Headley M B, Loo Y M, et al. J Allergy Clin Immunol 2012; 130:1187-96 e5.
37. Calven J, Yudina Y, Hallgren O, et al. J Innate Immun 2012; 4:86-99.
38. Nagata Y, et al., Int Arch Allergy Immunol 2007; 144:305-14.
39. Kim B S, Siracusa M C, Saenz S A, et al. Sci Transl Med 2013; 5:170ra16.

What is claimed:

1. A method for treating asthma in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

2. A method for treating asthma in a subject comprising administering a therapeutically effective amount of an anti-TSLP antibody or antibody variant in a dose of 70 mg to 280 mg at an interval of every two weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

3. The method of any one of claim 1 or 2 wherein the antibody or antibody variant is administered every 4 weeks.

4. The method of any one of claims 1 to 3 wherein the antibody or antibody variant is administered at a dose of 70 mg.

5. The method of any one of claims 1 to 3 wherein the antibody or antibody variant is administered at a dose of 210 mg.

6. The method of any one of claims 1 to 3 wherein the antibody or antibody variant is administered at a dose of 280 mg.

7. A method for treating asthma in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

8. A method for treating asthma in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

9. The method of any one of the preceding claims wherein the antibody or antibody variant is administered for a period of at least 4 months, 6 months, 9 months, 1 year or more.

10. The method of any one of the preceding claims, wherein said anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

11. The method of claim 8, wherein said anti-TSLP antibody variant is selected from the group consisting of a diabody, a triabody, a tetrabody, a Fab fragment, single domain antibody, scFv, wherein the dose is adjusted such that the binding sites to be equimolar to the those dosed by bivalent antibodies.

12. The method of any one of the preceding claims, wherein the antibody is an IgG2 antibody.

13. The method of any one of the preceding claims, wherein the antibody or antibody variant is a human antibody.

14. The method of any one of the preceding claims wherein, the antibody or antibody variant further comprises a pharmaceutically acceptable carrier or excipient.

15. The method of any one of the preceding claims wherein, wherein the asthma is severe asthma.

16. The method of any one of the preceding claims, wherein the asthma is eosinophilic or non-eosinophilic asthma.

17. The method any one of the preceding claims, wherein the asthma is low eosinophil asthma.

18. The method of any one of the preceding claims, wherein the subject is an adult.

19. The method any one of the preceding claims, wherein the subject is a child or adolescent.

20. The method any one of the preceding claims, wherein the administration decreases eosinophils in blood, sputum, broncheoalveolar fluid, or lungs of the subject.

21. The method any one of the preceding claims, wherein the administration shifts cell counts in the subject from a Th2 high population to a Th2 low population.

22. The method any one of the preceding claims, wherein the administration improves one or more measures of asthma in a subject selected from the group consisting of forced expiratory volume (FEV), FEV₁ reversibility, forced vital capacity (FCV), FeNO, Asthma Control Questionnaire−6 score and AQLQ(S)+12 score.

23. The method any one of the preceding claims, wherein the administration improves one or more symptoms of asthma as measured by an asthma symptom diary.

24. A method for treating asthma in a subject comprising administering a therapeutically effective amount of an anti-TSLP antibody or antibody variant in a dose of 70 to 280 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2, wherein the antibody is an IgG2 antibody.

25. The method of claim 24 wherein the antibody is administered every 4 weeks.

26. The method of claim 24 or 25 wherein the antibody is administered at a dose of 70 mg.

27. The method of any one of claims 24 to 25 wherein the antibody is administered at a dose of 210 mg.

28. The method of any one of claims 24 to 25 wherein the antibody is administered at a dose of 280 mg.

29. The method of any one of the preceding claims wherein the antibody is tezepelumab.

30. The method of claim 29 wherein the antibody is an IgG2 antibody, and has the full length heavy and light chain sequences set out in SEQ ID NOs: 105 and 106, respectively.

31. The method of any one of the preceding claims wherein the antibody variant has substantially similar pK characteristics as tezepelumab in humans.

32. A method of reducing the frequency of asthma exacerbation in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

33. A method of reducing the frequency of asthma exacerbation in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

34. The method of any one of claim 32 or 33 wherein the antibody or antibody variant is administered every 4 weeks.

35. The method of any one of claim 32 or 33 wherein the antibody or antibody variant is administered at a dose of 70 mg.

36. The method of any one of claim 32 or 33 wherein the antibody or antibody variant is administered at a dose of 210 mg.

37. The method of any one of claim 32 or 33 wherein the antibody or antibody variant is administered at a dose of 280 mg.

38. The method of any one of claims 32 to 37 wherein the antibody or antibody variant is administered for a period of at least 4 months, 6 months, 9 months, 1 year or more.

39. The method of any one of claims 32 to 38, wherein said anti-TSLP antibody or antibody variant is selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

40. The method any one of claims 32 to 39, wherein antibody or antibody variant is an IgG2 antibody.

41. The method of any one of claims 32 to 40, wherein the antibody or antibody variant is a human antibody.

42. The method of any one of claims 32 to 41 wherein, the antibody or antibody variant further comprises a pharmaceutically acceptable carrier or excipient.

43. The method of any one of claims 32 to 42 wherein the administration delays the time to an asthma exacerbation compared to a subject not receiving the anti-TSLP antibody.

44. The method any one of claims 32 to 43 wherein the administration reduces frequency of or levels of co-administered therapy in the subject.

45. The method of claim 44 wherein the co-administered therapy is inhaled corticosteroids (ICS), long-acting β2 agonist (LABA), leukotriene receptor antagonists (LTRA), long-acting anti-muscarinics (LAMA), cromones, short-acting β2 agonist (SABA), and theophylline or oral corticosteroids.

46. The method of claim 44, wherein the administration eliminates the need for corticosteroid therapy.

47. A method of treating chronic obstructive pulmonary disease (COPD) comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

48. A method of treating chronic obstructive pulmonary disease (COPD) in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

49. The method any one of the preceding claims wherein the administration is subcutaneous or intravenous.

50. A method for reducing ACQ-6 score in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

51. A method for reducing ACQ-6 score in a subject comprising administering a therapeutically effective amount of an anti-TSLP antibody or antibody variant in a dose of 70 mg to 280 mg at an interval of every 2 weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

52. The method any one of the preceding claims wherein the administration is subcutaneous or intravenous.

53. A method for treating asthma in a subject having a non-eosinophilic profile or a low eosinophil profile comprising administering a therapeutically effective amount of an anti-TSLP antibody or antibody variant, wherein the antibody or antibody variant binding to TSLP inhibits TSLP activity.

54. The method of claim 53 wherein the anti-TSLP antibody or anti-TSLP antibody variant is selected from an antibody described in Table A:
TABLE A

| | |
|---|---|
| WO2017/042701 | An anti-TSLP antibody comprising a heavy chain (HC) CDR1 |
| | comprising the sequence of SEQ ID NO: 13, a HC CDR2 |
| | comprising the sequence of SEQ ID NO: 14, and a HC CDR3 |
| | comprising the sequence of SEQ ID NO: 15; |
| | An anti-TSLP antibody comprising a light chain (LC) CDR1 |
| | comprising the sequence of SEQ ID NO: 16, a LC CDR2 |
| | comprising the sequence of SEQ ID NO: 17, a LC CDR3 |
| | comprising the sequence of SEQ ID NO: 18; |
| | An anti-TSLP antibody comprising a heavy chain (HC) CDR1 |
| | comprising the sequence of SEQ ID NO: 19, a HC CDR2 |
| | comprising the sequence of SEQ ID NO: 20, a HC CDR3 |
| | comprising the sequence of SEQ ID NO: 15; |
| | An anti-TSLP antibody comprising a light chain (LC) CDR1 |
| | comprising the sequence of SEQ ID NO: 21, a LC CDR2 |
| | comprising the sequence of SEQ ID NO: 22, a LC CDR3 |
| | comprising the sequence of SEQ ID NO: 23; |
| | An anti-TSLP antibody comprising a HC variable region comprising |
| | the sequence of SEQ ID NO: 26 and/or a LC variable region |
| | comprising the sequence of SEQ ID NO: 27; |
| | An anti-TSLP antibody comprising a HC variable region comprising |
| | the sequence of SEQ ID NO: 28 and/or a LC variable region |
| | comprising the sequence of SEQ ID NO: 29; |
| | An anti-TSLP antibody that comprises a paratope comprising at |
| | least one of the following residues: Thr28, Asp31, Tyr32, Trp33, |
| | Asp56, Glu101, Ile102, Tyr103, Tyr104, Tyr105 of a heavy chain |
| | sequence of SEQ ID NO: 26 or Gly28, Ser29, Lys30, Tyr31, Tyr48, |
| | Asp50, Asn51, Glu52, Asn65, and Trp92 of a light chain sequence |
| | of SEQ ID NO: 27; |
| | An anti-TSLP antibody that specifically binds an epitope in human |
| | TSLP, wherein the epitope comprises at least one of the following |
| | residues: Lys38, Ala41, Leu44, Ser45, Thr46, Ser48, Lys49, Ile52, |
| | Thr53, Ser56, Gly57, Thr58, Lys59, Lys101, Gln145, and Arg149 of |
| | SEQ ID NO: 30; |
| WO2016/142426 | An anti-TSLP antibody comprising the amino acid sequence of SEQ |
| | ID NO: 31; |
| | An anti-TSLP antibody comprising a CDR1 comprising the |
| | sequence of SEQ ID NO: 32; a CDR2 comprising the sequence of |
| | SEQ ID NO: 33, and a CDR3 comprising the sequence of SEQ ID |
| | NO: 34; |
| | An anti-TSLP antibody comprising a CDR1 comprising the |
| | sequence of SEQ ID NO: 32; a CDR2 comprising the sequence of |
| | SEQ ID NO: 35, and a CDR3 comprising the sequence of SEQ ID |
| | NO: 34; |
| | An anti-TSLP antibody comprising a variant of the CDR1 of SEQ ID |
| | NO: 31 wherein the residue corresponding to residue 28 in SEQ ID |
| | NO: 31 is Pro, the residue corresponding to residue 30 in SEQ ID |
| | NO: 31 is Arg, the residue corresponding to residue 31 in SEQ ID |
| | NO: 31 is Asn, the residue corresponding to residue 32 in SEQ ID |
| | NO: 31 is Trp and the residue corresponding to residue 34 in SEQ |
| | ID NO: 31 is Asp; |
| | An anti-TSLP antibody comprising a variant of the CDR2 of SEQ ID |
| | NO: 31 wherein the residue corresponding to residue 50 in SEQ ID |
| | NO: 31 is Gly, the residue corresponding to residue 53 in SEQ ID |
| | NO: 31 is His and the residue corresponding to residue 55 in SEQ |
| | ID NO: 31 is Gln; |
| | An anti-TSLP antibody comprising a variant of the CDR3 of SEQ ID |
| | NO: 31 wherein the residue corresponding to residue 91 in SEQ ID |
| | NO: 31 is He, Leu, Val or Phe, the residue corresponding to residue |
| | 92 in SEQ ID NO: 31 is Gly or Ala, the residue corresponding to |
| | residue 93 in SEQ ID NO: 31 is Glu, Phe, Asp or Ser and the |
| | residue corresponding to residue 94 in SEQ ID NO: 31 is Asp. |
| WO2010/017468 | An anti-TSLP antibody (9B7) comprising a HC CDR3 comprising |
| | the sequence of SEQ ID NO: 38, wherein the other CDRs of the HC |
| | and LC comprise the sequences of SEQ ID NOs: 36, 37, and 39-41; |
| | An anti-TSLP antibody (6C5) comprising a HC CDR3 comprising |
| | the sequence of SEQ ID NO: 44, wherein the other CDRs of the HC |
| | and LC comprise the sequences of SEQ ID NOs: 42, 43, and 45-47; |
| | An anti-TSLP antibody (6A3) comprising a HC CDR3 comprising |
| | the sequence of SEQ ID NO: 50, wherein the other CDRs of the HC |
| | and LC comprise the sequences of SEQ ID NOs: 48, 49, and 51-53; |
| | An anti-TSLP antibody (1A11) comprising a HC CDR3 comprising |
| | the sequence of SEQ ID NO: 56, wherein the other CDRs of the HC |
| | and LC comprise the sequences of SEQ ID NOs: 54, 55, and 57-59; |
| | An anti-TSLP antibody comprising (i) heavy chain variable region of |
| | SEQ ID NO: 60 and/or the light chain variable region of SEQ ID |
| | NO: 61; |
| | An anti-TSLP antibody comprising (i) heavy chain variable region of |
| | SEQ ID NO: 62 and/or the light chain variable region of SEQ ID |
| | NO: 63; |
| | An anti-TSLP antibody comprising (i) heavy chain variable region of |
| | SEQ ID NO: 64 and/or the light chain variable region of SEQ ID |
| | NO: 65; |
| | An anti-TSLP antibody comprising (i) heavy chain variable region of |
| | SEQ ID NO: 66 and/or the light chain variable region of SEQ ID NO: 67; |
| | An anti-TSLP antibody comprising (i) heavy chain variable region of |
| | SEQ ID NO: 68 and/or the light chain variable region of SEQ ID NO: 69; |
| | An anti-TSLP antibody comprising a HC CDR selected from the |
| | group consisting of SEQ ID NO: 38, SEQ ID NO: 44, SEQ ID NO: 50 |
| | and SEQ ID NO: 56, and analogs thereof; |
| | An anti-TSLP antibody comprising a heavy chain comprising the |
| | following CDRs or analogs thereof CDRH1: RYNVH (SEQ ID |
| | NO: 36), CDRH2: MIWDGGSTDYNSALKS (SEQ ID NO: 37), |
| | CDRH3: NRYESG (SEQ ID NO: 38), and a light chain comprising |
| | the following CDRs or analogs thereof CDRL1: |
| | KSSQSLLNSGNRKNYLT (SEQ ID NO: 39), CDRL2: WASTRES |
| | (SEQ ID NO: 40), and CDRL3: QNDYTYPFTFGS (SEQ ID NO: 41); or |
| | An anti-TSLP antibody comprising a heavy chain comprising the |
| | following CDRs or analogs thereof CRDH1: AYWMS (SEQ ID |
| | NO: 42), CDRH2: EINPDSSTINCTPSLKD (SEQ ID NO: 43), CDRH3: |
| | RLRPFWYFDVW (SEQ ID NO: 44), and a light chain comprising the |
| | following CDRs or analogs thereof CDRL1: |
| | RSSQSIVQSNGNTYLE (SEQ ID NO: 45), CDRL2: KVSNRFS (SEQ |
| | ID NO: 46), and CDRL3: FQGSHVPRT (SEQ ID NO: 47); |
| | An anti-TSLP antibody comprising a heavy chain comprising the |
| | following CDRs or analogs thereof CRDH1: TDYAWN (SEQ ID |
| | NO: 48), CDRH2: YIFYSGSTTYTPSLKS (SEQ ID NO: 49), CDRH3: |
| | GGYDVNYF (SEQ ID NO: 50), and a light chain comprising the |
| | following CDRs or analogs thereof CDRL1: LASQTIGAWLA (SEQ |
| | ID NO: 51), CDRL2: AATRLAD (SEQ ID NO: 52), and CDRL3: |
| | QQFFSTPWT (SEQ ID NO: 53); |
| | An anti-TSLP antibody comprising a heavy chain comprising the |
| | following CDRs or analogs thereof CDRH1: GYTMN (SEQ ID |
| | NO: 54), CDRH2: LINPYNGVTSYNQKFK (SEQ ID NO: 55), CDRH3: |
| | GDGNYWYF (SEQ ID NO: 56), and a light chain comprising the |
| | following CDRs or analogs thereof CDRL1: SASSSVTYMHW (SEQ |
| | ID NO: 57), CDRL2: EISKLAS (SEQ ID NO: 58), and CDRL3: |
| | QEWNYPYTF (SEQ ID NO: 59); |
| | An anti-TSLP antibody comprising a HC CDR1 comprising the |
| | sequence of SEQ ID NO: 70; a CDR2 comprising the sequence of |
| | SEQ ID NO: 71 , and a CDR3 comprising the sequence of SEQ ID |
| | NO: 72; |
| | An anti-TSLP antibody comprising a LC CDR1 comprising the |
| | sequence of SEQ ID NO: 73; a CDR2 comprising the sequence of |
| | SEQ ID NO: 74, and a CDRS comprising the sequence of SEQ ID |
| | NO: 75; |
| US2012/0020988 | An anti-TSLP antibody comprising a heavy chain variable domain |
| | comprising a CDR1 region of SEQ ID NO: 76, a CDR2 region of |
| | SEQ ID NO: 77, and CDR3 region of SEQ ID NO: 78, and a light |
| | chain variable domain comprising a CDR1 region of SEQ ID NO: |
| | 79, a CDR2 region of SEQ ID NO: 80, and a CDR3 region of SEQ |
| | ID NO: 81. |
| | An anti-TSLP antibody comprising a heavy chain variable domain |
| | comprising SEQ ID NO: 82 and a light chain variable domain |
| | comprising SEQ ID NO: 83; |
| | An anti-TSLP antibody comprising a heavy chain variable domain |
| | comprising a CDR1 region of SEQ ID NO: 76 or 84, a CDR2 region |
| | of SEQ ID NO: 77 or 85, and CDR3 region of SEQ ID NO: 78, and a |
| | light chain variable domain comprising a CDR1 region of SEQ ID |
| | NO: 79 or 86, a CDR2 region of SEQ ID NO: 80, 87, or 88, and a |
| | CDR3 region of SEQ ID NO: 81. |
| | An anti-TSLP antibody comprising a heavy chain variable domain |
| | comprising a CDR1 region of SEQ ID NO: 76, a CDR2 region of |
| | SEQ ID NO: 85, and CDR3 region of SEQ ID NO: 78, and a light |
| | chain variable domain comprising a CDR1 region of SEQ ID NO: |
| | 86, a CDR2 region of SEQ ID NO: 87 and a CDR3 region of SEQ ID |
| | NO: 81; |
| | An anti-TSLP antibody comprising a heavy chain variable domain |
| | comprising a CDR1 region of SEQ ID NO: 76, a CDR2 region of |
| | SEQ ID NO: 85, and CDR3 region of SEQ ID NO: 78, and a light |
| | chain variable domain comprising a CDR1 region of SEQ ID NO: |
| | 86, a CDR2 region of SEQ ID NO: 88 and a CDR3 region of SEQ ID |
| | NO: 81; |
| | An anti-TSLP antibody comprising a heavy chain variable domain |
| | comprising a CDR1 region of SEQ ID NO: 84, a CDR2 region of |
| | SEQ ID NO: 85, and CDR3 region of SEQ ID NO: 78, and a light |
| | chain variable domain comprising a CDR1 region of SEQ ID NO: |
| | 86, a CDR2 region of SEQ ID NO: 88 and a CDR3 region of SEQ ID |
| | NO: 81; or |
| | An anti-TSLP antibody comprising a heavy chain variable domain |
| | comprising a CDR1 region of SEQ ID NO: 76, a CDR2 region of |
| | SEQ ID NO: 85, and CDR3 region of SEQ ID NO: 78, and a light |
| | chain variable domain comprising a CDR1 region of SEQ ID NO: |
| | 86, a CDR2 region of SEQ ID NO: 80 and a CDR3 region of SEQ |
| | ID NO: 81. |
| | An anti-TSLP antibody comprising a heavy chain variable domain |
| | comprises SEQ ID NO: 89 and a light chain variable domain |
| | comprises SEQ ID NO: 90; |
| | An anti-TSLP antibody comprising a heavy chain variable domain |
| | comprises SEQ ID NO: 89 and a light chain variable domain |
| | comprises SEQ ID NO: 91; |
| | An anti-TSLP antibody comprising a heavy chain variable domain |
| | comprises SEQ ID NO: 92 and a light chain variable domain |
| | comprises SEQ ID NO: 93; |
| | An anti-TSLP antibody comprising a heavy chain variable domain |
| | comprises SEQ ID NO: 89 and a light chain variable domain |
| | comprises SEQ ID NO: 94, |
| U.S. Pat. No. | An anti-TSLP antibody comprising heavy chain variable region |
| 8,637,019 | comprising: a CDR-H1 sequence comprising SEQ ID NO: 95, a |
| | CDR-H2 sequence comprising SEQ ID NO: 96, and a CDR-H3 |
| | sequence comprising SEQ ID NO: 97; and/or an antibody light chain |
| | variable region or a TSLP-binding fragment thereof, said light chain |
| | variable region comprising: a CDR-L1 sequence comprising SEQ ID |
| | NO: 98, a CDR-L2 sequence comprising SEQ ID NO: 99, and a |
| | CDR-L3 sequence comprising SEQ ID NO: 100. |
| | An anti-TSLP antibody comprising a heavy chain variable region |
| | comprises the amino acid sequence of SEQ ID NO: 101 and the |
| | light chain variable region comprises the amino acid sequence of |
| | SEQ ID NO: 102. |
| | An anti-TSLP antibody comprising SEQ ID NO: 103 and SEQ ID |
| | NO: 104. |

55. The method of claim any one of claims 32 to 54 wherein the anti-TSLP antibody is tezepelumab.

56. The method of claim 55 wherein the antibody is an IgG2 antibody, and has the full length heavy and light chain sequences set out in SEQ ID NOs: 105 and 106, respectively.

57. The method of any one of claims 32 to 56 wherein the antibody variant has substantially similar pK characteristics as tezepelumab in humans.

58. The method of any one of claims 53, 54, or 55 wherein the subject has an eosinophil count less than 250 cells/μL at start of treatment.

59. A method for treating asthma in a subject having a Th2 low profile comprising administering a therapeutically effective amount of an anti-TSLP antibody or antibody variant, wherein the antibody or antibody variant binding to TSLP inhibits TSLP activity.

60. The method of claim 59 wherein the subject has a Th2 profile of IgE less than or equal to 100 IU/ml or eosinophil count of less than 140 cells/μL at the time of diagnosis.

61. The methods of claim 59 or 60 wherein the antibody or antibody variant is selected from an antibody described in Table A.

62. The method of claim 59 or 60 where in the antibody is tezepelumab.

63. The method of claim 62 wherein the antibody is an IgG2 antibody, and has the full length heavy and light chain sequences set out in SEQ ID NOs: 105 and 106, respectively.

64. A method for reducing ACQ-6 score in a subject having a low eosinophil profile comprising administering a therapeutically effective amount of an anti-TSLP antibody or antibody variant, wherein the antibody or antibody variant binding to TSLP inhibits TSLP activity.

65. The method of claim 64 wherein the anti-TSLP antibody or anti-TSLP antibody variant is selected from an antibody described in Table A.

66. The method of claim 64 wherein the anti-TSLP antibody is tezepelumab.

67. The method of claim 66 wherein the antibody is an IgG2 antibody, and has the full length heavy and light chain sequences set out in SEQ ID NOs: 105 and 106, respectively.

68. The method of claims 64, 65, or 66 wherein the subject has an eosinophil count less than 250 cells/μL at start of treatment.

69. A method for reducing ACQ-6 score in a subject having a Th2 low profile comprising administering a therapeutically effective amount of an anti-TSLP antibody or antibody variant, wherein the antibody or antibody variant binding to TSLP inhibits TSLP activity.

70. The method of claim 69 wherein the subject has a Th2 profile of IgE less than or equal to 100 IU/ml or eosinophil count of less than 140 cells/μL at the time of diagnosis.

71. The methods of claim 69 or 70 wherein the antibody or antibody variant is selected from an antibody described in Table A.

72. The method of claim 69 or 70 where in the antibody is tezepelumab.

73. The method of claim 72 wherein the antibody is an IgG2 antibody, and has the full length heavy and light chain sequences set out in SEQ ID NOs: 105 and 106, respectively.

74. The method of any one of claims 53-73 wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

75. The method of any one of claims 53-73 wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

76. The method of any one of claims 53-75 wherein the antibody or antibody variant is administered every 4 weeks.

77. The method any one of claims 53-76 wherein the administration is subcutaneous or intravenous.


SEQUENCE LISTING (106 sequences)

SEQ ID NO: 1 - DNA, 743 bp, Homo sapiens
  misc_feature 1..743: note = TSLP
  source 1..743: mol_type = genomic DNA
  CDS 200..676
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, Homo sapiens
  REGION 1..11: note = MISC_FEATURE - LCDR1
GGNNLGSKSVH

SEQ ID NO: 4 - protein, 7 aa, Homo sapiens
  REGION 1..7: note = MISC_FEATURE - LCDR2
DDSDRPS

SEQ ID NO: 5 - protein, 11 aa, Homo sapiens
  REGION 1..11: note = MISC_FEATURE - LCDR3
QVWDSSSDHVV

SEQ ID NO: 6 - protein, 5 aa, Homo sapiens
  REGION 1..5: note = MISC_FEATURE - HCDR1
TYGMH

SEQ ID NO: 7 - protein, 17 aa, Homo sapiens
  REGION 1..17: note = MISC_FEATURE - HCDR2
VIWYDGSNKHYADSVKG

SEQ ID NO: 8 - protein, 13 aa, Homo sapiens
  REGION 1..13: note = MISC_FEATURE - HCDR3
APQWELVHEAFDI

SEQ ID NO: 9 - DNA, 366 bp, Homo sapiens
  misc_feature 1..366: note = Heavy Chain VH
  source 1..366: mol_type = genomic DNA
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 122 aa, Homo sapiens
  REGION 1..122: note = MISC_FEATURE - Heavy Chain VH
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, Homo sapiens
  misc_feature 1..325: note = Light Chain VL
  source 1..325: mol_type = genomic DNA
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 108 aa, Homo sapiens
  REGION 1..108: note = MISC_FEATURE - Light Chain VL
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 13 - protein, 5 aa, synthetic construct
  REGION 1..5: note = Synthetic peptide
DYWMH

SEQ ID NO: 14 - protein, 19 aa, synthetic construct
  REGION 1..19: note = Synthetic peptide
HIKSKTDAGTTDYAAPVKG

SEQ ID NO: 15 - protein, 9 aa, synthetic construct
  REGION 1..9: note = Synthetic peptide
EIYYYAFDS

SEQ ID NO: 16 - protein, 11 aa, synthetic construct
  REGION 1..11: note = Synthetic peptide
SGDNIGSKYVH

SEQ ID NO: 17 - protein, 7 aa, synthetic construct
  REGION 1..7: note = Synthetic peptide
GDNERPS

SEQ ID NO: 18 - protein, 10 aa, synthetic construct
  REGION 1..10: note = Synthetic peptide
QAADWVDFYV

SEQ ID NO: 19 - protein, 7 aa, synthetic construct
  REGION 1..7: note = Synthetic peptide
GFTFSDY

SEQ ID NO: 20 - protein, 8 aa, synthetic construct
  REGION 1..8: note = Synthetic peptide
KSKTDAGT

SEQ ID NO: 21 - protein, 7 aa, synthetic construct
  REGION 1..7: note = Synthetic peptide
DNIGSKY

SEQ ID NO: 22 - skipped

SEQ ID NO: 23 - protein, 7 aa, synthetic construct
  REGION 1..7: note = Synthetic peptide
ADWVDFY

SEQ ID NO: 24 - protein, 120 aa, synthetic construct
  REGION 1..120: note = Synthetic peptide
EVQLVESGGGLVKPGGSLRLSCAASGFTFSDYWMHWVRQAPGKGLEWVGHIKSKTDAGTTDYAAPVKGRFTISRDDSKNTLYLQMNSLKTEDTAVYYCAREIYYYAFDSWGQGTLVTVSS

SEQ ID NO: 25 - protein, 107 aa, synthetic construct
  REGION 1..107: note = Synthetic peptide
SYELTQPLSVSVALGQTARITCSGDNIGSKYVHWYQQKPGQAPVLVIYGDNERPSGIPERFSGSNSGNTATLTISRAQAGDEADYYCQAADWVDFYVFGGGTKLTVL

SEQ ID NO: 26 - protein, 223 aa, synthetic construct
  REGION 1..223: note = Synthetic peptide
EVQLVESGGGLVKPGGSLRLSCAASGFTFSDYWMHWVRQAPGKGLEWVGHIKSKTDAGTTDYAAPVKGRFTISRDDSKNTLYLQMNSLKTEDTAVYYCAREIYYYAFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 27 - protein, 213 aa, synthetic construct
  REGION 1..213: note = Synthetic peptide
SYELTQPLSVSVALGQTARITCSGDNIGSKYVHWYQQKPGQAPVLVIYGDNERPSGIPERFSGSNSGNTATLTISRAQAGDEADYYCQAADWVDFYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 450 aa, synthetic construct
  REGION 1..450: note = Synthetic peptide
EVQLVESGGGLVKPGGSLRLSCAASGFTFSDYWMHWVRQAPGKGLEWVGHIKSKTDAGTTDYAAPVKGRFTISRDDSKNTLYLQMNSLKTEDTAVYYCAREIYYYAFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 213 aa, synthetic construct; identical to SEQ ID NO: 27
  REGION 1..213: note = Synthetic peptide

SEQ ID NO: 30 - protein, 149 aa, synthetic construct
  REGION 1..149: note = Synthetic peptide
MGSSHHHHHHLEVLFQGPYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 31 - protein, 107 aa, synthetic construct
  REGION 1..107: note = Dom30h-440-81/86 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCVQIGEDPVTFGQGTKVEIK

SEQ ID NO: 32 - protein, 11 aa, synthetic construct
  REGION 1..11: note = CDRL1 of Dom30h-440-81/86, Dom30h-440-53, Dom30h-440-54, Dom30h-440-55, Dom30h-440-56, Dom30h-440-57, Dom30h-440-58, Dom30h-440-60, Dom30h-440-63, Dom30h-440-64 and Dom30h-440-65 (Kabat, Chothia and AbM CDR definition)
RASRPIRNWLD

SEQ ID NO: 33 - protein, 7 aa, synthetic construct
  REGION 1..7: note = CDRL2 of Dom30h-440-81/86, Dom30h-440-53, Dom30h-440-54, Dom30h-440-55, Dom30h-440-56, Dom30h-440-57, Dom30h-440-58, Dom30h-440-60, Dom30h-440-63, Dom30h-440-64 and Dom30h-440-65 (Kabat, Chothia, AbM CDR definition)
GASHLQS

SEQ ID NO: 34 - protein, 9 aa, synthetic construct
  REGION 1..9: note = CDRL3 of Dom30h-440-81/86 and Dom30h-440-55 (Kabat, Chothia, AbM CDR definition)
VQIGEDPVT

SEQ ID NO: 35 - protein, 10 aa, synthetic construct
  REGION 1..10: note = CDRL2 of Dom30h-440-81/86 (Contact CDR definition)
LLIWGASHLQ

SEQ ID NO: 36 - protein, 5 aa, Mus musculus
RYNVH

SEQ ID NO: 37 - protein, 16 aa, Mus musculus
MIWDGGSTDYNSALKS

SEQ ID NO: 38 - protein, 6 aa, Mus musculus
NRYESG

SEQ ID NO: 39 - protein, 17 aa, Mus musculus
KSSQSLLNSGNRKNYLT

SEQ ID NO: 40 - protein, 7 aa, Mus musculus
WASTRES

SEQ ID NO: 41 - protein, 12 aa, Mus musculus
QNDYTYPFTFGS

SEQ ID NO: 42 - protein, 5 aa, Mus musculus
AYWMS

SEQ ID NO: 43 - protein, 17 aa, Mus musculus
EINPDSSTINCTPSLKD

SEQ ID NO: 44 - protein, 11 aa, Mus musculus
RLRPFWYFDVW

SEQ ID NO: 45 - protein, 16 aa, Mus musculus
RSSQSIVQSNGNTYLE

SEQ ID NO: 46 - protein, 7 aa, Mus musculus
KVSNRFS

SEQ ID NO: 47 - protein, 9 aa, Mus musculus
FQGSHVPRT

SEQ ID NO: 48 - protein, 6 aa, Mus musculus
TDYAWN

SEQ ID NO: 49 - protein, 16 aa, Mus musculus
YIFYSGSTTYTPSLKS

SEQ ID NO: 50 - protein, 8 aa, Mus musculus
GGYDVNYF

SEQ ID NO: 51 - protein, 11 aa, Mus musculus
LASQTIGAWLA

SEQ ID NO: 52 - protein, 7 aa, Mus musculus
AATRLAD

SEQ ID NO: 53 - protein, 9 aa, Mus musculus
QQFFSTPWT

SEQ ID NO: 54 - protein, 5 aa, Mus musculus
GYTMN

SEQ ID NO: 55 - protein, 16 aa, Mus musculus
LINPYNGVTSYNQKFK

SEQ ID NO: 56 - protein, 8 aa, Mus musculus
GDGNYWYF

SEQ ID NO: 57 - protein, 11 aa, Mus musculus
SASSSVTYMHW

SEQ ID NO: 58 - protein, 7 aa, Mus musculus
EISKLAS

SEQ ID NO: 59 - protein, 9 aa, Mus musculus
QEWNYPYTF

SEQ ID NO: 60 - protein, 117 aa, Mus musculus
QVQLQESGPGLVAPSQSLSITCTVSGFSLSRYNVHWVRQPPGKGLEWLGMIWDGGSTDYNSALKSRLSITKDNSKSQVFLKMNSLQTDDTAMYYCARNRYESGMDYWGQGTTVTVSS

SEQ ID NO: 61 - protein, 114 aa, Mus musculus
DIVMTQTPSSLTVTAGEKVTMSCKSSQSLLNSGNRKNYLTWYQQKPGQSPKLLIYWASTRESGVPDRFTGSGSGTDFTLIISSVQAEDLAVYYCQNDYTYPFTFGSGTKLEIKR

SEQ ID NO: 62 - protein, 119 aa, Mus musculus
EVKLLESGGGLVQPGGSLKLSCAASGFAFSAYWMSWVRQAPGKGLEWIGEINPDSSTINCTPSLKDKFIISRDNAKNTLSLQMNKVRSEDTALYYCARRLRPFWYFDVWGAGTTVTVSS

SEQ ID NO: 63 - protein, 112 aa, Mus musculus
DVLMTQTPLSLPVSLGDQASISCRSSQSIVQSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 64 - protein, 119 aa, Mus musculus
DVQLQESGPGLVKPSQSLSLTCTVTGYSITTDYAWNWIRQFPGNKLEWMGYIFYSGSTTYTPSLKSRISITRDTSKNQFFLQLNSVTTEDTATYYCARGGYDVNYFDYWGQGTTLTVSS

SEQ ID NO: 65 - protein, 107 aa, Mus musculus
DIQMTQSPASQSASLGESVTITCLASQTIGAWLAWYQQKPGKSPQLLIYAATRLADGVPSRFSGSGSGTKFSFKISSLQAEDFVSYYCQQFFSTPWTFGGGTKLEIK

SEQ ID NO: 66 - protein, 119 aa, Mus musculus
EVQLQQSGPELLKPGASMKISCKASGYSFTGYTMNWVKQSHGKNLEWIGLINPYNGVTSYNQKFKGKATLTVAKSSSTAYMELLSLTSEDSAVYYCARGDGNYWYFDVWGAGTTVTVSS

SEQ ID NO: 67 - protein, 105 aa, Mus musculus
EIVLTQSPAITAASLGQKVTITCSASSSVTYMHWYQQKSGTSPKPWIYEISKLASGVPVRFSGSGSGTSYSLTISSMEAEDAAIYYCQEWNYPYTFGGGTKLEIK

SEQ ID NO: 68 - protein, 119 aa, Mus musculus
EVQLQQSGPELVKPGASMKISCKASGYSFTGYTMNWVKQSHGKNLEWIGLINPYSGITSYNQNFKGKATLTVDKSSSTAYMELLNLTSEDSAVYYCARGDGNYWYFDVWGAGTTVTVSS

SEQ ID NO: 69 - protein, 105 aa, Mus musculus
EIILTQSPAITAASLGQKVTITCSASSSVSYMHWYQQKSGTSPKPWIYEISKLASGVPARFSGSGSGTSYSLTISSMEAEDAAIYYCQYWNYPYTFGGGTKLEIK

SEQ ID NO: 70 - protein, 5 aa, Mus musculus
GYTMN

SEQ ID NO: 71 - protein, 16 aa, Mus musculus
LINPYSGITSYNQNFK

SEQ ID NO: 72 - protein, 8 aa, Mus musculus
GDGNYWYF

SEQ ID NO: 73 - protein, 11 aa, Mus musculus
SASSSVSYMHW

SEQ ID NO: 74 - protein, 7 aa, Mus musculus
EISKLAS

SEQ ID NO: 75 - protein, 9 aa, Mus musculus
QYWNYPYTF

SEQ ID NO: 76 - protein, 12 aa, Cricetulus migratorius
GFSITTSGYYWT

SEQ ID NO: 77 - protein, 16 aa, Cricetulus migratorius
YIGYNSKTYYNPSLKS

SEQ ID NO: 78 - protein, 12 aa, Cricetulus migratorius
SLYGGYKDAFDS

SEQ ID NO: 79 - protein, 11 aa, Cricetulus migratorius
KASQSIGTSLH

SEQ ID NO: 80 - protein, 7 aa, Cricetulus migratorius
FASRSIT

SEQ ID NO: 81 - protein, 9 aa, Cricetulus migratorius
QQSPGFPPT

SEQ ID NO: 82 - protein, 122 aa, Cricetulus migratorius
QIQLQESGPGLVKPSQSLSLTCSVTGFSITTSGYYWTWIRQFPGKKLEWMGYIGYNSKTYYNPSLKSRISITRDTSKNQFLLHLNSVTTEDTATYYCARSLYGGYKDAFDSWGQGTLVTVSS

SEQ ID NO: 83 - protein, 108 aa, Cricetulus migratorius
DVVLTQTPATLSAIPGERVTMTCKASQSIGTSLHWYQHRPNETPRLLIKFASRSITGIPSRFSGSGSGTDFTLGINNLEAEDFAIYYCQQSPGFPPTFGSGTKLEINR

SEQ ID NO: 84 - protein, 12 aa, synthetic construct
  REGION 1..12: note = hCDR1 TSLPR-012_166
GFSITTSGYYWS

SEQ ID NO: 85 - protein, 16 aa, synthetic construct
  REGION 1..16: note = hCDR2 TSLPR-012_141
YIGYNSKTYYNPALKS

SEQ ID NO: 86 - protein, 11 aa, synthetic construct
  REGION 1..11: note = lCDR1 TSLPR-012_141
RASQSIGTSLH

SEQ ID NO: 87 - protein, 7 aa, synthetic construct
  REGION 1..7: note = lCDR2 TSLPR-012_141
FASRLQS

SEQ ID NO: 88 - protein, 7 aa, synthetic construct
  REGION 1..7: note = lCDR2 TSLPR-012_75
FASRSIS

SEQ ID NO: 89 - protein, 122 aa, synthetic construct
  REGION 1..122: note = sequence TSLPR-012_141 (humanized) HC variable region
QVQLVESGGGLVKPGGSLRLSCAASGFSITTSGYYWTWIRQFPGKGLEWMGYIGYNSKTYYNPALKSRITISRDNAKNSLYLQMNSLRAEDTAVYYCARSLYGGYKDAFDSWGQGTLVTVSS

SEQ ID NO: 90 - protein, 108 aa, synthetic construct
  REGION 1..108: note = sequence TSLPR-012_141 (humanized) LC variable region
DIQMTQSPSSLSASVGDRVTITCRASQSIGTSLHWYQQKPGKAPKLLIKFASRLQSGVPSRFSGSGSGTDFTLTISSLQPEDFATYYCQQSPGFPPTFGQGTKLEIKR

SEQ ID NO: 91 - protein, 108 aa, synthetic construct
  REGION 1..108: note = sequence TSLPR-012_75 (humanized) LC variable region
EIVLTQSPDFQSVTPKEKVTITCRASQSIGTSLHWYQQKPDQSPKLLIKFASRSISGVPSRFSGSGSGTDFTLTINSLEAEDAATYYCQQSPGFPPTFGQGTKLEIKR

SEQ ID NO: 92 - protein, 122 aa, synthetic construct
  REGION 1..122: note = sequence TSLPR-012_166 (humanized) HC variable region
QVQLQESGPGLVKPSQTLSLTCTVSGFSITTSGYYWSWIRQHPGKGLEWIGYIGYNSKTYYSPSLKSRVTISRDTSKNQFSLKLSSVTAADTAVYYCARSLYGGYKDAFDSWGQGTLVTVSS

SEQ ID NO: 93 - protein, 108 aa, synthetic construct
  REGION 1..108: note = sequence TSLPR-012_166 (humanized) LC variable region
DIQMTQSPSSLSASVGDRVTITCRASQSIGTSLHWYQHRPGETPKLLIKFASRSISGVPSRFSGSGSGTDFTLTISSLQPEDFATYYCQQSPGFPPTFGQGTKLEIKR

SEQ ID NO: 94 - protein, 108 aa, synthetic construct
  REGION 1..108: note = sequence TSLPR-012_189 (humanized) LC variable region
EIVMTQSPATLSVSPGERATLSCRASQSIGTSLHWYQQKPGQAPRLLIKFASRSITGIPARFSGSGSGTEFTLTISSLQSEDFAVYYCQQSPGFPPTFGQGTKLEIKR

SEQ ID NO: 95 - protein, 10 aa, synthetic construct
  REGION 1..10: note = Synthetic peptide
GYIFTDYAMH

SEQ ID NO: 96 - protein, 17 aa, synthetic construct
  REGION 1..17: note = Synthetic peptide
TFIPLLDTSDYAQKFQG

SEQ ID NO: 97 - protein, 11 aa, synthetic construct
  REGION 1..11: note = Synthetic peptide
MGVTHSYVMDA

SEQ ID NO: 98 - protein, 11 aa, synthetic construct
  REGION 1..11: note = Synthetic peptide
RASQPISISVH

SEQ ID NO: 99 - protein, 7 aa, synthetic construct
  REGION 1..7: note = Synthetic peptide
FASQSIS

SEQ ID NO: 100 - protein, 9 aa, synthetic construct
  REGION 1..9: note = Synthetic peptide
QQTFSLPYT

SEQ ID NO: 101 - protein, 120 aa, synthetic construct
  REGION 1..120: note = Synthetic peptide
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSS

SEQ ID NO: 102 - protein, 109 aa, synthetic construct
  REGION 1..109: note = Synthetic peptide
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRT

SEQ ID NO: 103 - protein, 450 aa, synthetic construct
  REGION 1..450: note = Synthetic peptide
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 214 aa, synthetic construct
  REGION 1..214: note = Synthetic peptide
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 448 aa, synthetic construct
  REGION 1..448: note = Synthetic Polypeptide
  REGION 1..448: note = MISC_FEATURE - Heavy chain
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 106 - protein, 214 aa, synthetic construct
  REGION 1..214: note = Synthetic Polypeptide
  REGION 1..214: note = MISC_FEATURE - Light Chain
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS